SEQUENCE LISTING (18 sequences)

SEQ ID NO: 1 - protein, 16 aa, Drosophila antennapedia
RQIKIWFQNRRMKWKK

SEQ ID NO: 2 - protein, 14 aa, Human immunodeficiency virus 1
GRKKRRQRRRPPQC

SEQ ID NO: 3 - protein, 27 aa, Artificial Sequence
  Description of Artificial Sequence Synthetic peptide
GALFLGWLGAAGSTMGAWSQPKKKRKV

SEQ ID NO: 4 - protein, 18 aa, Unknown
  Description of Unknown Organism Unknown Murinae PVEC peptide
LLIILRRRIRKQAHAHSK

SEQ ID NO: 5 - protein, 26 aa, Unknown
  Description of Unknown Organism Unknown Transportan peptide
GWTLNSAGYLLKINLKALAALAKKIL

SEQ ID NO: 6 - protein, 18 aa, Artificial Sequence
  Description of Artificial Sequence Synthetic peptide
KLALKLALKALKAALKLA

SEQ ID NO: 7 - protein, 9 aa, Artificial Sequence
  Description of Artificial Sequence Synthetic peptide
RRRRRRRRR

SEQ ID NO: 8 - protein, 10 aa, Artificial Sequence
  Description of Artificial Sequence Synthetic peptide
KFFKFFKFFK

SEQ ID NO: 9 - protein, 37 aa, Homo sapiens
LLGDFFRKSKEKIGKEFKRIVQRIKDFLRNLVPRTES

SEQ ID NO: 10 - protein, 31 aa, Ascaris suum
SWLSKTAKKLENSAKKRISEGIAIAIQGGPR

SEQ ID NO: 11 - protein, 30 aa, Homo sapiens
ACYCRIPACIAGERRYGTCIYQGRLWAFCC

SEQ ID NO: 12 - protein, 36 aa, Homo sapiens
DHYNCVSSGGQCLYSACPIFTKIQGTCYRGKAKCCK

SEQ ID NO: 13 - protein, 12 aa, Unknown
  Description of Unknown Organism Unknown Bactenecin peptide
RKCRIVVIRVCR

SEQ ID NO: 14 - protein, 42 aa, Sus scrofa
RRRPRPPYLPRPRPPPFFPPRLPPRIPPGFPPRFPPRFPGKR

SEQ ID NO: 15 - protein, 13 aa, Bos taurus
ILPWKWPWWPWRR

SEQ ID NO: 16 - protein, 16 aa, Unknown
  Description of Unknown Organism Unknown membrane translocation peptide
AAVALLPAVLLALLAP

SEQ ID NO: 17 - protein, 11 aa, Artificial Sequence
  Description of Artificial Sequence Synthetic peptide
AALLPVLLAAP

SEQ ID NO: 18 - protein, 13 aa, Human immunodeficiency virus 1
GRKKRRQRRRPPQ

(19) United States
(12) Patent Application Publication
(10) Pub. No.: US 2009/0163705 A1
(43) Pub. Date: Jun. 25, 2009

(54) CATIONIC LIPIDS

(75) Inventors: Muthiah Manoharan, Cambridge, MA (US); Kallanthottahil G. Rajeev, Cambridge, MA (US); Muthusamy Jayraman, Cambridge, MA (US); K. Narayanannair Jayaprakash, Cambridge, MA (US)

Correspondence Address: LOWRIE, LANDO & ANASTASI, LLP, ONE MAIN STREET, SUITE 1100, CAMBRIDGE, MA, 02142, US

(73) Assignee: ALNYLAM PHARMACEUTICALS, INC., Cambridge, MA (US)

(21) Appl. No.: 12/123,922

(22) Filed: May 20, 2008

Related U.S. Application Data
(60) Provisional application No. 60/939,204, filed on May 21, 2007.

(51) Int. Cl. C07J 43/00; C07D 207/16

(52) U.S. Cl. 540/110; 548/532

(57) ABSTRACT

Cyclic lipid moieties are described herein.

### CROSS-REFERENCE TO RELATED APPLICATIONS

This application claims priority to U.S. provisional application Ser. No. 60/939,204 filed May 21, 2007, which is incorporated herein by reference in its entirety.

### TECHNICAL FIELD

This invention relates to compositions and methods useful in administering nucleic acid based therapies, for example association complexes such as liposomes and lipoplexes.

### BACKGROUND

The opportunity to use nucleic acid based therapies holds significant promise, providing solutions to medical problems that could not be addressed with current, traditional medicines. The location and sequences of an increasing number of disease-related genes are being identified, and clinical testing of nucleic acid-based therapeutics for a variety of diseases is now underway.

One method of introducing nucleic acids into a cell is mechanically, using direct microinjection. However this method is not generally effective for systemic administration to a subject.

Systemic delivery of a nucleic acid therapeutic requires distributing nucleic acids to target cells and then transferring the nucleic acid across a target cell membrane intact and in a form that can function in a therapeutic manner.

Viral vectors have, in some instances, been used clinically successfully to administer nucleic acid based therapies. However, while viral-vectors have the inherent ability to transport nucleic acids across cell membranes, they can pose risks. One such risk involves the random integration of viral genetic sequences into patient chromosomes, potentially damaging the genome and possibly inducing a malignant transformation. Another risk is that the viral vector may revert to a pathogenic genotype either through mutation or genetic exchange with a wild type virus.

Lipid-based vectors have also been used in nucleic acid therapies and have been formulated in one of two ways. In one method, the nucleic acid is introduced into preformed liposomes or lipoplexes made of mixtures of cationic lipids and neutral lipids. The complexes thus formed have undefined and complicated structures and the transfection efficiency is severely reduced by the presence of serum. The second method involves the formation of DNA complexes with mono- or poly-cationic lipids without the presence of a neutral lipid. These complexes are prepared in the presence of ethanol and are not stable in water. Additionally, these complexes are adversely affected by serum (see, Behr, Acc. Chem. Res. 26:274-78 (1993)).

### SUMMARY

The invention features novel lipid moieties including a cyclic component, for example, that can be used to link to components together, for example two lipid components.

In one aspect, the invention features a compound of formula (I),

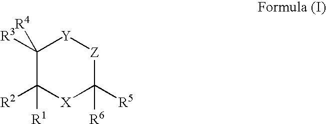

wherein:

X is NR⁷ or CH₂;

Y is NR⁸, O, S, CR⁹R¹⁰, or absent;

Z is CR¹¹R¹² or absent;

each of R¹, R², R³, R⁴, R⁵, R⁶, R⁹, R¹⁰, R¹¹, and R¹² is, independently, H, (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙO—N═CR¹⁶, (CH₂)N—N═CR¹⁶, a single D or L amino acid, a D or L di, tri, tetra or penta peptide, a combination of a D and L di, tri, tetra and penta peptide; or an oligopeptide; a PEG moiety, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle or heteroaryl (e.g. triazole);
each R⁷ and R⁸, for each occurrence, is independently H, C₁-C₃₀ alkyl, C₂-C₃₀ alkenyl, C₂-C₃₀ alkynyl, C(O)OR¹³, C(O)R¹⁶, SO₂R¹⁶, R^{d}, or a nitrogen protecting group such as BOC, Fmoc or benzyl;
R¹³ for each occurrence, is independently H, alkyl alkenyl, alkynyl, or R^{d}, each of which is optionally substituted with 1-3 nitrogen containing moieties selected from the group consisting of NR¹⁸R¹⁹ or a nitrogen containing heterocycle with one or more nitrogens;
each R¹⁴ and R¹⁵, for each occurrence, is independently H, alkyl alkenyl, or alkynyl, or R^{d}, each of which is optionally substituted with 1-3 nitrogen containing moieties selected from the group consisting of NR¹⁸R¹⁹ or a nitrogen containing heterocycle with one or more nitrogens;
R¹⁶, for each occurrence, is alkyl alkenyl, alkynyl, R^{d}, or —C₁₋₁₀alkylNR¹⁴C(O)R^{d}, each of which is optionally substituted with 1-3 nitrogen containing moieties selected from the group consisting of NR¹⁸R¹⁹ or a nitrogen containing heterocycle with one or more nitrogens;
R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};
each R^{L} and R^{L′} is independently H, alkyl alkenyl, alkynyl or R^{d}, each of which is optionally substituted with 1-3 nitrogen containing moieties selected from the group consisting of NR¹⁸R¹⁹ or a nitrogen containing heterocycle with one or more nitrogens; each R¹⁸ and R¹⁹, for each occurrence, is independently, H, alkyl alkenyl, alkynyl, or a nitrogen protecting group such as BOC, Fmoc or benzyl;
m is 0, 1, or 2
each n is independently 0 to 20.
In one embodiment, formula (I) contains at least one lipophilic group and at least one cationic group.

In some embodiments, each of R¹, R², R³, R⁴, R⁵, R⁶, R⁹, R¹⁰, R¹¹, and R¹² is, independently, H, (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R¹⁵; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙ, NR¹⁴C(O)R¹⁶, (CH₂)ₙO—N═CR¹⁶, (CH₂)N—N═CR¹⁶, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle, heteroaryl (e.g triazole).

In some embodiments, X is NR⁷. In some embodiments, R⁷ is H. In some embodiments, R⁷ is a nitrogen protecting group, for example BOC. In some embodiments, R⁷ is C(O)R¹⁶. In one embodiment R⁷ is SO₂R¹⁶.

In some embodiments, R¹⁶ is alkyl substituted with 1-3 NR¹⁸R¹⁹, for example, R¹⁶ is alkyl substituted with 2 NR¹⁸R¹⁹. In some embodiments, each NR¹⁸R¹⁹ is NH₂. In some embodiments, one NR¹⁸R¹⁹ is NH₂. In some embodiments, one NR¹⁸R¹⁹ is NMe₂. In some embodiments, R¹⁸ is H and R¹⁹ is Me of each NR¹⁸R¹⁹. In some embodiments, R¹⁸ is H and R¹⁹ is Me of one NR¹⁸R¹⁹ and R¹⁸ and R¹⁹ is H for the second NR¹⁸R¹⁹. In some embodiments, R¹⁸ is H and R¹⁹ is Me of one NR¹⁸R¹⁹ and R¹⁸ and R¹⁹ is Me for the second NR¹⁸R¹⁹. In some embodiments, R¹⁶ is alkyl substituted with NH₂ and NMe₂.

In some embodiments, R¹⁶ is substituted with a nitrogen containing heterocyclyl. In some embodiments, R¹⁶ is further substituted by NR¹⁸R¹⁹. In some embodiments, wherein NR¹⁸R¹⁹ is NH₂. In some embodiments, the nitrogen containing heterocyclyl has 2 ring nitrogens. In some embodiments, the nitrogen containing heterocyclcyl is a nitrogen containing heteroaryl. In some embodiments, the nitrogen containing heteroaryl has 2 ring nitrogens. In some embodiments, the heteroaryl is an imidazolyl.

In some embodiments, R¹⁶ is alkyl substituted with NH₂ and imidazolyl.

In some embodiments, R¹⁶ is

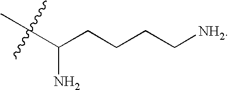

In some embodiments, R¹⁶ is

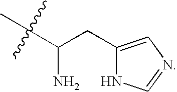

In some embodiments, Y is CR⁹R¹⁰. In some embodiments, R⁹ and R¹⁰ are both H.

In some embodiments, Z is absent.

In some embodiments, Y is CR⁹R¹⁰ and Z is absent. In some embodiments, R⁹ and R¹⁰ are both H. In some embodiments, R¹, R², R⁴, R⁶ are all H.

In some embodiments, Y is NR⁸.

In some embodiments, Z is CR¹¹R¹². In some embodiments, R¹¹ and R¹² are both H.

In some embodiments, Y is NR⁸ and Z is CR¹¹R¹².

In some embodiments, R¹ and R² are both H.

In some embodiments, R³ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R¹⁵; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙO—N═CR¹⁶, (CH₂)N—N═CR¹⁶, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle, heteroaryl (e.g triazole), where n is 0 or 1.

In some embodiments, R⁴ is H.

In some embodiments, R³ is OR¹³, NR¹⁴R¹⁵, C(O)NR¹⁴R¹⁵, or NR¹⁴C(O)R¹⁶.

In some embodiments, R³ is OR¹³, NR¹⁴R¹⁵, C(O)NR¹⁴R¹⁵, or NR¹⁴C(O)R¹⁶; and wherein R⁴ is H.

In some embodiments, R³ is NR¹⁴R¹⁵ or NR¹⁴C(O)R¹⁶.

In some embodiments, R³ is NR¹⁴R¹⁵ or NR¹⁴C(O)R¹⁶ and R⁴ is H.

In some embodiments, R³ is NR¹⁴C(O)R¹⁶.

In some embodiments, R¹⁶ is alkyl, for example, R¹⁶ is C₁₀₋₃₀ alkyl, R¹⁶ is C₁₀₋₁₈ alkyl, or R¹⁶ is C₁₅ is alkyl.

In some embodiments, R¹⁶ is alkenyl. In some embodiments, R¹⁶ is C₆-C₃₀ alkenyl. In some embodiments, R¹⁶ has a single double bond. In some embodiments, the double bond has a Z configuration. In some embodiments, R¹⁶ has two double bonds. In some embodiments, at least one of the double bonds has a Z configuration. In some embodiments, both of the double bonds have a Z configuration. In some embodiments, R¹⁶ has the following formula:

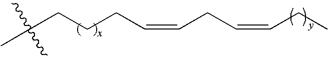

wherein

x is an integer from 1 to 8; and

y is an integer from 1-10. In some embodiments, R¹⁶ is

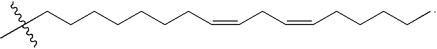

In some embodiments, at least one of the double bonds has an E configuration. In some embodiments, both of the double bonds have an E configuration. In some embodiments, R¹⁶ has the following formula:

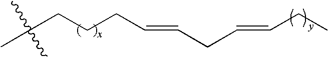

wherein
x is an integer from 1 to 8; and
y is an integer from 1-10. In some embodiments, R¹⁶ has three double bond moieties. In some embodiments, at least one of the double bonds has a Z configuration. In some embodiments, at least two of the double bonds have a Z configuration. In some embodiments, all three of the double bonds have a Z configuration. In some embodiments, R¹⁶ has the following formula:

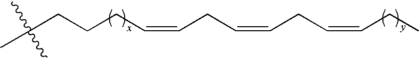

wherein
x is an integer from 1 to 8; and
y is an integer from 1-10. In some embodiments, at least one of the double bonds has an E configuration. In some embodiments, at least two of the double bonds have an E configuration. In some embodiments, all three of the double bonds have an E configuration. In some embodiments, R¹⁶ has the following formula:

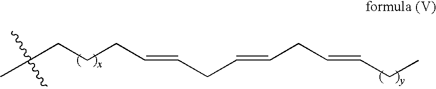

wherein

x is an integer from 1 to 8; and

y is an integer from 1-10.

In some embodiments, R¹⁶ is alkynyl.

In some embodiments, R¹⁶ is R^{d} or C₁-C₁₀ alkyl substituted with NHC(O)R^{d}. In some embodiments, R¹⁶ is R^{d}. In some embodiments, R¹⁶ is R^{d} and R^{d} is an unsubstituted cholesterol moiety. In some embodiments, R¹⁶ is C₁-C₁₀ alkyl substituted with NHC(O)R^{d}. In some embodiments, R^{d} is an unsubstituted cholesterol moiety. In some embodiments, R¹⁶ is (CH₂)₅NHC(O)R^{d}, and R^{d} is an unsubstituted cholesterol moiety. In some embodiments, R¹⁶ is a cholesterol moiety, substituted with C(O)OR^{L}, C(O)NR^{L}R^{L′}, R^{L}, S(O)ₘR^{L}, or S(O)ₘNR^{L}R^{L′}. In some embodiments, R¹⁶ is a cholesterol moiety, substituted with C(O)NR^{L}R^{L′}. In some embodiments, R^{L} is alkenyl and R^{L′} is H. In some embodiments, R^{L} has a Z configuration. In some embodiments, R^{L} is C¹⁸, alkenyl.

In some embodiments, R³ is NR¹⁴C(O)R¹⁶ and wherein R⁴ is H.

In some embodiments, R⁵ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙ NR¹⁴C(O)R¹⁶, (CH₂)ₙO—N═CR¹⁶; (CH₂)N—N═CR¹⁶, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle, heteroaryl (e.g. triazole); where n is 0 or 1. In some embodiments, R⁶ is H.

In some embodiments, R⁵ is C(O)OR¹³ or C(O)NR¹⁴R¹⁵. In some embodiments, R⁶ is H.

In some embodiments, R⁵ is C(O)NR¹⁴R¹⁵.

In some embodiments, R⁵ is C(O)NR¹⁴R¹⁵ and R⁶ is H.

In some embodiments, R¹⁴ is H.

In some embodiments, R¹⁵ is alkyl optionally substituted with 1-3 nitrogen containing moieties selected from the group consisting of NR¹⁸R¹⁹ or a nitrogen containing heterocyclyl.

In some embodiments, R¹⁵ is alkyl optionally substituted with 1-3 nitrogen containing moieties selected from the group consisting of NR¹⁸R¹⁹ or a nitrogen containing heterocyclyl and R¹⁴ is H.

In some embodiments, R¹⁵ is C₁-C₆ alkyl substituted with a nitrogen containing moiety selected from the group consisting of NR¹⁸R¹⁹ or a nitrogen containing heterocyclyl.

In some embodiments, R¹⁵ is C₁-C₆ alkyl substituted with a nitrogen containing moiety selected from the group consisting of NR¹⁸R¹⁹ or a nitrogen containing heterocyclyl and R¹⁴ is H.

In some embodiments, R¹⁵ is C₁-C₆ alkyl substituted NR¹⁸R¹⁹. In some embodiments, R¹⁸ and R¹⁹ are both alkyl. In some embodiments, R¹⁸ and R¹⁹ are both C₁-C₆ alkyl. In some embodiments, R¹⁸ and R¹⁹ are both methyl.

In some embodiments, wherein R¹⁵ is

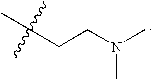

In some embodiments, R¹⁴ is H.

In some embodiments, R¹⁵ is C₁-C₆ alkyl substituted with a nitrogen containing heterocyclyl. In some embodiments, the nitrogen containing heterocyclyl has 2 ring nitrogens. In some embodiments, the nitrogen containing heteroaryl has 2 ring nitrogens. In some embodiments, heteroaryl is an imidazolyl. In some embodiments, R¹⁵ is

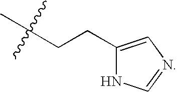

In some embodiments, both R¹⁴ and R¹⁵ are C₁-C₆ alkyl substituted NR¹⁸R¹⁹. In some embodiments, both R¹⁴ and R¹⁵ are

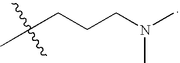

In some embodiments, one or both of R¹⁴ and R¹⁵ are alkyl. In some embodiments, one or both of R¹⁴ and R¹⁵ is C₁₀₋₃₀ alkyl. In some embodiments, one or both of R¹⁴ and R¹⁵ is C₁₀₋₁₈ alkyl. In some embodiments, one or both of R¹⁴ and R¹⁵ is C₁₂ alkyl.

In some embodiments, one or both of R¹⁴ and R¹⁵ is alkenyl. In some embodiments, one or both of R¹⁴ and R¹⁵ is C₆-C₃₀ alkenyl. In some embodiments, one or both of R¹⁴ and R¹⁵ has a single double bond. In some embodiments, the double bond has a Z configuration. In some embodiments, one or both of R¹⁴ and R¹⁵ has two double bonds. In some embodiments, at least one of the double bonds have a Z configuration. In some embodiments, both of the double bonds have a Z configuration. In some embodiments, one or both of R¹⁴ and R¹⁵ has the following formula:

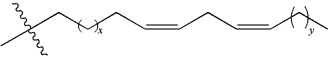

wherein

x is an integer from 1 to 8; and

y is an integer from 1-10. one or both of R¹⁴ and R¹⁵ is

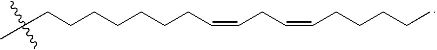

In some embodiments, at least one of the double bonds has an E configuration. In some embodiments, both of the double bonds have an E configuration. In some embodiments, one or both of R¹⁴ and R¹⁵ has the following formula:

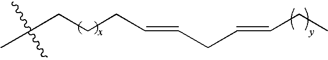

wherein

x is an integer from 1 to 8; and

y is an integer from 1-10. In some embodiments, one or both of R¹⁴ and R¹⁵ has three double bond moieties. In some embodiments, at least one of the double bonds has a Z configuration. In some embodiments, at least two of the double bonds have a Z configuration. In some embodiments, all three of the double bonds have a Z configuration. In some embodiments, one or both of R¹⁴ and R¹⁵ has the following formula:

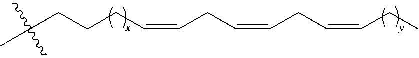

wherein

x is an integer from 1 to 8; and

y is an integer from 1-10. In some embodiments, at least one of the double bonds have an E configuration. In some embodiments, at least two of the double bonds have an E configuration. In some embodiments, all three of the double bonds have an E configuration. In some embodiments, one or both of R¹⁴ and R¹⁵ has the following formula:

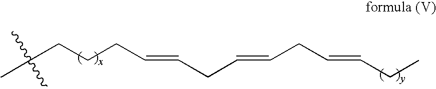

wherein

x is an integer from 1 to 8; and

y is an integer from 1-10.

In some embodiments, one or both of R¹⁴ and R¹⁵ is alkynyl.

In some embodiments, R¹, R², R⁴, R⁶ are all H.

In some embodiments, R¹, R², R⁴, R⁶ are all H and Z is absent.

In one aspect, the invention features a compound of formula (II)

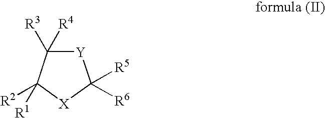

X is NR⁷ or CH₂;

Y is NR⁸, O, S, CR⁹R¹⁰, or absent;

each of R¹, R², R³, R⁴, R⁵, R⁶, R⁹, and R¹⁰ is, independently, H, (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙO—N═CR¹⁶, (CH₂)N—N═CR¹⁶, a single D or L amino acid, a D or L di, tri, tetra or penta peptide, a combination of a D and L di, tri, tetra and penta peptide, an oligopeptide; a PEG moiety, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle or heteroaryl (e.g. triazole);

each R⁷ and R⁸, for each occurrence, is independently H, C₁-C₃₀ alkyl, C₂-C₃₀ alkenyl, C₂-C₃₀ alkynyl, C(O)OR¹³, C(O)R¹⁶, SO₂R¹⁶, R^{d}, or a nitrogen protecting group such as BOC, Fmoc or benzyl;

R¹³, for each occurrence, is independently H, alkyl alkenyl, alkynyl, or R^{d}, each of which is optionally substituted with 1-3 nitrogen containing moieties selected from the group consisting of NR¹⁸R¹⁹ or a nitrogen containing heterocyclyl;

each R¹⁴ and R¹⁵, for each occurrence, is independently H, alkyl alkenyl, or alkynyl, or R^{d}, each of which is optionally substituted with 1-3 nitrogen containing moieties selected from the group consisting of NR¹⁸R¹⁹ or a nitrogen containing heterocyclyl;

R¹⁶, for each occurrence, is alkyl alkenyl, alkynyl, R^{d}, each of which is optionally substituted with 1-3 nitrogen containing moieties selected from the group consisting of NR¹⁸R¹⁹ or a nitrogen containing heterocyclyl;

R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};

each R^{L} and R^{L′} is independently H, alkyl alkenyl, alkynyl or R^{d}, each of which is optionally substituted with 1-3 nitrogen containing moieties selected from the group consisting of NR¹⁸R¹⁹ or a nitrogen containing heterocyclyl;

each R¹⁸ and R¹⁹, for each occurrence, is independently, H, alkyl alkenyl, alkynyl, or a nitrogen protecting group such as BOC, Fmoc or benzyl;

m is 0, 1, or 2

each n is independently 0, 1, 2, 3, or 4.

In one embodiment, formula (II) contains at least one lipophilic group and one cationic group.

In some embodiments, each of R¹, R², R³, R⁴, R⁵, R⁶, R⁹, and R¹⁰ is, independently, H, (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙO—N═CR¹⁶, (CH₂)N—N═CR¹⁶, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle or heteroaryl (e.g. triazole).

In some embodiments,

X is NR⁷ or CH₂;

Y is NR⁸, O, S, CR⁹R¹⁰;

each of R¹, R², R³, R⁴, R⁵, R⁶, R⁹, and R¹⁰ is, independently, H, (CH₂)ₙOR¹³, (CH₂)ₙC(O)OR¹³, (CH₂)ₙOC(O)R¹⁶, (CH₂)ₙS(O)ₘR¹³ (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙ NR¹⁴C(O)R¹⁶, (CH₂)ₙO—N═CR¹⁶; (CH₂)N—N═CR¹⁶, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀alkenyl, C₂-C₃₀ alkynyl, or

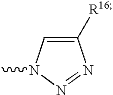

each R⁷ and R⁸ is independently H, C₁-C₆ alkyl, SO₂R¹⁶ or a nitrogen protecting group, e.g., a C(O)Oalkyl moiety such as BOC, or C(O)R¹⁶;

R¹³, for each occurrence, is independently H, alkyl alkenyl, or alkynyl;

each R¹⁴ and R¹⁵, for each occurrence, is independently H, alkyl alkenyl, or alkynyl, each of which is optionally substituted with 1-3 nitrogen containing moieties selected from the group consisting of NR¹⁸R¹⁹ or a nitrogen containing heterocyclyl;

R¹⁶, for each occurrence, is alkyl alkenyl, alkynyl, R^{d} or C₁-C₁₀ alkyl substituted with NHC(O)R^{d};

R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};

each R^{L} and R^{L′} is independently H, alkyl, alkenyl, or alkynyl;

m is 0, 1, or 2

n is an integer from 1 to 20.

In some embodiments, Y is CR⁹R¹⁰. In some embodiments, R⁹ and R¹⁰ are H.

In some embodiments, R¹ and R² are H.

In some embodiments, X is NR⁷. In some embodiments, R⁷ is H.

In some embodiments, X is NR⁷. In some embodiments, R⁷ is H. In some embodiments, R⁷ is a nitrogen protecting group, for example BOC.

In some embodiments, R⁷ is C(O)R¹⁶.

In some embodiments, R⁷ is SO₂R¹⁶.

In some embodiments, R¹⁶ is alkyl substituted with 1-3 NR¹⁸R¹⁹, for example, R¹⁶ is alkyl substituted with 2 NR¹⁸R¹⁹. In some embodiments, each NR¹⁸R¹⁹ is NH₂. In some embodiments, one NR¹⁸R¹⁹ is NH₂. In some embodiments, one NR¹⁸R¹⁹ is NMe₂. In some embodiments, R¹⁸ is H and R¹⁹ is Me of each NR¹⁸R¹⁹. In some embodiments, R¹⁸ is H and R¹⁹ is Me of one NR¹⁸R¹⁹ and R¹⁸ and R¹⁹ is H for the second NR¹⁸R¹⁹. In some embodiments, R¹⁸ is H and R¹⁹ is Me of one NR¹⁸R¹⁹ and R¹⁸ and R¹⁹ is Me for the second NR¹⁸R¹⁹. In some embodiments, R¹⁶ is alkyl substituted with NH₂ and NMe₂.

In some embodiments, R¹⁶ is substituted with a nitrogen containing heterocyclyl. In some embodiments, R⁶ is further substituted by NR¹⁸R¹⁹. In some embodiments, wherein NR¹⁸R¹⁹ is NH₂. In some embodiments, the nitrogen containing heterocyclyl has 2 ring nitrogens. In some embodiments, the nitrogen containing heterocyclcyl is a nitrogen containing heteroaryl. In some embodiments, the nitrogen containing heteroaryl has 2 ring nitrogens. In some embodiments, the heteroaryl is an imidazolyl.

In some embodiments, R¹⁶ is alkyl substituted with NH₂ and imidazolyl. In some embodiments, R¹⁶ is

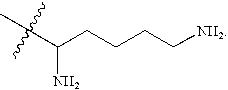

In some embodiments, R¹⁶ is

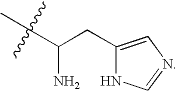

In some embodiments, Y is CR⁹R¹⁰. In some embodiments, R⁹ and R¹⁰ are both H.

In some embodiments, R⁹ and R¹⁰ are both H. In some embodiments, R¹, R², R⁴, R⁶ are all H.

In some embodiments, Y is NR⁸. In some embodiments, R¹ and R² are both H.

In some embodiments, R³ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙ NR¹⁴C(O)R¹⁶, (CH₂)ₙ O—N═CR¹⁶; (CH₂)N—N═CR¹⁶;

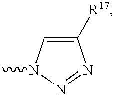

where n is 0 or 1. In some embodiments, R⁴ is H.

In some embodiments, R³ is OR¹³, NR¹⁴R¹⁵, C(O)NR¹⁴R¹⁵, or NR¹⁴C(O)R¹⁶.

In some embodiments, R³ is OR¹³, NR¹⁴R¹⁵, C(O)NR¹⁴R¹⁵, or NR¹⁴C(O)R¹⁶; and wherein R⁴ is H.

In some embodiments, R³ is NR¹⁴R¹⁵ or NR¹⁴C(O)R¹⁶.

In some embodiments, R³ is NR¹⁴R¹⁵ or NR¹⁴C(O)R¹⁶ and R⁴ is H.

In some embodiments, R³ is NR¹⁴C(O)R¹⁶.

In some embodiments, R¹⁶ is alkyl, for example, R¹⁶ is Cl₁₀₋₃₀ alkyl, R¹⁶ is C₁₀₋₁₈ alkyl, or R¹⁶ is C₁₅ alkyl.

In some embodiments, R¹⁶ is alkenyl. In some embodiments, R¹⁶ is C₆-C₃₀ alkenyl. In some embodiments, R¹⁶ has a single double bond. In some embodiments, the double bond has a Z configuration: In some embodiments, R¹⁶ has two double bonds. In some embodiments, at least one of the double bonds has a Z configuration. In some embodiments, both of the double bonds have a Z configuration. In some embodiments, R¹⁶ has the following formula:

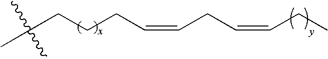

wherein

x is an integer from 1 to 8; and

y is an integer from 1-10. In some embodiments, R¹⁶ is

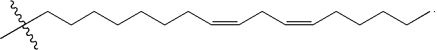

In some embodiments, at least one of the double bonds has an E configuration. In some embodiments, both of the double bonds have an E configuration. In some embodiments, R¹⁶ has the following formula:

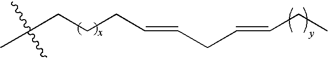

wherein

x is an integer from 1 to 8; and

y is an integer from 1-10. In some embodiments, R¹⁶ has three double bond moieties. In some embodiments, at least one of the double bonds has a Z configuration. In some embodiments, at least two of the double bonds have a Z configuration. In some embodiments, all three of the double bonds have a Z configuration. In some embodiments, R¹⁶ has the following formula:

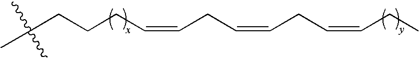

wherein

x is an integer from 1 to 8; and

y is an integer from 1-10. In some embodiments, at least one of the double bonds has an E configuration. In some embodiments, at least two of the double bonds have an E configuration. In some embodiments, all three of the double bonds have an E configuration. In some embodiments, R¹⁶ has the following formula:

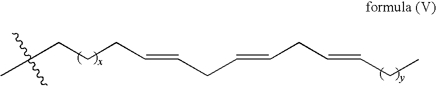

wherein

x is an integer from 1 to 8; and

y is an integer from 1-10.

In some embodiments, R¹⁶ is alkynyl.

In some embodiments, R¹⁶ is R^{d} or C₁-C₁₀ alkyl substituted with NHC(O)R^{d}. In some embodiments, R¹⁶ is R^{d}. In some embodiments, R¹⁶ is R^{d} and R^{d} is an unsubstituted cholesterol moiety. In some embodiments, R¹⁶ is C₁-C₁₀ alkyl substituted with NHC(O)R^{d}. In some embodiments, R^{d} is an unsubstituted cholesterol moiety. In some embodiments, R¹⁶ is (CH₂)₅NHC(O)R^{d}, and R^{d} is an unsubstituted cholesterol moiety. In some embodiments, R¹⁶ is a cholesterol moiety, substituted with C(O)OR^{L}, C(O)NR^{L}R^{L′}, R^{L}, S(O)ₘR^{L}, or S(O)ₘNR^{L}R^{L′}. In some embodiments, R¹⁶ is a cholesterol moiety, substituted with C(O)NR^{L}R^{L′}. In some embodiments, R^{L} is alkenyl and R^{L′} is H. In some embodiments, R^{L} has a Z configuration. In some embodiments, R^{L} is C¹⁸ alkenyl.

In some embodiments, R³ is NR¹⁴C(O)R¹⁶ and wherein R⁴ is H.

In some embodiments, R⁵ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙ O—N═CR¹⁶; (CH₂)N—N═CR¹⁶; or

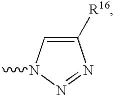

where n is 0 or 1. In some embodiments, R⁶ is H.

In some embodiments, R⁵ is C(O)OR¹³ or C(O)NR¹⁴R¹⁵. In some embodiments, R⁶ is H.

In some embodiments, R⁵ is C(O)NR¹⁴R¹⁵.

In some embodiments, R⁵ is C(O)NR¹⁴R¹⁵ and R⁶ is H.

In some embodiments, R¹⁴ is H.

In some embodiments, R¹⁵ is alkyl optionally substituted with 1-3 nitrogen containing moieties selected from the group consisting of NR¹⁸R¹⁹ or a nitrogen containing heterocyclyl.

In some embodiments, R¹⁵ is alkyl optionally substituted with 1-3 nitrogen containing moieties selected from the group consisting of NR¹⁸R¹⁹ or a nitrogen containing heterocyclyl and R¹⁴ is H.

In some embodiments, R¹⁵ is C₁-C₆ alkyl substituted with a nitrogen containing moiety selected from the group consisting of NR¹⁸R¹⁹ or a nitrogen containing heterocyclyl.

In some embodiments, R¹⁵ is C₁-C₆ alkyl substituted with a nitrogen containing moiety selected from the group consisting of NR¹⁸R¹⁹ or a nitrogen containing heterocyclyl and R¹⁴ is H.

In some embodiments, R¹⁵ is C₁-C₆ alkyl substituted NR¹⁸R¹⁹. In some embodiments, R¹⁸ and R¹⁹ are both alkyl. In some embodiments, R¹⁸ and R¹⁹ are both C₁-C₆ alkyl. In some embodiments, R¹⁸ and R¹⁹ are both methyl.

In some embodiments, wherein R⁵ is

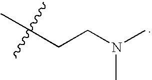

In some embodiments, R¹⁴ is H.

In some embodiments, R¹⁵ is C₁-C₆ alkyl substituted with a nitrogen containing heterocyclyl. In some embodiments, the nitrogen containing heterocyclyl has 2 ring nitrogens. In some embodiments, the nitrogen containing heteroaryl has 2 ring nitrogens. In some embodiments, heteroaryl is an imidazolyl. In some embodiments, R¹⁵ is

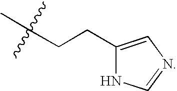

In some embodiments, both R¹⁴ and R¹⁵ are C₁-C₆ alkyl substituted NR¹⁸R¹⁹. In some embodiments, both R¹⁴ and R¹⁵ are

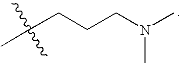

In some embodiments, one or both of R¹⁴ and R¹⁵ are alkyl. In some embodiments, one or both of R¹⁴ and R¹⁵ is C₁₀₋₃₀ alkyl. In some embodiments, one or both of R¹⁴ and R¹⁵ is Cl₁₀₋₁₈ alkyl. In some embodiments, one or both of R¹⁴ and R¹⁵ is C₁₂ alkyl.

In some embodiments, one or both of R¹⁴ and R¹⁵ is alkenyl. In some embodiments, one or both of R¹⁴ and R¹⁵ is C₆-C₃₀ alkenyl. In some embodiments, one or both of R¹⁴ and R¹⁵ has a single double bond. In some embodiments, the double bond has a Z configuration. In some embodiments, one or both of R¹⁴ and R¹⁵ has two double bonds. In some embodiments, at least one of the double bonds have a Z configuration. In some embodiments, both of the double bonds have a Z configuration. In some embodiments, one or both of R¹⁴ and R¹⁵ has the following formula:

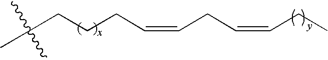

wherein

x is an integer from 1 to 8; and

y is an integer from 1-10. one or both of R¹⁴ and R¹⁵ is

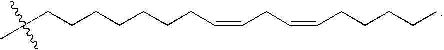

In some embodiments, at least one of the double bonds has an E configuration. In some embodiments, both of the double bonds have an E configuration. In some embodiments, one or both of R¹⁴ and R¹⁵ has the following formula:

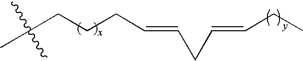

wherein

x is an integer from 1 to 8; and

y is an integer from 1-10. In some embodiments, one or both of R¹⁴ and R¹⁵ has three double bond moieties. In some embodiments, at least one of the double bonds has a Z configuration. In some embodiments, at least two of the double bonds have a Z configuration. In some embodiments, all three of the double bonds have a Z configuration. In some embodiments, one or both of R¹⁴ and R¹⁵ has the following formula:

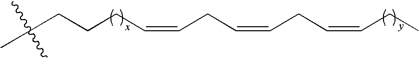

wherein

x is an integer from 1 to 8; and

y is an integer from 1-10. In some embodiments, at least one of the double bonds have an E configuration. In some embodiments, at least two of the double bonds have an E configuration. In some embodiments, all three of the double bonds have an E configuration. In some embodiments, one or both of R¹⁴ and R¹⁵ has the following formula:

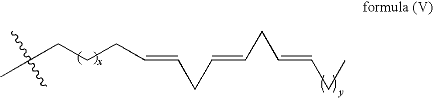

wherein

x is an integer from 1 to 8; and

y is an integer from 1-10.

In some embodiments, one or both of R¹⁴ and R¹⁵ is alkynyl.

In some embodiments, R¹, R², R⁴, R⁶ are all H.

In one aspect, the invention features a compound of formula (III)

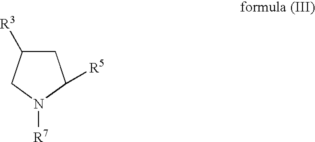

each of R³ and R⁵ is, independently, H, (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙ NR¹⁴C(O)R¹⁶, (CH₂)ₙO—N═CR¹⁶, a single D or L amino acid, a D or L di, tri, tetra or penta peptide, a combination of a D and L di, tri, tetra and penta peptide, an oligopeptide; a PEG moiety, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle or heteroaryl (e.g. triazole);

R⁷ is H, C₁-C₃₀ alkyl, C₂-C₃₀ alkenyl, C₂-C₃₀ alkynyl, C(O)OR¹³, C(O)R¹⁶, SO₂R¹⁶, R^{d}, or a nitrogen protecting group such as BOC, Fmoc or benzyl;

R¹³, for each occurrence, is independently H, alkyl alkenyl, alkynyl, or R^{d}, each of which is optionally substituted with 1-3 nitrogen containing moieties selected from the group consisting of NR¹⁸R¹⁹ or a nitrogen containing heterocyclyl;

each R¹⁴ and R¹⁵, for each occurrence, is independently H, alkyl alkenyl, or alkynyl, or R^{d}, each of which is optionally substituted with 1-3 nitrogen containing moieties selected from the group consisting of NR¹⁸R¹⁹ or a nitrogen containing heterocyclyl;

R¹⁶, for each occurrence, is alkyl alkenyl, alkynyl, R^{d}, each of which is optionally substituted with 1-3 nitrogen containing moieties selected from the group consisting of NR¹⁸R¹⁹ or a nitrogen containing heterocyclyl;

R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};

each R^{L} and R^{L′} is independently H, alkyl alkenyl, alkynyl or R^{d}, each of which is optionally substituted with 1-3 nitrogen containing moieties selected from the group consisting of NR¹⁸R¹⁹ or a nitrogen containing heterocyclyl;

each R¹⁸ and R¹⁹, for each occurrence, is independently, H, alkyl alkenyl, alkynyl, or a nitrogen protecting group such as BOC, Fmoc or benzyl;

m is 0, 1, or 2

each n is independently 0 to 20.

In one embodiment formula (III) contains at least one lipophilic group and at least one cationic group.

In some embodiments, each of R³ and R⁵ is, independently, H, (CH₂)ₙ—OR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙNR¹⁴R¹⁵, (CH₂)ₙC(O)NR¹⁴R¹⁵, (CH₂) OC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙ O—N═CR¹⁶, (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, or

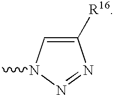

In some embodiments,

each of R³ and R⁵ is, independently, H, OR¹³, C(O)OR¹³, OC(O)R¹⁶, (CH₂)ₙOR¹³, (CH₂)ₙC(O)OR¹³, (CH₂)ₙOC(O)R¹⁶, S(O)ₘR¹³, or S(O)ₘNR¹⁴R^{15′}; NR¹⁴R¹⁵, (CH₂)ₙNR¹⁴R¹⁵, (CH₂)ₙC(O)NR¹⁴R¹⁵, C(O)NR¹⁴R¹⁵, NR¹⁴C(O)NR¹⁴R¹⁵, OC(O)NR¹⁴R¹⁵, NR¹⁴C(O)OR¹³, NR¹⁴C(O)R¹⁶, (CH₂)ₙNR¹⁴C(O)R¹⁶, O—N═CR¹⁶;

each R⁷ and R⁸ is independently H, C₁-C₆ alkyl, a nitrogen protecting group, e.g., a C(O)Oalkyl moiety such as BOC, or C(O)R¹⁶;

R¹³, for each occurrence, is independently H, alkyl alkenyl, or alkynyl;

each R¹⁴ and R¹⁵, for each occurrence, is independently H, alkyl alkenyl, or alkynyl, each of which is optionally substituted with 1-3 nitrogen containing moieties selected from the group consisting of NR¹⁸R¹⁹ or a nitrogen containing heterocyclyl;

R¹⁶, for each occurrence, is alkyl alkenyl, alkynyl, R^{d} or C₁-C₁₀ alkyl substituted with NHC(O)R^{d} or with 1-3 nitrogen containing moieties selected from the group consisting of NR¹⁸R¹⁹ or a nitrogen containing heterocyclyl;

R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};

each R^{L} and R^{L′} is independently H, alkyl alkenyl, or alkynyl;

m is 0, 1, or 2

n is an integer from 1 to 4.

In some embodiments, R⁷ is H.

In some embodiments, R⁷ is a nitrogen protecting group, for example BOC.

In some embodiments, R⁷ is C(O)R¹⁶.

In some embodiments, R¹⁶ is alkyl substituted with 1-3 NR¹⁸R¹⁹, for example, R¹⁶ is alkyl substituted with 2 NR¹⁸R¹⁹. In some embodiments, each NR¹⁸R¹⁹ is NH₂. In some embodiments, one NR¹⁸R¹⁹ is NH₂. In some embodiments, one NR¹⁸R¹⁹ is NMe₂. In some embodiments, R¹⁸ is H and R¹⁹ is Me of each NR¹⁸R¹⁹. In some embodiments, R¹⁸ is H and R¹⁹ is Me of one NR¹⁸R¹⁹ and R¹⁸ and R¹⁹ is H for the second NR¹⁸R¹⁹. In some embodiments, R¹⁸ is H and R¹⁹ is Me of one NR¹⁸R¹⁹ and R¹⁸ and R¹⁹ is Me for the second NR¹⁸R¹⁹. In some embodiments, R¹⁶ is alkyl substituted with NH₂ and NMe₂.

In some embodiments, R¹⁶ is substituted with a nitrogen containing heterocyclyl. In some embodiments, R¹⁶ is further substituted by NR¹⁸R¹⁹. In some embodiments, wherein NR¹⁸R¹⁹ is NH₂. In some embodiments, the nitrogen containing heterocyclyl has 2 ring nitrogens. In some embodiments, the nitrogen containing heterocyclcyl is a nitrogen containing heteroaryl. In some embodiments, the nitrogen containing heteroaryl has 2 ring nitrogens. In some embodiments, the heteroaryl is an imidazolyl.

In some embodiments, R¹⁶ is alkyl substituted with NH₂ and imidazolyl. In some embodiments, R¹⁶ is

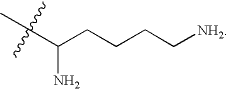

In some embodiments, R¹⁶ is

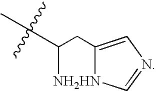

In some embodiments, R³ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙO—N═CR¹⁶; (CH₂)N—N═CR¹⁶;

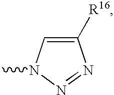

where n is 0 or 1. In some embodiments, R⁴ is H.

In some embodiments, R³ is OR¹³, NR¹⁴R¹⁵, C(O)NR¹⁴R¹⁵, or NR¹⁴C(O)R¹⁶.

In some embodiments, R³ is OR¹³, NR¹⁴R¹⁵, C(O)NR¹⁴R¹⁵, or NR¹⁴C(O)R¹⁶; and wherein R⁴ is H.

In some embodiments, R³ is NR¹⁴R¹⁵ or NR¹⁴C(O)R¹⁶.

In some embodiments, R³ is NR¹⁴R¹⁵ or NR¹⁴C(O)R¹⁶ and R⁴ is H.

In some embodiments, R³ is NR¹⁴C(O)R¹⁶.

In some embodiments, R¹⁶ is alkyl, for example, R¹⁶ is Cl₁₀₋₃₀ alkyl, R¹⁶ is C₁₀₋₁₈ alkyl, or R¹⁶ is C₁₅alkyl.

In some embodiments, R¹⁶ is alkenyl. In some embodiments, R¹⁶ is C₆-C₃₀ alkenyl. In some embodiments, R¹⁶ has a single double bond. In some embodiments, the double bond has a Z configuration. In some embodiments, R¹⁶ has two double bonds. In some embodiments, at least one of the double bonds has a Z configuration. In some embodiments, both of the double bonds have a Z configuration. In some embodiments, R¹⁶ has the following formula:

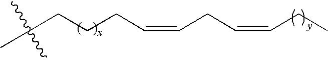

wherein

x is an integer from 1 to 8; and

y is an integer from 1-10. In some embodiments, R¹⁶ is

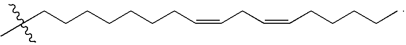

In some embodiments, at least one of the double bonds has an E configuration. In some embodiments, both of the double bonds have an E configuration. In some embodiments, R¹⁶ has the following formula:

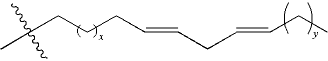

wherein

x is an integer from 1 to 8; and

y is an integer from 1-10. In some embodiments, R¹⁶ has three double bond moieties. In some embodiments, at least one of the double bonds has a Z configuration. In some embodiments, at least two of the double bonds have a Z configuration. In some embodiments, all three of the double bonds have a Z configuration. In some embodiments, R¹⁶ has the following formula:

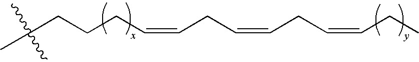

wherein

x is an integer from 1 to 8; and

y is an integer from 1-10. In some embodiments, at least one of the double bonds has an E configuration. In some embodiments, at least two of the double bonds have an E configuration. In some embodiments, all three of the double bonds have an E configuration. In some embodiments, R¹⁶ has the following formula:

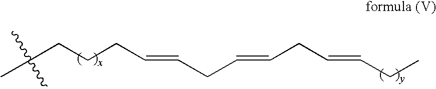

wherein

x is an integer from 1 to 8; and

y is an integer from 1-10.

In some embodiments, R¹⁶ is alkynyl.

In some embodiments, R¹⁶ is R^{d} or C₁-C₁₀ alkyl substituted with NHC(O)R^{d}. In some embodiments, R¹⁶ is R^{d}. In some embodiments, R¹⁶ is R^{d} and R^{d} is an unsubstituted cholesterol moiety. In some embodiments, R¹⁶ is C₁-C₁₀ alkyl substituted with NHC(O)R^{d}. In some embodiments, R^{d} is an unsubstituted cholesterol moiety. In some embodiments, R¹⁶ is (CH₂)₅NHC(O)R^{d}, and R^{d} is an unsubstituted cholesterol moiety. In some embodiments, R¹⁶ is a cholesterol moiety, substituted with C(O)OR^{L}, C(O)NR^{L}R^{L′}, R^{L}, S(O)ₘR^{L}, or S(O)ₘNR^{L}R^{L′}. In some embodiments, R¹⁶ is a cholesterol moiety, substituted with C(O)NR^{L}R^{L′}. In some embodiments, R^{L} is alkenyl and R^{L′} is H. In some embodiments, R^{L} has a Z configuration. In some embodiments, R^{L} is C¹⁸ alkenyl.

In some embodiments, R³ is NR¹⁴C(O)R¹⁶ and wherein R⁴ is H.

In some embodiments, R⁵ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙ NR¹⁴C(O)R¹⁶, (CH₂)ₙO—N═CR¹⁶; (CH₂)N—N═CR¹⁶;

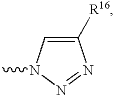

where n is 0 or 1. In some embodiments, R⁶ is H.

In some embodiments, R⁵ is C(O)OR¹³ or C(O)NR¹⁴R¹⁵. In some embodiments, R⁶ is H.

In some embodiments, R⁵ is C(O)NR¹⁴R¹⁵.

In some embodiments, R⁵ is C(O)NR¹⁴R¹⁵ and R⁶ is H.

In some embodiments, R¹⁴ is H.

In some embodiments, R¹⁵ is alkyl optionally substituted with 1-3 nitrogen containing moieties selected from the group consisting of NR¹⁸R¹⁹ or a nitrogen containing heterocyclyl.

In some embodiments, R¹⁵ is alkyl optionally substituted with 1-3 nitrogen containing moieties selected from the group consisting of NR¹⁸R¹⁹ or a nitrogen containing heterocyclyl and R¹⁴ is H.

In some embodiments, R¹⁵ is C₁-C₆ alkyl substituted with a nitrogen containing moiety selected from the group consisting of NR¹⁸R¹⁹ or a nitrogen containing heterocyclyl.

In some embodiments, R¹⁵ is C₁-C₆ alkyl substituted with a nitrogen containing moiety selected from the group consisting of NR¹⁸R¹⁹ or a nitrogen containing heterocyclyl and R¹⁴ is H.

In some embodiments, R¹⁵ is C₁-C₆ alkyl substituted NR¹⁸R¹⁹. In some embodiments, R¹⁸ and R¹⁹ are both alkyl. In some embodiments, R¹⁸ and R¹⁹ are both C₁-C₆ alkyl. In some embodiments, R¹⁸ and R¹⁹ are both methyl.

In some embodiments, wherein R¹⁵ is

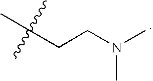

In some embodiments, R¹⁴ is H.

In some embodiments, R¹⁵ is C₁-C₆ alkyl substituted with a nitrogen containing heterocyclyl. In some embodiments, the nitrogen containing heterocyclyl has 2 ring nitrogens. In some embodiments, the nitrogen containing heteroaryl has 2 ring nitrogens. In some embodiments, heteroaryl is an imidazolyl. In some embodiments, R¹⁵ is

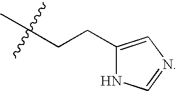

In some embodiments, both R¹⁴ and R¹⁵ are C₁-C₆ alkyl substituted NR¹⁸R¹⁹. In some embodiments, both R¹⁴ and R¹⁵ are

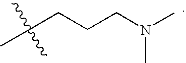

In some embodiments, one or both of R¹⁴ and R¹⁵ are alkyl. In some embodiments, one or both of R¹⁴ and R¹⁵ is C₁₀₋₃₀ alkyl. In some embodiments, one or both of R¹⁴ and R¹⁵ is C₁₀₋₁₈ alkyl. In some embodiments, one or both of R¹⁴ and R¹⁵ is C₁₂ alkyl.

In some embodiments, one or both of R¹⁴ and R¹⁵ is alkenyl. In some embodiments, one or both of R¹⁴ and R¹⁵ is C₆-C₃₀ alkenyl. In some embodiments, one or both of R¹⁴ and R¹⁵ has a single double bond. In some embodiments, the double bond has a Z configuration. In some embodiments, one or both of R¹⁴ and R¹⁵ has two double bonds. In some embodiments, at least one of the double bonds have a Z configuration. In some embodiments, both of the double bonds have a Z configuration. In some embodiments, one or both of R¹⁴ and R¹⁵ has the following formula:

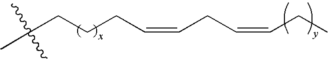

wherein

x is an integer from 1 to 8; and

y is an integer from 1-10. one or both of R¹⁴ and R¹⁵ is

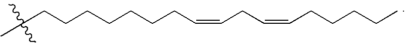

In some embodiments, at least one of the double bonds has an E configuration. In some embodiments, both of the double bonds have an E configuration. In some embodiments, one or both of R¹⁴ and R¹⁵ has the following formula:

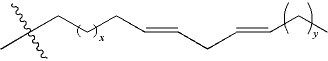

wherein

x is an integer from 1 to 8; and

y is an integer from 1-10. In some embodiments, one or both of R¹⁴ and R¹⁵ has three double bond moieties. In some embodiments, at least one of the double bonds has a Z configuration. In some embodiments, at least two of the double bonds have a Z configuration. In some embodiments, all three of the double bonds have a Z configuration. In some embodiments, one or both of R¹⁴ and R¹⁵ has the following formula:

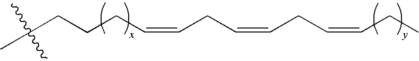

wherein

x is an integer from 1 to 8; and

y is an integer from 1-10. In some embodiments, at least one of the double bonds have an E configuration. In some embodiments, at least two of the double bonds have an E configuration. In some embodiments, all three of the double bonds have an E configuration. In some embodiments, one or both of R¹⁴ and R¹⁵ has the following formula:

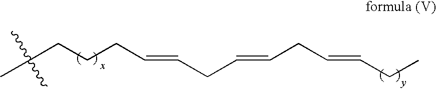

wherein

x is an integer from 1 to 8; and

y is an integer from 1-10.

In some embodiments, one or both of R¹⁴ and R¹⁵ is alkynyl.

In some embodiments, the compound of formula (III) is present in a diastereomeric mixture (for example, having at least one of the carbons at which R³ or R⁵ is attached being an asymmetric carbon, for having both of the carbons at which R³ or R⁵ is attached being an asymmetric carbon).

In some embodiments, the compound of formula (III) has at least a 60% diastereomeric excess of the 2R,4R configuration (e.g., at least about 65%, at least about 70%, at least about 75%, at least about 80%, at least about 85%, at least about 90%, at least about 95%, at least about 98%, at least about 99% diastereomeric excess of the 2R,4R configuration). In some embodiments, the compound of formula (III) is a substantially pure form of the 2R,4R configuration.

In some embodiments, the compound of formula (III) has at least a 60% diastereomeric excess of the 2S,4R configuration (e.g., at least about 65%, at least about 70%, at least about 75%, at least about 80%, at least about 85%, at least about 90%, at least about 95%, at least about 98%, at least about 99% diastereomeric excess of the 2S,4R configuration). In some embodiments, the compound of formula (III) is a substantially pure form of the 2S,4R configuration.

In some embodiments, the compound of formula (III) has at least a 60% diastereomeric excess of the 2S,4S configuration (e.g., at least about 65%, at least about 70%, at least about 75%, at least about 80%, at least about 85%, at least about 90%, at least about 95%, at least about 98%, at least about 99% diastereomeric excess of the 2S,4S configuration). In some embodiments, the compound of formula (III) is a substantially pure form of the 2S,4S configuration.

In some embodiments, the compound of formula (III) has at least a 60% diastereomeric excess of the 2R,4S configuration (e.g., at least about 65%, at least about 70%, at least about 75%, at least about 80%, at least about 85%, at least about 90%, at least about 95%, at least about 98%, at least about 99% diastereomeric excess of the 2R,4S configuration). In some embodiments, the compound of formula (III) is a substantially pure form of the 2R,4S configuration.

In some embodiments, the compound has a formula (III′)

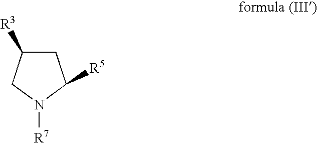

wherein,

each of R³ and R⁵ is, independently H, (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—S—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹⁶, (CH₂)ₙNR¹⁴C(O)R¹⁶, (CH₂)ₙ O—N═CR¹⁶; (CH₂)N—N═CR¹⁶; (CH₂)ₙNR¹⁴SO₂R¹⁶, (CH₂)ₙCH═N—OR¹⁶, (CH₂)ₙCH═N—NR¹⁴R¹⁶, C₁-C₃₀ alkyl, C₂-C₃₀ alkenyl, C₂-C₃₀ alkynyl, heterocycle or heteroaryl (e.g. triazole)

each R⁷ and R⁸ is independently H, C₁-C₆ alkyl, SO₂R¹⁶ or a nitrogen protecting group, e.g., a C(O)Oalkyl moiety such as BOC, or C(O)R¹⁶;

R¹³, for each occurrence, is independently H, alkyl alkenyl, or alkynyl;

each R¹⁴ and R¹⁵, for each occurrence, is independently H, alkyl alkenyl, or alkynyl, each of which is optionally substituted with 1-3 nitrogen containing moieties selected from the group consisting of NR¹⁸R¹⁹ or a nitrogen containing heterocyclyl;

R¹⁶, for each occurrence, is alkyl alkenyl, alkynyl, R^{d} or C₁-C₁₀ alkyl substituted with NHC(O)R^{d} or with 1-3 nitrogen containing moieties selected from the group consisting of NR¹⁸R¹⁹ or a nitrogen containing heterocyclyl;

R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};

each R^{L} and R^{L′} is independently H, alkyl alkenyl, or alkynyl;

each R¹⁸ and R¹⁹, for each occurrence, is independently, H, alkyl alkenyl, alkynyl, or a nitrogen protecting group such as BOC, Fmoc or benzyl;

m is 0, 1, or 2

n is an integer from 1 to 4.

In some embodiments, R⁷ is H.

In some embodiments, R⁷ is a nitrogen protecting group, for example BOC.

In some embodiments, R⁷ is C(O)R¹⁶.

In one embodiment, R⁷ is SO₂R¹⁶.

In some embodiments, R¹⁶ is alkyl substituted with 1-3 NR¹⁸R¹⁹, for example, R¹⁶ is alkyl substituted with 2 NR¹⁸R¹⁹. In some embodiments, each NR¹⁸R¹⁹ is NH₂. In some embodiments, one NR¹⁸R¹⁹ is NH₂. In some embodiments, one NR¹⁸R¹⁹ is NMe₂. In some embodiments, R¹⁸ is H and R¹⁹ is Me of each NR¹⁸R¹⁹. In some embodiments, R¹⁸ is H and R¹⁹ is Me of one NR¹⁸R¹⁹ and R¹⁸ and R¹⁹ is H for the second NR¹⁸R¹⁹. In some embodiments, R¹⁸ is H and R¹⁹ is Me of one NR¹⁸R¹⁹ and R¹⁸ and R¹⁹ is Me for the second NR¹⁸R¹⁹. In some embodiments, R¹⁶ is alkyl substituted with NH₂ and NMe₂.

In some embodiments, R¹⁶ is substituted with a nitrogen containing heterocyclyl. In some embodiments, R¹⁶ is further substituted by NR¹⁸R¹⁹. In some embodiments, wherein NR¹⁸R¹⁹ is NH₂. In some embodiments, the nitrogen containing heterocyclyl has 2 ring nitrogens. In some embodiments, the nitrogen containing heterocyclcyl is a nitrogen containing heteroaryl. In some embodiments, the nitrogen containing heteroaryl has 2 ring nitrogens. In some embodiments, the heteroaryl is an imidazolyl.

In some embodiments, R¹⁶ is alkyl substituted with NH₂ and imidazolyl. In some embodiments, R¹⁶ is

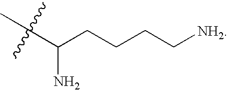

In some embodiments, R¹⁶ is

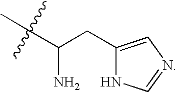

In some embodiments, R³ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙ O—N═CR¹⁶; (CH₂)N—N═CR¹⁶;

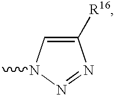

where n is 0 or 1. In some embodiments, R⁴ is H.

In some embodiments, R³ is OR¹³, NR¹⁴R¹⁵, C(O)NR¹⁴R¹⁵, or NR¹⁴C(O)R¹⁶.

In some embodiments, R³ is OR¹³, NR¹⁴R¹⁵, C(O)NR¹⁴R¹⁵, or NR¹⁴C(O)R¹⁶; and wherein R⁴ is H.

In some embodiments, R³ is NR¹⁴R¹⁵ or NR¹⁴C(O)R¹⁶.

In some embodiments, R³ is NR¹⁴R¹⁵ or NR¹⁴C(O)R¹⁶ and R⁴ is H.

In some embodiments, R³ is NR¹⁴C(O)R¹⁶.

In some embodiments, R¹⁶ is alkyl, for example, R¹⁶ is Cl₁₀₋₃₀ alkyl, R¹⁶ is C₁₀₋₁₈ alkyl, or R¹⁶ is C₁₅ alkyl.

In some embodiments, R¹⁶ is alkenyl. In some embodiments, R¹⁶ is C₆-C₃₀ alkenyl. In some embodiments, R¹⁶ has a single double bond. In some embodiments, the double bond has a Z configuration. In some embodiments, R¹⁶ has two double bonds. In some embodiments, at least one of the double bonds has a Z configuration. In some embodiments, both of the double bonds have a Z configuration. In some embodiments, R¹⁶ has the following formula:

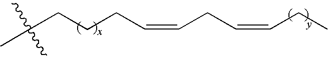

wherein

x is an integer from 1 to 8; and

y is an integer from 1-10. In some embodiments, R¹⁶ is

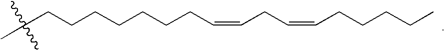

In some embodiments, at least one of the double bonds has an E configuration. In some embodiments, both of the double bonds have an E configuration. In some embodiments, R¹⁶ has the following formula:

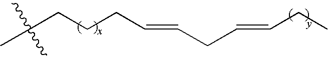

wherein

x is an integer from 1 to 8; and

y is an integer from 1-10. In some embodiments, R¹⁶ has three double bond moieties. In some embodiments, at least one of the double bonds has a Z configuration. In some embodiments, at least two of the double bonds have a Z configuration. In some embodiments, all three of the double bonds have a Z configuration. In some embodiments, R¹⁶ has the following formula:

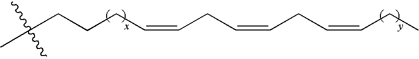

wherein

x is an integer from 1 to 8; and

y is an integer from 1-10. In some embodiments, at least one of the double bonds has an E configuration. In some embodiments, at least two of the double bonds have an E configuration. In some embodiments, all three of the double bonds have an E configuration. In some embodiments, R¹⁶ has the following formula:

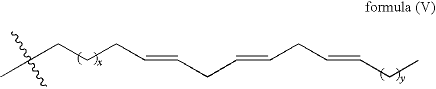

wherein

x is an integer from 1 to 8; and

y is an integer from 1-10.

In some embodiments, R¹⁶ is alkynyl.

In some embodiments, R¹⁶ is R^{d} or C₁-C₁₀ alkyl substituted with NHC(O)R^{d}. In some embodiments, R¹⁶ is R^{d}. In some embodiments, R¹⁶ is R^{d} and R^{d} is an unsubstituted cholesterol moiety. In some embodiments, R¹ is C₁-C₁₀ alkyl substituted with NHC(O)R^{d}. In some embodiments, R^{d} is an unsubstituted cholesterol moiety. In some embodiments, R¹⁶ is (CH₂)₅NHC(O)R^{d}, and R^{d} is an unsubstituted cholesterol moiety. In some embodiments, R¹⁶ is a cholesterol moiety, substituted with C(O)OR^{L}, C(O)NR^{L}R^{L′}, R^{L}, S(O)ₘR^{L}, or S(O)ₘNR^{L}R^{L′}. In some embodiments, R¹⁶ is a cholesterol moiety, substituted with C(O)NR^{L}R^{L′}. In some embodiments, R^{L} is alkenyl and R^{L′} is H. In some embodiments, R^{L} has a Z configuration. In some embodiments, R^{L} is C¹⁸ alkenyl.

In some embodiments, R³ is NR¹⁴C(O)R¹⁶ and wherein R⁴ is H.

In some embodiments, R⁵ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙ O—N═CR¹⁶; (CH₂)N—N═CR¹⁶;

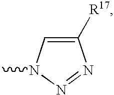

where n is 0 or 1. In some embodiments, R⁶ is H.

In some embodiments, R⁵ is C(O)OR¹³ or C(O)NR¹⁴R¹⁵. In some embodiments, R⁶ is H.

In some embodiments, R⁵ is C(O)NR¹⁴R¹⁵.

In some embodiments, R⁵ is C(O)NR¹⁴R¹⁵ and R⁶ is H.

In some embodiments, R¹⁴ is H.

In some embodiments, R¹⁵ is alkyl optionally substituted with 1-3 nitrogen containing moieties selected from the group consisting of NR¹⁸R¹⁹ or a nitrogen containing heterocyclyl.

In some embodiments, R¹⁵ is alkyl optionally substituted with 1-3 nitrogen containing moieties selected from the group consisting of NR¹⁸R¹⁹ or a nitrogen containing heterocyclyl and R¹⁴ is H.

In some embodiments, R¹⁵ is C₁-C₆ alkyl substituted with a nitrogen containing moiety selected from the group consisting of NR¹⁸R¹⁹ or a nitrogen containing heterocyclyl.

In some embodiments, R¹⁵ is C₁-C₆ alkyl substituted with a nitrogen containing moiety selected from the group consisting of NR¹⁸R¹⁹ or a nitrogen containing heterocyclyl and R¹⁴ is H.

In some embodiments, R¹⁵ is C₁-C₆ alkyl substituted NR¹⁸R¹⁹. In some embodiments, R¹⁸ and R¹⁹ are both alkyl. In some embodiments, R¹⁸ and R¹⁹ are both C₁-C₆ alkyl. In some embodiments, R¹⁸ and R¹⁹ are both methyl.

In some embodiments, wherein R¹⁵ is

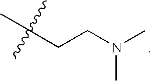

In some embodiments, R¹⁴ is H.

In some embodiments, R¹⁵ is C₁-C₆ alkyl substituted with a nitrogen containing heterocyclyl. In some embodiments, the nitrogen containing heterocyclyl has 2 ring nitrogens. In some embodiments, the nitrogen containing heteroaryl has 2 ring nitrogens. In some embodiments, heteroaryl is an imidazolyl. In some embodiments, R¹⁵ is

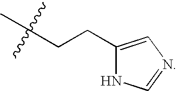

In some embodiments, both R¹⁴ and R¹⁵ are C₁-C₆ alkyl substituted NR¹⁸R¹⁹. In some embodiments, both R¹⁴ and R¹⁵ are

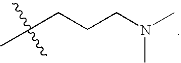

In some embodiments, one or both of R¹⁴ and R¹⁵ are alkyl. In some embodiments, one or both of R¹⁴ and R¹⁵ is C₁₀₋₃₀ alkyl. In some embodiments, one or both of R¹⁴ and R¹⁵ is C₁₀₋₁₈ alkyl. In some embodiments, one or both of R¹⁴ and R¹⁵ is C₁₂ alkyl.

In some embodiments, one or both of R¹⁴ and R¹⁵ is alkenyl. In some embodiments, one or both of R¹⁴ and R¹⁵ is C₆-C₃₀ alkenyl. In some embodiments, one or both of R¹⁴ and R¹⁵ has a single double bond. In some embodiments, the double bond has a Z configuration. In some embodiments, one or both of R¹⁴ and R¹⁵ has two double bonds. In some embodiments, at least one of the double bonds have a Z configuration. In some embodiments, both of the double bonds have a Z configuration. In some embodiments, one or both of R¹⁴ and R¹⁵ has the following formula:

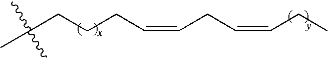

wherein

x is an integer from 1 to 8; and

y is an integer from 1-10. one or both of R¹⁴ and R¹⁵ is

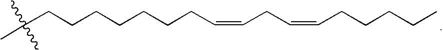

In some embodiments, at least one of the double bonds has an E configuration. In some embodiments, both of the double bonds have an E configuration. In some embodiments, one or both of R¹⁴ and R¹⁵ has the following formula:

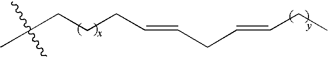

wherein

x is an integer from 1 to 8; and

y is an integer from 1-10. In some embodiments, one or both of R¹⁴ and R¹⁵ has three double bond moieties. In some embodiments, at least one of the double bonds has a Z configuration. In some embodiments, at least two of the double bonds have a Z configuration. In some embodiments, all three of the double bonds have a Z configuration. In some embodiments, one or both of R¹⁴ and R¹⁵ has the following formula:

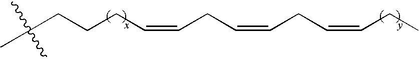

wherein

x is an integer from 1 to 8; and

y is an integer from 1-10. In some embodiments, at least one of the double bonds have an E configuration. In some embodiments, at least two of the double bonds have an E configuration. In some embodiments, all three of the double bonds have an E configuration. In some embodiments, one or both of R¹⁴ and R¹⁵ has the following formula:

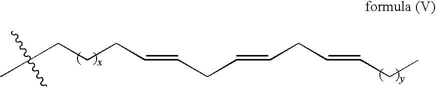

wherein

x is an integer from 1 to 8; and

y is an integer from 1-10.

In some embodiments, one or both of R¹⁴ and R¹⁵ is alkynyl.

In one aspect, the invention features a compound of formula (IV)

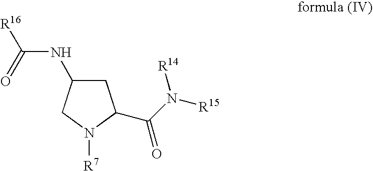

wherein,

each R⁷H, C₁-C₆ alkyl, a nitrogen protecting group, e.g., a C(O)Oalkyl moiety such as BOC, or C(O)R¹⁶;

each R¹⁴ and R¹⁵, for each occurrence, is independently H, alkyl alkenyl, or alkynyl, each of which is optionally substituted with 1-3 nitrogen containing moieties selected from the group consisting of NR¹⁸R¹⁹ or a nitrogen containing heterocyclyl;

R¹⁶, for each occurrence, is alkyl alkenyl, alkynyl, R^{d} or C₁-C₁₀ alkyl substituted with NHC(O)R^{d} or with 1-3 nitrogen containing moieties selected from the group consisting of NR¹⁸R¹⁹ or a nitrogen containing heterocyclyl;

R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};

each R^{L} and R^{L′} is independently H, alkyl alkenyl, or alkynyl;

m is 0, 1, or 2

n is an integer from 1 to 4.

In some embodiments, R⁷ is H.

In some embodiments, R⁷ is a nitrogen protecting group, for example BOC.

In some embodiments, R⁷ is C(O)R¹⁶.

In some embodiments, R¹⁶ is alkyl substituted with 1-3 NR¹⁸R¹⁹, for example, R¹⁶ is alkyl substituted with 2 NR¹⁸R¹⁹. In some embodiments, each NR¹⁸R¹⁹ is NH₂. In some embodiments, one NR¹⁸R¹⁹ is NH₂. In some embodiments, one NR¹⁸R¹⁹ is NMe₂. In some embodiments, R¹⁸ is H and R¹⁹ is Me of each NR¹⁸R¹⁹. In some embodiments, R¹⁸ is H and R¹⁹ is Me of one NR¹⁸R¹⁹ and R¹⁸ and R¹⁹ is H for the second NR¹⁸R¹⁹. In some embodiments, R¹⁸ is H and R¹⁹ is Me of one NR¹⁸R¹⁹ and R¹⁸ and R¹⁹ is Me for the second NR¹⁸R¹⁹. In some embodiments, R¹⁶ is alkyl substituted with NH₂ and NMe₂.

In some embodiments, R¹⁶ is substituted with a nitrogen containing heterocyclyl. In some embodiments, R¹⁶ is further substituted by NR¹⁸R¹⁹. In some embodiments, wherein NR¹⁸R¹⁹ is NH₂. In some embodiments, the nitrogen containing heterocyclyl has 2 ring nitrogens. In some embodiments, the nitrogen containing heterocyclcyl is a nitrogen containing heteroaryl. In some embodiments, the nitrogen containing heteroaryl has 2 ring nitrogens. In some embodiments, the heteroaryl is an imidazolyl.

In some embodiments, R¹⁶ is alkyl substituted with NH₂ and imidazolyl. In some embodiments, R¹⁶ is

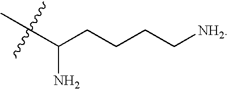

In some embodiments, R¹⁶ is

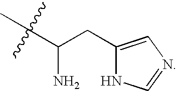

In some embodiments, R³ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙ O—N═CR¹⁶; (CH₂)N—N═CR¹⁶;

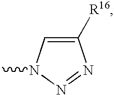

where n is 0 or 1. In some embodiments, R⁴ is H.

In some embodiments, R¹⁶ is alkyl, for example, R¹⁶ is C₁₀₋₃₀ alkyl, R¹⁶ is C₁₀₋₁₈ alkyl, or R¹⁶ is C₁₅alkyl.

In some embodiments, R¹⁶ is alkenyl. In some embodiments, R¹⁶ is C₆-C₃₀ alkenyl. In some embodiments, R¹⁶ has a single double bond. In some embodiments, the double bond has a Z configuration. In some embodiments, R¹⁶ has two double bonds. In some embodiments, at least one of the double bonds has a Z configuration. In some embodiments, both of the double bonds have a Z configuration. In some embodiments, R¹⁶ has the following formula:

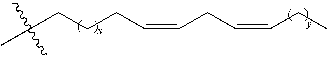

wherein

x is an integer from 1 to 8; and

y is an integer from 1-10. In some embodiments, R¹⁶ is

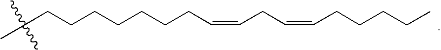

In some embodiments, at least one of the double bonds has an E configuration. In some embodiments, both of the double bonds have an E configuration. In some embodiments, R¹⁶ has the following formula:

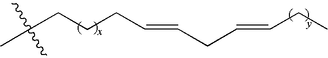

wherein

x is an integer from 1 to 8; and

y is an integer from 1-10. In some embodiments, R¹⁶ has three double bond moieties. In some embodiments, at least one of the double bonds has a Z configuration. In some embodiments, at least two of the double bonds have a Z configuration. In some embodiments, all three of the double bonds have a Z configuration. In some embodiments, R¹⁶ has the following formula:

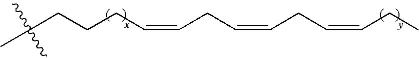

wherein

x is an integer from 1 to 8; and

y is an integer from 1-10. In some embodiments, at least one of the double bonds has an E configuration. In some embodiments, at least two of the double bonds have an E configuration. In some embodiments, all three of the double bonds have an E configuration. In some embodiments, R¹⁶ has the following formula:

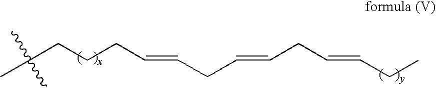

wherein

x is an integer from 1 to 8; and

y is an integer from 1-10.

In some embodiments, R¹⁶ is alkynyl.

In some embodiments, R¹⁶ is R^{d} or C₁-C₁₀ alkyl substituted with NHC(O)R^{d}. In some embodiments, R¹⁶ is R^{d}. In some embodiments, R¹⁶ is R^{d} and R^{d} is an unsubstituted cholesterol moiety. In some embodiments, R¹⁶ is C₁-C₁₀ alkyl substituted with NHC(O)R^{d}. In some embodiments, R^{d} is an unsubstituted cholesterol moiety. In some embodiments, R¹⁶ is (CH₂)₅NHC(O)R^{d}, and R^{d} is an unsubstituted cholesterol moiety. In some embodiments, R¹⁶ is a cholesterol moiety, substituted with C(O)OR^{L}, C(O)NR^{L}R^{L′}, R^{L}, S(O)ₘR^{L}, or S(O)ₘNR^{L}R^{L′}. In some embodiments, R¹⁶ is a cholesterol moiety, substituted with C(O)NR^{L}R^{L′}. In some embodiments, R^{L} is alkenyl and R^{L′} is H. In some embodiments, R^{L} has a Z configuration. In some embodiments, R^{L} is C¹⁸ alkenyl.

In some embodiments, R¹⁴ is H.

In some embodiments, R¹⁵ is alkyl optionally substituted with 1-3 nitrogen containing moieties selected from the group consisting of NR¹⁸R¹⁹ or a nitrogen containing heterocyclyl.

In some embodiments, R¹⁵ is alkyl optionally substituted with 1-3 nitrogen containing moieties selected from the group consisting of NR¹⁸R¹⁹ or a nitrogen containing heterocyclyl and R¹⁴ is H.

In some embodiments, R¹⁵ is C₁-C₆ alkyl substituted with a nitrogen containing moiety selected from the group consisting of NR¹⁸R¹⁹ or a nitrogen containing heterocyclyl.

In some embodiments, R¹⁵ is C₁-C₆ alkyl substituted with a nitrogen containing moiety selected from the group consisting of NR¹⁸R¹⁹ or a nitrogen containing heterocyclyl and R¹⁴ is H.

In some embodiments, R¹⁵ is C₁-C₆ alkyl substituted NR¹⁸R¹⁹. In some embodiments, R¹⁸ and R¹⁹ are both alkyl. In some embodiments, R¹⁸ and R¹⁹ are both C₁-C₆ alkyl. In some embodiments, R¹⁸ and R¹⁹ are both methyl.

In some embodiments, wherein R¹⁵ is

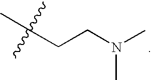

In some embodiments, R¹⁴ is H.

In some embodiments, R¹⁵ is C₁-C₆ alkyl substituted with a nitrogen containing heterocyclyl. In some embodiments, the nitrogen containing heterocyclyl has 2 ring nitrogens. In some embodiments, the nitrogen containing heteroaryl has 2 ring nitrogens. In some embodiments, heteroaryl is an imidazolyl. In some embodiments, R¹⁵ is

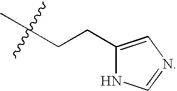

In some embodiments, both R¹⁴ and R¹⁵ are C₁-C₆ alkyl substituted NR¹⁸R¹⁹. In some embodiments, both R¹⁴ and R¹⁵ are

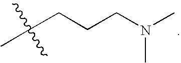

In some embodiments, one or both of R¹⁴ and R¹⁵ are alkyl. In some embodiments, one or both of R¹⁴ and R¹⁵ is C₁₀₋₃₀ alkyl. In some embodiments, one or both of R¹⁴ and R¹⁵ is C₁₀₋₁₈ alkyl. In some embodiments, one or both of R¹⁴ and R¹⁵ is C₁₂ alkyl.

In some embodiments, one or both of R¹⁴ and R¹⁵ is alkenyl. In some embodiments, one or both of R¹⁴ and R¹⁵ is C₆-C₃₀ alkenyl. In some embodiments, one or both of R¹⁴ and R¹⁵ has a single double bond. In some embodiments, the double bond has a Z configuration. In some embodiments, one or both of R¹⁴ and R¹⁵ has two double bonds. In some embodiments, at least one of the double bonds have a Z configuration. In some embodiments, both of the double bonds have a Z configuration. In some embodiments, one or both of R¹⁴ and R¹⁵ has the following formula:

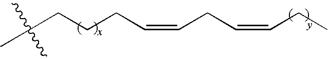

wherein

x is an integer from 1 to 8; and

y is an integer from 1-10. one or both of R¹⁴ and R¹⁵ is

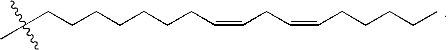

In some embodiments, at least one of the double bonds has an E configuration. In some embodiments, both of the double bonds have an E configuration. In some embodiments, one or both of R¹⁴ and R¹⁵ has the following formula:

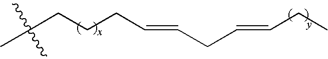

wherein

x is an integer from 1 to 8; and

y is an integer from 1-10. In some embodiments, one or both of R¹⁴ and R¹⁵ has three double bond moieties. In some embodiments, at least one of the double bonds has a Z configuration. In some embodiments, at least two of the double bonds have a Z configuration. In some embodiments, all three of the double bonds have a Z configuration. In some embodiments, one or both of R¹⁴ and R¹⁵ has the following formula:

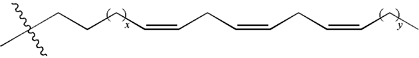

wherein

x is an integer from 1 to 8; and

y is an integer from 1-10. In some embodiments, at least one of the double bonds have an E configuration. In some embodiments, at least two of the double bonds have an E configuration. In some embodiments, all three of the double bonds have an E configuration. In some embodiments, one or both of R¹⁴ and R¹⁵ has the following formula:

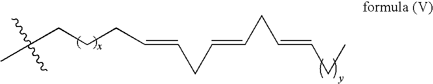

wherein

x is an integer from 1 to 8; and

y is an integer from 1-10.

In some embodiments, one or both of R¹⁴ and R¹⁵ is alkynyl.

The compound of claim x, wherein the compound of formula (IV) is present in a diastereomeric mixture (for example, having at least one of the carbons at which R³ or R⁵ is attached being an asymmetric carbon, for having both of the carbons at which R³ or R⁵ is attached being an asymmetric carbon).

In some embodiments, the compound of formula (IV) has at least a 60% diastereomeric excess of the 2R,4R configuration (e.g., at least about 65%, at least about 70%, at least about 75%, at least about 80%, at least about 85%, at least about 90%, at least about 95%, at least about 98%, at least about 99% diastereomeric excess of the 2R,4R configuration). In some embodiments, the compound of formula (IV) is a substantially pure form of the 2R,4R configuration.

In some embodiments, the compound of formula (IV) has at least a 60% diastereomeric excess of the 2S,4R configuration (e.g., at least about 65%, at least about 70%, at least about 75%, at least about 80%, at least about 85%, at least about 90%, at least about 95%, at least about 98%, at least about 99% diastereomeric excess of the 2S,4R configuration). In some embodiments, the compound of formula (IV) is a substantially pure form of the 2S,4R configuration.

In some embodiments, the compound of formula (IV) has at least a 60% diastereomeric excess of the 2S,4S configuration (e.g., at least about 65%, at least about 70%, at least about 75%, at least about 80%, at least about 85%, at least about 90%, at least about 95%, at least about 98%, at least about 99% diastereomeric excess of the 2S,4S configuration). In some embodiments, the compound of formula (IV) is a substantially pure form of the 2S,4S configuration.

In some embodiments, the compound of formula (IV) has at least a 60% diastereomeric excess of the 2R,4S configuration (e.g., at least about 65%, at least about 70%, at least about 75%, at least about 80%, at least about 85%, at least about 90%, at least about 95%, at least about 98%, at least about 99% diastereomeric excess of the 2R,4S configuration). In some embodiments, the compound of formula (IV) is a substantially pure form of the 2R,4S configuration.

In some embodiments, formula (IV′)

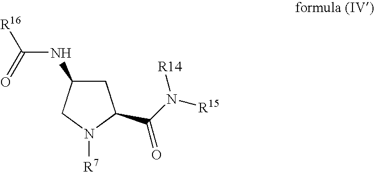

each R⁷H, C₁-C₆ alkyl, a nitrogen protecting group, e.g., a C(O)Oalkyl moiety such as BOC, or C(O)R¹⁶;

each R¹⁴ and R¹⁵, for each occurrence, is independently H, alkyl alkenyl, or alkynyl, each of which is optionally substituted with 1-3 nitrogen containing moieties selected from the group consisting of NR¹⁸R¹⁹ or a nitrogen containing heterocyclyl;

R¹⁶, for each occurrence, is alkyl alkenyl, alkynyl, R^{d} or C₁-C₁₀ alkyl substituted with NHC(O)R^{d};

R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};

each R^{L} and R^{L′} is independently H, alkyl alkenyl, or alkynyl;

n is an integer from 1 to 4.

In some embodiments, R⁷ is H.

In some embodiments, R⁷ is a nitrogen protecting group, for example BOC.

In some embodiments, R⁷ is C(O)R¹⁶.

In some embodiments, R¹⁶ is alkyl substituted with 1-3 NR¹⁸R¹⁹, for example, R¹⁶ is alkyl substituted with 2 NR¹⁸R¹⁹. In some embodiments, each NR¹⁸R¹⁹ is NH₂. In some embodiments, one NR¹⁸R¹⁹ is NH₂. In some embodiments, one NR¹⁸R¹⁹ is NMe₂. In some embodiments, R¹⁸ is H and R¹⁹ is Me of each NR¹⁸R¹⁹. In some embodiments, R¹⁸ is H and R¹⁹ is Me of one NR¹⁸R¹⁹ and R¹⁸ and R¹⁹ is H for the second NR¹⁸R¹⁹. In some embodiments, R¹⁸ is H and R¹⁹ is Me of one NR¹⁸R¹⁹ and R¹⁸ and R¹⁹ is Me for the second NR¹⁸R¹⁹. In some embodiments, R¹⁶ is alkyl substituted with NH₂ and NMe₂.

In some embodiments, R¹⁶ is substituted with a nitrogen containing heterocyclyl. In some embodiments, R¹⁶ is further substituted by NR¹⁸R¹⁹. In some embodiments, wherein NR¹⁸R¹⁹ is NH₂. In some embodiments, the nitrogen containing heterocyclyl has 2 ring nitrogens. In some embodiments, the nitrogen containing heterocyclcyl is a nitrogen containing heteroaryl. In some embodiments, the nitrogen containing heteroaryl has 2 ring nitrogens. In some embodiments, the heteroaryl is an imidazolyl.

In some embodiments, R¹⁶ is alkyl substituted with NH₂ and imidazolyl. In some embodiments, R¹⁶ is

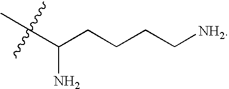

In some embodiments, R¹⁶ is

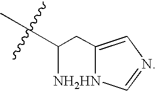

In some embodiments, R³ is (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙ O—N═CR¹⁶; (CH₂)N—N═CR¹⁶;

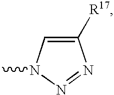

where n is 0 or 1. In some embodiments, R⁴ is H.

In some embodiments, R¹⁶ is alkyl, for example, R¹⁶ is C₁₀₋₃₀ alkyl, R¹⁶ is C₁₀₋₁₈ alkyl, or R¹⁶ is C₁₅ alkyl.

In some embodiments, R¹⁶ is alkenyl. In some embodiments, R¹⁶ is C₆-C₃₀ alkenyl. In some embodiments, R¹⁶ has a single double bond. In some embodiments, the double bond has a Z configuration. In some embodiments, R¹⁶ has two double bonds. In some embodiments, at least one of the double bonds has a Z configuration. In some embodiments, both of the double bonds have a Z configuration. In some embodiments, R¹⁶ has the following formula:

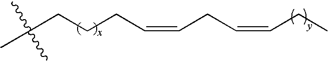

wherein

x is an integer from 1 to 8; and

y is an integer from 1-10. In some embodiments, R¹⁶ is

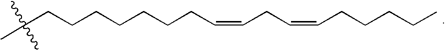

In some embodiments, at least one of the double bonds has an E configuration. In some embodiments, both of the double bonds have an E configuration. In some embodiments, R¹⁶ has the following formula:

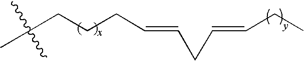

wherein

x is an integer from 1 to 8; and

y is an integer from 1-10. In some embodiments, R¹⁶ has three double bond moieties. In some embodiments, at least one of the double bonds has a Z configuration. In some embodiments, at least two of the double bonds have a Z configuration. In some embodiments, all three of the double bonds have a Z configuration. In some embodiments, R¹⁶ has the following formula:

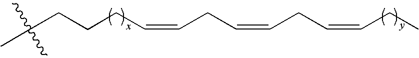

wherein

x is an integer from 1 to 8; and

y is an integer from 1-10. In some embodiments, at least one of the double bonds has an E configuration. In some embodiments, at least two of the double bonds have an E configuration. In some embodiments, all three of the double bonds have an E configuration. In some embodiments, R¹⁶ has the following formula:

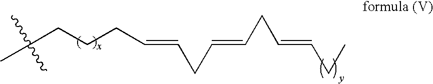

wherein

x is an integer from 1 to 8; and

y is an integer from 1-10.

In some embodiments, R¹⁶ is alkynyl.

In some embodiments, R¹⁶ is R^{d} or C₁-C₁₀ alkyl substituted with NHC(O)R^{d}. In some embodiments, R¹⁶ is R^{d}. In some embodiments, R¹⁶ is R^{d} and R^{d} is an unsubstituted cholesterol moiety. In some embodiments, R¹⁶ is C₁-C₁₀ alkyl substituted with NHC(O)R^{d}. In some embodiments, R^{d} is an unsubstituted cholesterol moiety. In some embodiments, R¹⁶ is (CH₂)₅NHC(O)R^{d}, and R^{d} is an unsubstituted cholesterol moiety. In some embodiments, R¹⁶ is a cholesterol moiety, substituted with C(O)OR^{L}, C(O)NR^{L}R^{L′}, R^{L}, S(O)ₘR^{L}, or S(O)ₘNR^{L}R^{L′}. In some embodiments, R¹⁶ is a cholesterol moiety, substituted with C(O)NR^{L}R^{L′}. In some embodiments, R^{L} is alkenyl and R^{L′} is H. In some embodiments, R^{L} has a Z configuration. In some embodiments, R^{L} is C¹⁸ alkenyl.

In some embodiments, R¹⁴ is H.

In some embodiments, R¹⁵ is alkyl optionally substituted with 1-3 nitrogen containing moieties selected from the group consisting of NR¹⁸R¹⁹ or a nitrogen containing heterocyclyl.

In some embodiments, R¹⁵ is alkyl optionally substituted with 1-3 nitrogen containing moieties selected from the group consisting of NR¹⁸R¹⁹ or a nitrogen containing heterocyclyl and R¹⁴ is H.

In some embodiments, R¹⁵ is C₁-C₆ alkyl substituted with a nitrogen containing moiety selected from the group consisting of NR¹⁸R¹⁹ or a nitrogen containing heterocyclyl.

In some embodiments, R¹⁵ is C₁-C₆ alkyl substituted with a nitrogen containing moiety selected from the group consisting of NR¹⁸R¹⁹ or a nitrogen containing heterocyclyl and R¹⁴ is H.

In some embodiments, R¹⁵ is C₁-C₆ alkyl substituted NR¹⁸R¹⁹. In some embodiments, R¹⁸ and R¹⁹ are both alkyl. In some embodiments, R¹⁸ and R¹⁹ are both C₁-C₆ alkyl. In some embodiments, R¹⁸ and R¹⁹ are both methyl.

In some embodiments, wherein R¹⁵ is

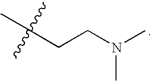

In some embodiments, R¹⁴ is H.

In some embodiments, R¹⁵ is C₁-C₆ alkyl substituted with a nitrogen containing heterocyclyl. In some embodiments, the nitrogen containing heterocyclyl has 2 ring nitrogens. In some embodiments, the nitrogen containing heteroaryl has 2 ring nitrogens. In some embodiments, heteroaryl is an imidazolyl. In some embodiments, R¹⁵ is

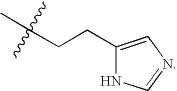

In some embodiments, both R¹⁴ and R¹⁵ are C₁-C₆ alkyl substituted NR¹⁸R¹⁹. In some embodiments, both R¹⁴ and R¹⁵ are

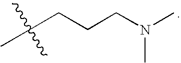

In some embodiments, one or both of R¹⁴ and R¹⁵ are alkyl. In some embodiments, one or both of R¹⁴ and R¹⁵ is C₁₀₋₃₀ alkyl. In some embodiments, one or both of R¹⁴ and R¹⁵ is C₁₀₋₁₈ alkyl. In some embodiments, one or both of R¹⁴ and R¹⁵ is C₁₂ alkyl.

In some embodiments, one or both of R¹⁴ and R¹⁵ is alkenyl. In some embodiments, one or both of R¹⁴ and R¹⁵ is C₆-C₃₀ alkenyl. In some embodiments, one or both of R¹⁴ and R¹⁵ has a single double bond. In some embodiments, the double bond has a Z configuration. In some embodiments, one or both of R¹⁴ and R¹⁵ has two double bonds. In some embodiments, at least one of the double bonds have a Z configuration. In some embodiments, both of the double bonds have a Z configuration. In some embodiments, one or both of R¹⁴ and R¹⁵ has the following formula:

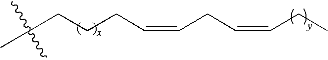

wherein

x is an integer from 1 to 8; and

y is an integer from 1-10. one or both of R¹⁴ and R¹⁵ is

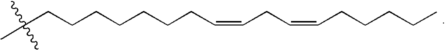

In some embodiments, at least one of the double bonds has an E configuration. In some embodiments, both of the double bonds have an E configuration. In some embodiments, one or both of R¹⁴ and R¹⁵ has the following formula:

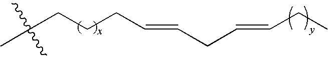

wherein

x is an integer from 1 to 8; and

y is an integer from 1-10. In some embodiments, one or both of R¹⁴ and R¹⁵ has three double bond moieties. In some embodiments, at least one of the double bonds has a Z configuration. In some embodiments, at least two of the double bonds have a Z configuration. In some embodiments, all three of the double bonds have a Z configuration. In some embodiments, one or both of R¹⁴ and R¹⁵ has the following formula:

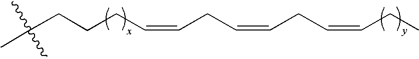

wherein

x is an integer from 1 to 8; and

y is an integer from 1-10. In some embodiments, at least one of the double bonds have an E configuration. In some embodiments, at least two of the double bonds have an E configuration. In some embodiments, all three of the double bonds have an E configuration. In some embodiments, one or both of R¹⁴ and R¹⁵ has the following formula:

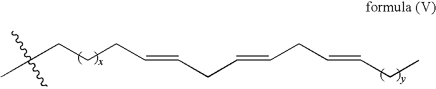

wherein

x is an integer from 1 to 8; and

y is an integer from 1-10.

In some embodiments, one or both of R¹⁴ and R¹⁵ is alkynyl.

A method of a making a cyclic lipid of formula (III), the method comprising reacting a compound of formula (VI)

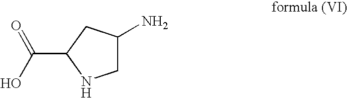

by alkylating or amidating the exocyclic amine with a lipophilic moiety; and

optionally coupling a lipophilic moiety or cationic moiety with the carboxylic acid and/or reacting a cationic moiety to the ring nitrogen thereby making a cyclic lipid.

In some embodiments, a compound described herein such as a compound of formula (I), (II), (III), or (IV) represents a diastereomeric mixture (e.g. a preparation of a diastereomeric compound).

In some embodiments, the compound of formula (I), (II), (III), or (IV) has an diastereomeric excess of a single isomer, e.g., at least about 65%, 70%, 75%, 80%, 85%, 90%, 95%, 97%, 98%, or 99%.

In some embodiments, the compound is enriched for a single diastereomer, for example, the compound of formula (III) or (IV) is enriched for an R,R isomer, an R,S isomer, and S,R isomer or an S,S, isomer. For example, the compound can be enriched to have at least about 65%, 70%, 75%, 80%, 85%, 90%, 95%, 97%, 98%, or 99% of the recited diastereomer or be a substantially pure compound of one of the following diastereomers: an R,R isomer, an R,S isomer, and S,R isomer or an S,S, isomer.

In some embodiments, a cyclic lipid described herein is separated from a reaction mixture using chromatographic separation. In some embodiments, the chromatographic separation is using flash silica gel for separation of isomers. In some embodiments, the chromatographic separation is gravity separation of isomers using silica gel. In some embodiments, the chromatographic separation is using moving bed chromatography for separation of isomers. In some embodiments, the chromatographic separation uses liquid chromatography (LC) for separation of isomers. In some embodiments, the chromatographic separation is normal phase HPLC for separation of isomers. In some embodiments, the chromatographic separation is reverse phase HPLC for separation of isomers.

In one aspect, the invention features a preparation including a cyclic lipid described herein, for example a compound of formula (I), (II), (III), or (IV).

In one aspect, the invention features a preparation including a cyclic lipid described herein, for example a compound of formula (I), (II), (III), or (IV) and a nucleic acid (e.g., an RNA such as an siRNA or dsRNA or a DNA). In some embodiments, the preparation includes one or more additional lipids such as a fusogenic lipid, or a PEG-lipid. In some embodiments, the preparation includes a targeting moiety.

In one aspect, the invention features an association complex, such as a liposome, comprising a preparation described herein (e.g., a lipid preparation comprising a compound of formula (I), (II), (III), or (IV)) and a nucleic acid. In some embodiments, the preparation also includes a PEGylated lipid, for example a PEG-lipid described herein. In some embodiments, the preparation also includes a structural moiety such as cholesterol. In some embodiments the preparation of the association complex includes compounds of formulae (I), (II), (III), or (IV) and cholesterol. In some embodiments, said nucleic acid is an siRNA, for example said nucleic acid is an siRNA which has been modified to resist degradation, said nucleic acid is an siRNA which has been modified by modification of the polysaccharide backbone, or said siRNA targets the ApoB gene.

In some embodiments, the liposome further comprises a structural moiety and a PEGylated lipid, such as a PEG-lipid described herein, wherein the ratio, by weight cyclic lipid such as a compound of formula (I), (II), (III), or (IV), structural moiety, PEGylated lipid, and a nucleic acid, is 8-22:4-10:4-12:0.4-2.2. In some embodiments, the structural moiety is cholesterol. In some embodiments, the ratio is 10-20:0.5-8.0:5-10:0.5-2.0, e.g., 15:0.8:7:1. In some embodiments, the average liposome diameter is between 10 nm and 750 nm, e.g., the average liposome diameter is between 30 and 200 nm or the average liposome diameter is between 50 and 100 nm. In some embodiments, the preparation is less than 15%, by weight, of unreacted lipid.

In some embodiments an association complex described herein has a weight ratio of total excipients to nucleic acid of less than about 20:1, for example, about, 15:1 10:1, 7.5:1 or about 5:1.

In one aspect, the invention features a method of forming an association complex comprising a plurality of lipid moieties and a therapeutic agent, the method comprising: mixing a plurality of lipid moieties in a solvent and buffer such as ethanol and aqueous NaOAc buffer, to provide a particle; and adding the therapeutic agent to the particle, thereby forming the association complex.

In some embodiments, the lipid moieties are provided in a solution of 100% ethanol.

In some embodiments, the plurality of lipid moieties comprise a cyclic lipid.

### DEFINITIONS

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₃₆ alkyl indicates that the group may have from 1 to 36 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, —CH₂CH₂CH₂—, —CH₂CH₂CH₂CH₂⁻, —CH₂CH₂CH₂CH₂CH₂—, and CH₂CH₂CH₂CH₂CH₂CH₂—.

The term “alkenyl” refers to a straight or branched hydrocarbon chain containing 2-36 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-36 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to same carbon (geminal substitution) atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

The term “cationic group” means that group carries a net positive charge at about physiological pH. Examples of cationic groups include, but are not limited to, primary amines, secondary amines, tertiary amines, quartenary amines and the like.

The term “lipophilic group” means that group has a higher affinity for lipids than its affinity for water. Examples of lipophilic groups include, but are not limited to, cholesterol, adamantine, dihydrotesterone, long chain alkyl, long chain alkenyl, long chain alkynyl, olely-lithocholic, cholenic, oleoyl-cholenic, palmityl, heptadecyl, myrisityl and the like.

“Heterocycle” means a 5- to 7-membered monocyclic, or 7- to 10-membered polycyclic, heterocyclic ring which is either saturated, unsaturated, or aromatic, and which contains from 1 or 2 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined below. Heterocycles include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heteroaryl” means a monocyclic- or polycyclic aromatic ring comprising carbon atoms, hydrogen atoms, and one or more heteroatoms, preferably, 1 to 3 heteroatoms, independently selected from nitrogen, oxygen, and sulfur. As is well known to those skilled in the al, heteroaryl rings have less aromatic character than their all-carbon counter parts. Thus, for the purposes of the invention, a heteroaryl group need only have some degree of aromatic character. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidinyl, tetrazolyl.

The term “nitrogen protecting group,” as used herein, refers to a labile chemical moiety which is known in the art to protect an amino group against undesired reactions during synthetic procedures. After said synthetic procedure(s) the nitrogen protecting group as described herein may be selectively removed. Nitrogen protecting groups as known in the art are described generally in T. H. Greene and P. G. M. Wuts, Protective Groups in Organic Synthesis, 3rd edition, John Wiley & Sons, New York (1999). Examples of nitrogen protecting groups include, but are not limited to, t-butoxycarbonyl, 9-fluorenylmethoxycarbonyl, benzyloxycarbonyl, and the like.

### Oligopeptides

Oligoeptides suitable for use with the present invention can be a natural peptide, e.g. tat or antennopedia peptide, a synthetic peptide or a peptidomimetic. Furthermore, the peptide can be a modified peptide, for example peptide can comprise non-peptide or pseudo-peptide linkages, and D-amino acids. A peptidomimetic (also referred to herein as an oligopeptidomimetic) is a molecule capable of folding into a defined three-dimensional structure similar to a natural peptide. The attachment of peptide and peptidomimetics to the lipid can affect pharmacokinetic distribution of the lipid particle, such as by enhancing cellular recognition and absorption. The peptide or peptidomimetic moiety can be about 5-50 amino acids long, e.g., about 5, 10, 15, 20, 25, 30, 35, 40, 45, or 50 amino acids long (see Table 3, for example).

TABLE 3
Examplary cell permeation oligopeptides.

| Cell | | |
|---|---|---|
| Permeation | | |
| Peptide | Amino acid Sequence | Reference |
| Penetratin | RQIKIWFQNRRMKWKK | Derossi et al., J. Biol. |
| | | Chem. 269:10444, |
| | | 1994 |
| Tat fragment | GRKKRRQRRRPPQC | Vives et al., J. Biol. |
| (48-60) | | Chem., 272:16010, |
| | | 1997 |
| Signal | GALFLGWLGAAGSTMGAWSQ | Chaloin et al., |
| Sequence- | PKKKRKV | Biochem. Biophys. |
| based peptide | | Res. Commun., |
| | | 243:601, 1998 |
| PVEC | LLIILRRRIRKQAHAHSK | Elmquist et al., Exp. |
| | | Cell Res., 269:237, |
| | | 2001 |
| Transportan | GWTLNSAGYLLKINLKALAAL | Pooga et al., FASEB J., |
| | AKKIL | 12:67, 1998 |
| Amphiphilic | KLALKLALKALKAALKLA | Oehlke et al., Mol. |
| model peptide | | Ther., 2:339, 2000 |
| Arg₉ | RRRRRRRRR | Mitchell et al., J. Pept. |
| | | Res., 56:318, 2000 |
| Bacterial | KFFKFFKFFK | |
| cell wall | | |
| permeating | | |
| LL-37 | LLGDFFRKSKEKIGKEFKRIVQ | |
| | RIKDFLRNLVPRTES | |
| Cecropin P1 | SWLSKTAKKLENSAKKRISEGI | |
| | AIAIQGGPR | |
| α-defensin | ACYCRIPACIAGERRYGTCIYQ | |
| | GRLWAFCC | |
| b-defensin | DHYNCVSSGGQCLYSACPIFTK | |
| | IQGTCYRGKAKCCK | |
| Bactenecin | RKCRIVVIRVCR | |
| PR-39 | RRRPRPPYLPRPRPPPFFPPRLPP | |
| | RIPPGFPPRFPPRFPGKR-NH2 | |
| Indolicidin | ILPWKWPWWPWRR-NH2 | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. A RFGF analogue (e.g., amino acid sequence AALLPVLLAAP) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ) and the Drosophila Antennapedia protein (RQIKIWFQNRRMKWKK) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to the lipid is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide moiety can be used to target a tumor cell, such as an endothelial tumor cell or a breast cancer tumor cell (Zitzmann et al., Cancer Res., 62:5139-43, 2002). An RGD peptide can facilitate targeting to tumors of a variety of other tissues, including the lung, kidney, spleen, or liver (Aoki et al., Cancer Gene Therapy 8:783-787, 2001). Preferably, the RGD peptide will facilitate targeting of the lipid particle to the kidney. The RGD peptide can be linear or cyclic, and can be modified, e.g., glycosylated or methylated to facilitate targeting to specific tissues. For example, a glycosylated RGD peptide can target a tumor cell expressing α_{V}β₃ (Haubner et al., Jour. Nucl. Med., 42:326-336, 2001).

Peptides that target markers enriched in proliferating cells can be used. E.g., RGD containing peptides and peptidomimetics can target cancer cells, in particular cells that exhibit an Iᵥθ₃ integrin. Thus, one could use RGD peptides, cyclic peptides containing RGD, RGD peptides that include D-amino acids, as well as synthetic RGD mimics. In addition to RGD, one can use other moieties that target the Iᵥ-θ₃ integrin ligand. Generally, such ligands can be used to control proliferating cells and angiogeneis.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

The term “structural isomer” as used herein refers to any of two or more chemical compounds, such as propyl alcohol and isopropyl alcohol, having the same molecular formula but different structural formulas.

The term “geometric isomer” or “stereoisomer” as used herein refers to two or more compounds which contain the same number and types of atoms, and bonds (i.e., the connectivity between atoms is the same), but which have different spatial arrangements of the atoms, for example cis and trans isomers of a double bond, enantiomers, and diastereomers.

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A” and “U” each generally stand for a nucleotide that contains guanine, cytosine, adenine, and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

As used herein, “target sequence” refers to a contiguous portion of the nucleotide sequence of an mRNA molecule formed during the transcription of the corresponding gene, including mRNA that is a product of RNA processing of a primary transcription product. A target region is a segment in a target gene that is complementary to a portion of the RNAi agent.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, an oligonucleotide agent comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the sense strand and the antisense strand of an oligonucleotide agent, or between the antisense strand of an oligonucleotide agent and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest. For example, a polynucleotide is complementary to at least a part of an ApoB mRNA if the sequence is substantially complementary to a non-interrupted portion of a mRNA encoding ApoB.

As used herein, an “oligonucleotide agent” refers to a single stranded oligomer or polymer of ribonucleic acid (RNA) or deoxyribonucleic acid (DNA) or both or modifications thereof, which is antisense with respect to its target. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Oligonucleotide agents include both nucleic acid targeting (NAT) oligonucleotide agents and protein-targeting (PT) oligonucleotide agents. NAT and PT oligonucleotide agents refer to single stranded oligomers or polymers of ribonucleic acid (RNA) or deoxyribonucleic acid (DNA) or both or modifications thereof. This term includes oligonucleotides composed of naturally occurring nucleobases, sugars, and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for nucleic acid target, and/or increased stability in the presence of nucleases. NATs designed to bind to specific RNA or DNA targets have substantial complementarity, e.g., at least 70, 80, 90, or 100% complementary, with at least 10, 20, or 30 or more bases of a target nucleic acid, and include antisense RNAs, microRNAs, antagomirs and other non-duplex structures which can modulate expression. Other NAT oligonucleotide agents include external guide sequence (EGS) oligonucleotides (oligozymes), DNAzymes, and ribozymes. The NAT oligonucleotide agents can target any nucleic acid, e.g., a miRNA, a pre-miRNA, a pre-mRNA, an mRNA, or a DNA. These NAT oligonucleotide agents may or may not bind via Watson-Crick complementarity to their targets. PT oligonucleotide agents bind to protein targets, preferably by virtue of three-dimensional interactions, and modulate protein activity. They include decoy RNAs, aptamers, and the like.

While not wishing to be bound by theory, an oligonucleotide agent may act by one or more of a number of mechanisms, including a cleavage-dependent or cleavage-independent mechanism. A cleavage-based mechanism can be RNAse H dependent and/or can include RISC complex function. Cleavage-independent mechanisms include occupancy-based translational arrest, such as can be mediated by miRNAs, or binding of the oligonucleotide agent to a protein, as do aptamers. Oligonucleotide agents may also be used to alter the expression of genes by changing the choice of splice site in a pre-mRNA. Inhibition of splicing can also result in degradation of the improperly processed message, thus down-regulating gene expression.

The term “double-stranded RNA” or “dsRNA”, as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where separate RNA molecules, such dsRNA are often referred to in the literature as siRNA (“short interfering RNA”). Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”, “short hairpin RNA” or “shRNA”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs. In addition, as used in this specification, “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of this specification and claims.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule. For clarity, chemical caps or non-nucleotide chemical moieties conjugated to the 3′ end or 5′ end of an siRNA are not considered in determining whether an siRNA has an overhang or is blunt ended.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

The terms “silence” and “inhibit the expression of”, in as far as they refer to a target gene, herein refer to the at least partial suppression of the expression of the gene, as manifested by a reduction of the amount of mRNA transcribed from the gene which may be isolated from a first cell or group of cells in which the gene is transcribed and which has or have been treated such that the expression of the gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to gene transcription, e.g. the amount of protein encoded by the gene which is secreted by a cell, or the number of cells displaying a certain phenotype, e.g apoptosis. In principle, gene silencing may be determined in any cell expressing the target, either constitutively or by genomic engineering, and by any appropriate assay. However, when a reference is needed in order to determine whether a given dsRNA inhibits the expression of the gene by a certain degree and therefore is encompassed by the instant invention, the assay provided in the Examples below shall serve as such reference.

For example, in certain instances, expression of the gene is suppressed by at least about 20%, 25%, 35%, or 50% by administration of the double-stranded oligonucleotide of the invention. In some embodiment, the gene is suppressed by at least about 60%, 70%, or 80% by administration of the double-stranded oligonucleotide of the invention. In some embodiments, the gene is suppressed by at least about 85%, 90%, or 95% by administration of the double-stranded oligonucleotide of the invention.

As used herein, the terms “treat”, “treatment”, and the like, refer to relief from or alleviation of pathological processes which can be mediated by down regulating a particular gene. In the context of the present invention insofar as it relates to any of the other conditions recited herein below (other than pathological processes which can be mediated by down regulating the gene), the terms “treat”, “treatment”, and the like mean to relieve or alleviate at least one symptom associated with such condition, or to slow or reverse the progression of such condition.

As used herein, the phrases “therapeutically effective amount” and “prophylactically effective amount” refer to an amount that provides a therapeutic benefit in the treatment, prevention, or management of pathological processes which can be mediated by down regulating the gene on or an overt symptom of pathological processes which can be mediated by down regulating the gene. The specific amount that is therapeutically effective can be readily determined by ordinary medical practitioner, and may vary depending on factors known in the art, such as, e.g. the type of pathological processes which can be mediated by down regulating the gene, the patient's history and age, the stage of pathological processes which can be mediated by down regulating gene expression, and the administration of other anti-pathological processes which can be mediated by down regulating gene expression. An effective amount, in the context of treating a subject, is sufficient to produce a therapeutic benefit. The term “therapeutic benefit” as used herein refers to anything that promotes or enhances the well-being of the subject with respect to the medical treatment of the subject's cell proliferative disease. A list of nonexhaustive examples of this includes extension of the patients life by any period of time; decrease or delay in the neoplastic development of the disease; decrease in hyperproliferation; reduction in tumor growth; delay of metastases; reduction in the proliferation rate of a cancer cell, tumor cell, or any other hyperproliferative cell; induction of apoptosis in any treated cell or in any cell affected by a treated cell; and/or a decrease in pain to the subject that can be attributed to the patient's condition.

As used herein, a “pharmaceutical composition” comprises a pharmacologically effective amount of an oligonucleotide agent and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof and are described in more detail below. The term specifically excludes cell culture medium.

The details of one or more embodiments of the invention are set forth in the accompanying drawings and the description below. Other features, objects, and advantages of the invention will be apparent from the description and drawings, and from the claims.

### DETAILED DESCRIPTION

Lipid compounds, preparations, and delivery systems useful to administer nucleic acid based therapies such as siRNA are described herein.

### Lipid Compounds and Lipid Preparations

Applicants have discovered that certain lipid moieties (e.g., a cationic lipid such as an amine containing lipid moiety) provide desirable properties for administration of nucleic acids, such as siRNA. Accordingly, lipids providing enhanced in vivo delivery of a nucleic acid such as siRNA are preferred. In particular, Applicants have discovered cyclic amines linked to one or more lipids, for example having substitutions described herein, can have desirable properties for delivering siRNA, such as bioavailability, biodegradability, and tolerability, for example as a component in an association complex, such as a liposome.

The lipid moieties described herein generally include a cyclic moiety, such as a cyclic amine, to which at least one lipid is attached. In some embodiments, two or more lipids are attached to the cyclic moiety, for example, two, three or more lipids are attached to the cyclic moiety. Exemplary cyclic moieties include those provided in formulas (I), (II), (III), and (IV) below, wherein the R moieties defined as herein above.

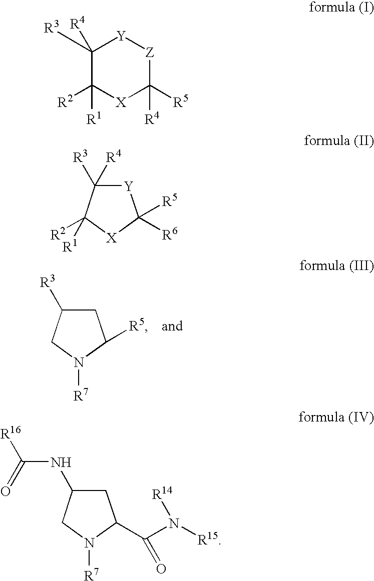

In preferred embodiments, the cyclic moiety is a nitrogen containing moiety such as a five or six membered ring containing one or two nitrogens. Exemplary cyclic moieties include piperazine, piperidine, and pyrrolidine. In some embodiments pyrrolidine is the preferred cyclic moiety. A lipid moiety can be bound to the cyclic moiety through any ring atom, including a ring carbon or a ring nitrogen. In some embodiments, the lipid moiety is bound to the cyclic moiety through a linking atom or group.

In some embodiments the cyclic moiety is substituted with a nitrogen containing moiety, for example, in addition to being substituted with a lipid moiety. The nitrogen containing moiety can, in some instances, provide a cationic portion of the cyclic lipid moiety. In some embodiments, the nitrogen containing moiety includes an amine nitrogen or a nitrogen containing heterocycle such as imidazole. In some embodiments the amine nitrogen is substituted, for example with an alkyl moiety or a BOC group. In some embodiments the nitrogen is unsubstituted.

In some embodiments the cyclic moiety is covalently bound to a single lipid moiety. In instances where the cyclic moiety is bound to a single lipid moiety, the cyclic moiety can be further bound to a second moiety, such as a moiety having a nitrogen containing group. Exemplary nitrogen containing groups include amine nitrogens (including unsubstituted amines and substituted amines e.g., substituted with alkyl or a BOC group) or a nitrogen containing heterocyclic moiety such as an imidazole moiety.

In embodiments the cyclic moiety is covalently bound to two lipid moieties. For example, two lipid moieties can be covalently bound on two carbon atoms of the ring. In some embodiments two lipid moieties are bond to a ring carbon through a nitrogen or other linking atom or group. For example a ring carbon can be substituted by a nitrogen or nitrogen containing group such as an amide, which is further substituted by one or two lipid moieties such as an alkyl, alenyl, alkynyl or cholesterol moiety. In some embodiments two lipid moieties can be bound to a single ring atom such as a ring carbon. For example, two lipid moieties can be attached to a single ring atom through a nitrogen atom or nitrogen containing group bound to the cyclic moiety.

In instances where the cyclic moiety is substituted with two moieties (e.g., a lipid moiety, a nitrogen moiety, or any combinations thereof) the relative stereochemistry of the two can be moieties are cis or trans. In some preferred embodiments, the relative configuration of the two lipid moieties is cis. In some embodiments, the cyclic moiety is present as a mixture of cis and trans configured compounds.

In some embodiments, a cyclic moiety (e.g., a nitrogen containing cyclic moiety) bearing two substituents (i.e., moieties) on two carbon atoms of the ring moiety also includes a substituent on the nitrogen moiety. In some embodiments, the nitrogen atom is unsubstituted. Preferred substituents on a ring nitrogen include BOC and nitrogen containing moieties such as amines (including unsubstituted amines and substituted amines e.g., substituted with alkyl or a BOC group) or a nitrogen containing heterocyclic moiety such as an imidazole moiety.

Where the cyclic moiety has one or more stereocenters, in some embodiments, the resulting lipid moiety has an diastereomeric excess of a preferred isomer, e.g., at least about 65%, 70%, 75%, 80%, 85%, 90%, 95%, 97%, 98%, or 99%. In some embodiments the lipid moiety represents enantiomerically pure isomer. For example, when the cyclic moiety has formula (III) or formula (IV), the lipid moiety can have one of the following configurations: 2R,4R; 2S,4R; 2S,4S and 2R,4S, any of which can be present in an enantiomeric excess.

Exemplary lipid moieties include alkyl, alkenyl, alkynyl moieties and cholesterol (e.g., optionally substituted cholesterol such as cholesterol substituted with lithocholic acid). Some preferred lipids include C₁₀₋₃₀ alkyl (e.g., C₁₀₋₁₈ alkyl such as C₁₅ alkyl), C6₋₃₀ alkenyl e.g., C₁₂₋₂₀ alkenyl having a single cis double bond,

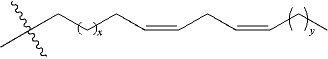

wherein x is an integer from 1 to 8; and y is an integer from 1-10, for example,

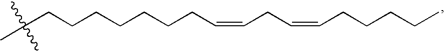

or cholesterol (e.g., unsubstituted or substituted, for example, with lithocholic acid.

In some preferred embodiments, the cyclic lipid has the formula (III) or formula (IV) as provided above.

Where the cyclic lipid is of formula (III) preferred R³ substituents include (CH₂)ₙOR¹³, (CH₂)ₙOC(O)R¹⁶, (CH₂)ₙNR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙ NR¹⁴C(O)R¹⁶, (CH₂)ₙ O—N═CR¹⁶. It is generally preferred that n is 0. In some most preferred embodiments R³ is NR¹⁴C(O)R¹⁶. In some preferred embodiments the R³ substituent includes a lipid moiety as defined above, for example as provided in the defined R groups.

Where the cyclic lipid is of formula (III) preferred R⁵ substituents include (CH₂)ₙOR¹³, (CH₂)ₙC(O)OR¹³, (CH₂)ₙOC(O)R¹⁶, (CH₂)ₙC(O)NR¹⁴R¹⁵. It is generally preferred that n is 0 or 1. In some most preferred embodiments, R⁵ is C(O)NR¹⁴R¹⁵. In some embodiments one of R¹⁴ or R¹⁵ is a lipid moiety as described above (e.g., alkyl, alkenyl, alkynyl moieties and cholesterol including optionally substituted cholesterol such as cholesterol substituted with lithocholic acid. In some embodiments both R¹⁴ and R¹⁵ are a lipid moiety as described above. In some preferred embodiments one of R¹⁴ or R¹⁵ is hydrogen. In some embodiments, one or R¹⁴ or R¹⁵ is an alkyl moiety (e.g., C₁₋₆alkyl substituted with 1-3 nitrogen containing moieties selected from the group consisting of NR¹⁸R¹⁹ (e.g., an amine nitrogen such as an alkyl amine or an amine substituted with BOC) or a nitrogen containing heterocycle such as imidazole.

Where the cyclic lipid is of formula (III) preferred R⁷ substituents include hydrogen, BOC, and C(O)R¹⁶. In some preferred embodiments where R7 is C(O)R¹⁶, R¹⁶ is C₁₋₆alkyl substituted with 1-3 nitrogen containing moieties selected from the group consisting of NR¹⁸R¹⁹ (e.g., an amine nitrogen such as an alkyl amine or an amine substituted with BOC) or a nitrogen containing heterocycle such as imidazole.

In some embodiments, the cyclic lipid described herein is in the form of a salt, such as a pharmaceutically acceptable salt. A salt, for example, can be formed between an anion and a positively charged substituent (e.g., amino) on a compound described herein. Suitable anions include fluoride, chloride, bromide, iodide, sulfate, bisulfate, nitrate, phosphate, citrate, methanesulfonate, trifluoroacetate, acetate, fumarate, oleate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, ethanesulfonate, benzenesulfonate, p-toluensulfonate, and pamoate. In some preferred embodiments, the cyclic lipid is a hydrohalide salt, such as a hydrochloride salt.

Cyclic lipids can also be present in the form of hydrates (e.g., (H₂O)ₙ) and solvates, which are included herewith in the disclosure.

Exemplary cyclic lipids are described below in formulas 1 and 2.

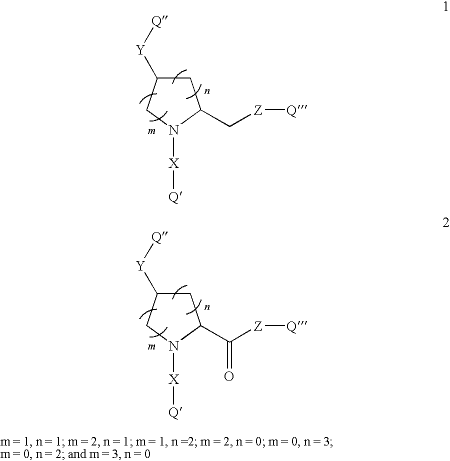

Exemplary lipids include biodegradable, cationic lipids as provided above. The compounds can have racemic and/or stereospecific configurations at each chiral center (see Tables 1 and 2 for examples).

Q′=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety

Q″=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

Q′″=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety (see Tables 1 and 2 for typical examples).

X=—C(O)—; —C(O)—NH—; —C(O)—O—; —(CH₂)—;

Y=NHC(O)—; N(R)C(O)—; —NHC(O)—O—; —N(R)C(O)—O—; —NHC(O)—NH—; —N(R)C(O)—N(R)—; —OC(O)—; —C(O)O—; —OC(O)—NH—; —OC(O)—N(R)—; —C(O)—N(R)—; —C(O)NH—; —S—S—; where, R is Q″

Z=NH—; N(R)—; —O—; —S—; —(CH₂)—; —OC(O)—; —C(O)O—; —OC(O)—NH—; —OC(O)—N(R)—; —C(O)—N(R)—; —C(O)NH—; —S—S—; where, R is Q.

Exemplary cyclic lipid moieties also include those provided in formulas 3 and 4 below:

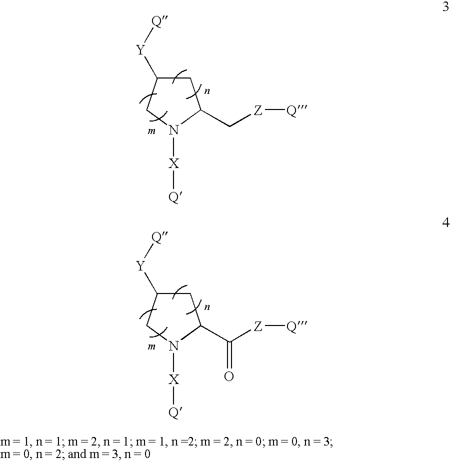

Exemplary lipids include biodegradable cationic lipids with racemic and/or stereopecific configurations at each chiral center (see Tables 1 and 2 for examples).

Q′=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety (see Tables 1 and 2 for typical examples).

Q″=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety

Q′″=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

X=—C(O)—; —C(O)—NH—; —C(O)—O—; —(CH₂)—;

Y=NHC(O)—; N(R)C(O)—; —NHC(O)—O—; —N(R)C(O)—O—; —NHC(O)—NH—; —N(R)C(O)—N(R)—; —OC(O)—; —C(O)O—; —OC(O)—NH—; —OC(O)—N(R)—; —C(O)—N(R)—; —C(O)NH—; —S—S—; where, R is Q″

Z=NH—; N(R)—; —O—; —S—; —(CH₂)—; —OC(O)—; —C(O)O—; —OC(O)—NH—; —OC(O)—N(R)—; —C(O)—N(R)—; —C(O)NH—; —S—S—; where, R is Q″.

Exemplary cyclic lipid moieties also include those provided in formulas 5 and 6 below:

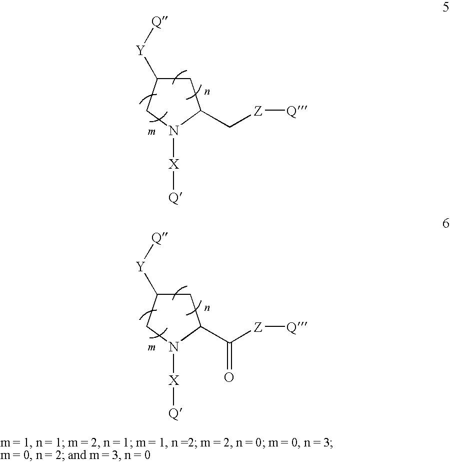

Exemplary lipids include biodegradable cationic lipids with racemic and/or stereopecific configurations at each chiral center (see Tables 1 and 2 for examples).

Q′=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

Q″=A cationic moiety with one or more protanatable nitrogens or protonatable heterocylcles containing nitrogen atoms or combination there of, single D or L amino acid, D or L di, tri, tetra or penta peptide, or combination of D and L di, tri, tetra and penta peptide; or an oligopeptide; or a PEG moiety (see Tables 1 and 2 for typical examples).

Q′″=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety

X=—C(O)—; —C(O)—NH—; —C(O)—O—; —(CH₂)—;

Y=NHC(O)—; N(R)C(O)—; —NHC(O)—O—; —N(R)C(O)—O—; —NHC(O)—NH—; —N(R)C(O)—N(R)—; —OC(O)—; —C(O)O—; —OC(O)—NH—; —OC(O)—N(R)—; —C(O)—N(R)—; —C(O)NH—; —S—S—; where, R is Q″

Z=NH—; N(R)—; —O—; —S—; —(CH₂)—; —OC(O)—; —C(O)O—; —OC(O)—NH—; —OC(O)—N(R)—; —C(O)—N(R)—; —C(O)NH—; —S—S—; where, R is Q″.

Exemplary cyclic lipid moieties also include those provided in formulas 7 and 8 below:

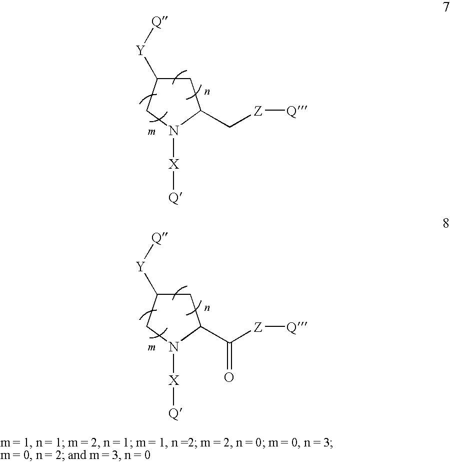

Exemplary cyclic lipids include biodegradable, cationic lipids with racemic and/or stereopecific configurations at each chiral center (see Tables 1 and 2 for examples).

Q′=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

Q″=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

Q′″=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety (see Tables 1 and 2 for typical examples).

X=—C(O)—; —C(O)—NH—; —C(O)—O—; —(CH₂)—;

Y=NHC(O)—; N(R)C(O)—; —NHC(O)—O—; —N(R)C(O)—O—; —NHC(O)—NH—; —N(R)C(O)—N(R)—; —OC(O)—; —C(O)O—; —OC(O)—NH—; —OC(O)—N(R)—; —C(O)—N(R)—; —C(O)NH—; —S—S—; where, R is Q″

Z=NH—; N(R)—; —O—; —S—; —(CH₂)—; —OC(O)—; —C(O)O—; —OC(O)—NH—; —OC(O)—N(R)—; —C(O)—N(R)—; —C(O)NH—; —S—S—; where, R is Q″.

Exemplary cyclic lipid moieties also include those provided in formulas 9 and 10 below:

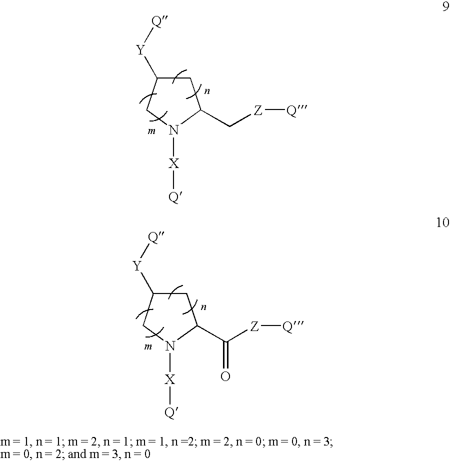

Exemplary cyclic lipids include biodegradable, cationic lipids with racemic and/or stereopecific configurations at each chiral center (see Tables 1 and 2 for examples).

Q′=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety (see Tables 1 and 2 for typical examples).

Q″=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

Q′″=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

X=—C(O)—; —C(O)—NH—; —C(O)—O—; —(CH₂)—;

Y=NHC(O)—; N(R)C(O)—; —NHC(O)—O—; —N(R)C(O)—O—; —NHC(O)—NH—; —N(R)C(O)—N(R)—; —OC(O)—; —C(O)O—; —OC(O)—NH—; —OC(O)—N(R)—; —C(O)—N(R)—; —C(O)NH—; —S—S—; where, R is Q″

Z=NH—; N(R)—; —O—; —S—; —(CH₂)—; —OC(O)—; —C(O)O—; —OC(O)—NH—; —OC(O)—N(R)—; —C(O)—N(R)—; —C(O)NH—; —S—S—; where, R is Q″.

Exemplary cyclic lipid moieties also include those provided in formulas 11 and 12 below:

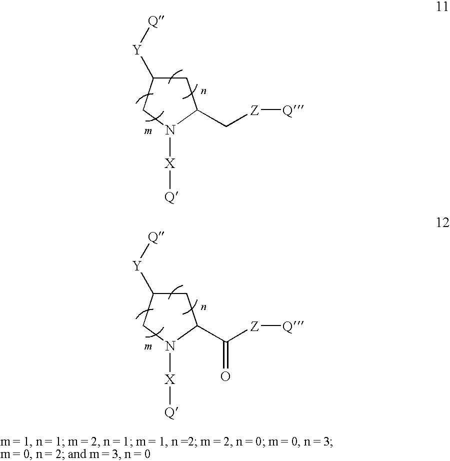

Exemplary cyclic lipids include biodegradable, cationic lipids with racemic and/or stereopecific configurations at each chiral center (see Tables 1 and 2 for examples).

Q′=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

Q″=A cationic moiety with one or more protanatable nitrogens or protonatable heterocylcles containing nitrogen atoms or combination there of; single D or L amino acid, D or L di, tri, tetra or penta peptide, or combination of D and L di, tri, tetra and penta peptide; or an oligopeptide; or a PEG moiety (see Tables 1 and 2 for typical examples).

Q′″=C₆₋₃₂ alkyl; C₆₋₂₃ alkyl with single double bond, for example: oleyl; C₆₋₂₃ alkyl with two double bond, for example: linoleyl; C₆₋₂₃ alkyl with three double bond, for example: eicosatrienyl; C₆₋₂₃ alkyl with one or more triple bonds; cholesteryl; a steroid moiety, a bile acid moiety; 1,2-di-O-alkyl-sn-glyceryl with symmetric and or unsymmetric alkyl chains with chain lengths from C₁₀₋₃₂; 1,2-di-O-alkyl-sn-glyceryl with symmetric and or unsymmetric alkyl chains with chain lengths from C₁₀₋₃₂ having one or more double bonds in one chain or in both chains; 1,2-di-O-acyl-sn-glyceryl with symmetric and or unsymmetric alkyl chains with chain lengths from C₁₀₋₃₂; 1,2-di-O-acyl-sn-glyceryl with symmetric and or unsymmetric alkyl chains with chain lengths from C₁₀₋₃₂ having one or more double bonds in one chain or in both chains.

X=—C(O)—; —C(O)—NH—; —C(O)—O—; —(CH₂)—;

Y=NHC(O)—; N(R)C(O)—; —NHC(O)—O—; —N(R)C(O)—O—; —NHC(O)—NH—; —N(R)C(O)—N(R)—; —OC(O)—; —C(O)O—; —OC(O)—NH—; —OC(O)—N(R)—; —C(O)—N(R)—; —C(O)NH—; —S—S—; where, R is Q″

Z=NH—; N(R)—; —O—; —S—; —(CH₂)—; —OC(O)—; —C(O)O—; —OC(O)—NH—; —OC(O)—N(R)—; —C(O)—N(R)—; —C(O)NH—; —S—S—; where, R is Q″

Exemplary cyclic lipids are provided in Tables 1 and 2 below and are provided in both the form of a free base as well as the corresponding HCl salt. The exemplary lipids provided below have a broad pKₐ distribution. For example, compounds with two or more protonatable nitrogens having pKₐ range between acidic and basic pHs, for example, pKₐ of triethylenetetramine at 20° C. are: 3.32, 6.67, 9.20 and 9.92.

TABLE 1
Examplary cyclic lipids.

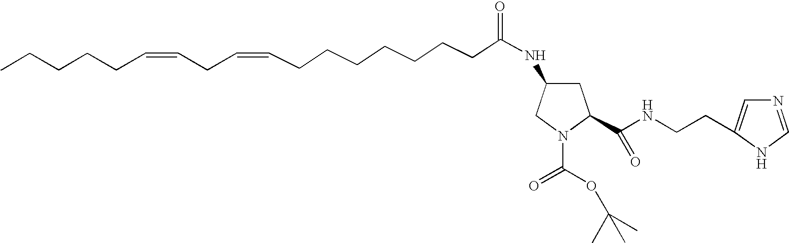

1

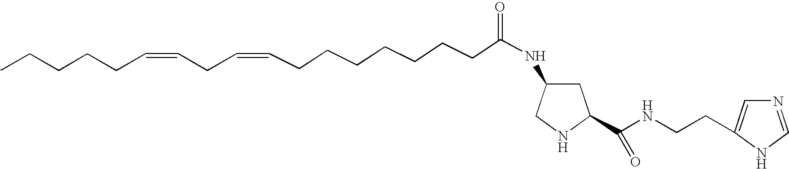

2

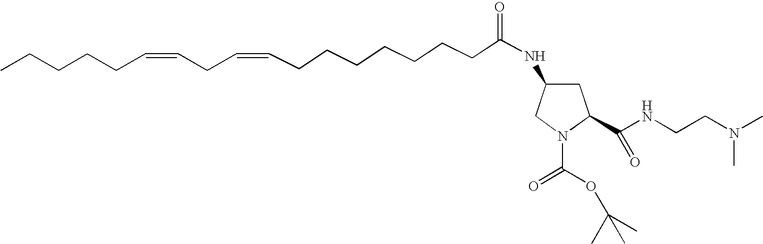

3

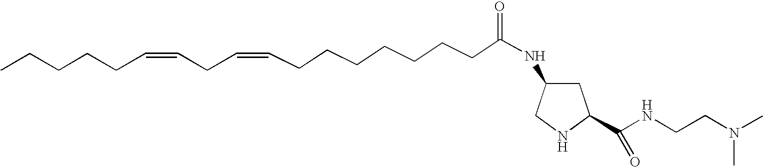

4

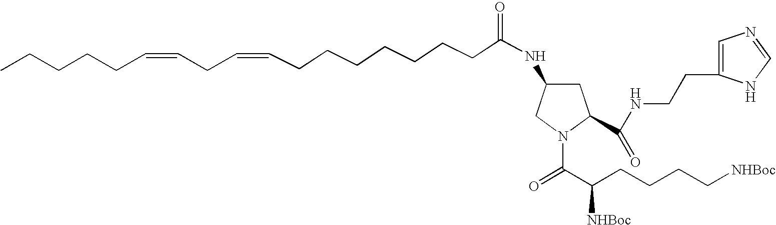

5

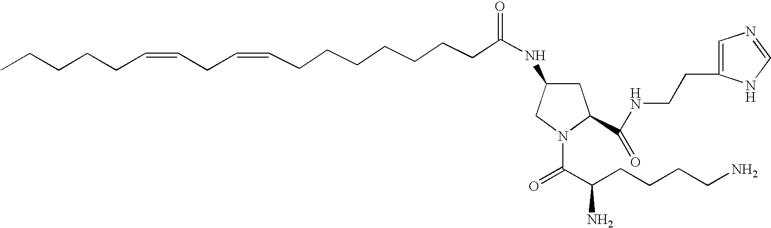

6

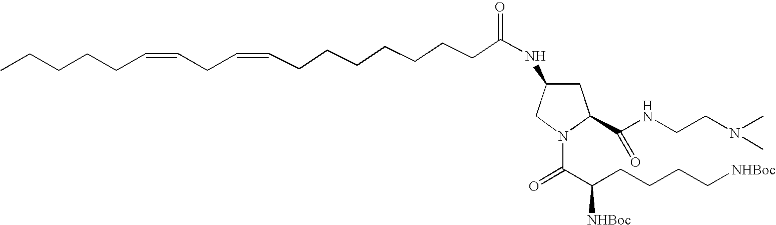

7

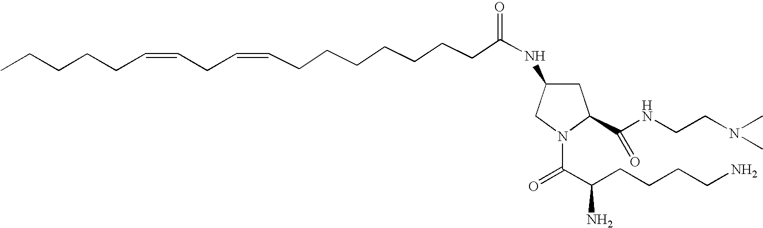

8

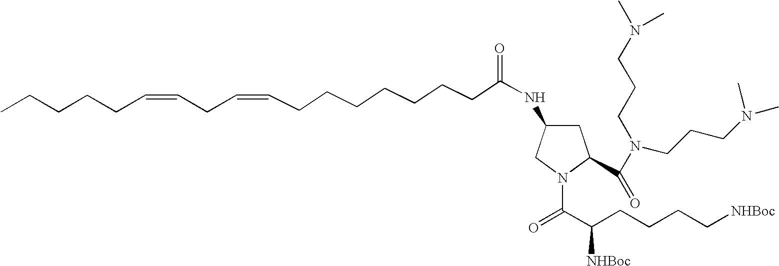

9

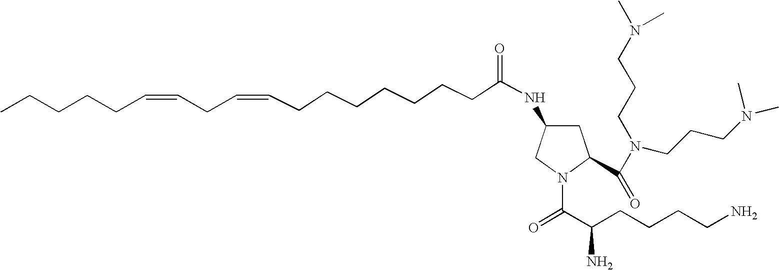

10

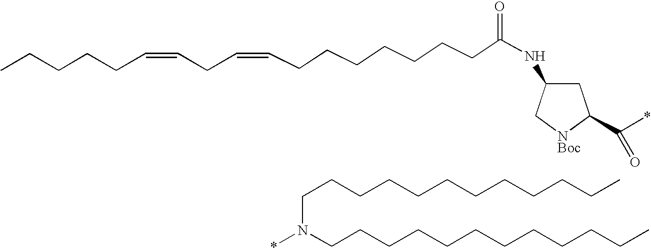

11

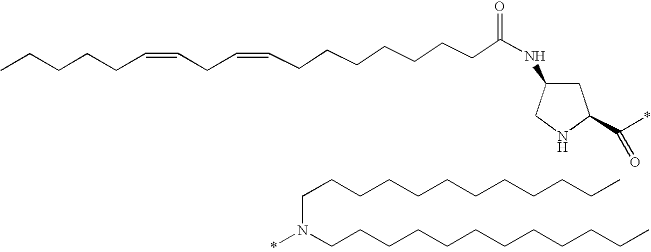

12

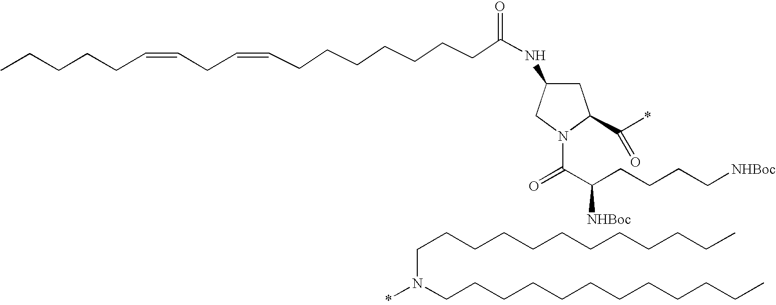

13

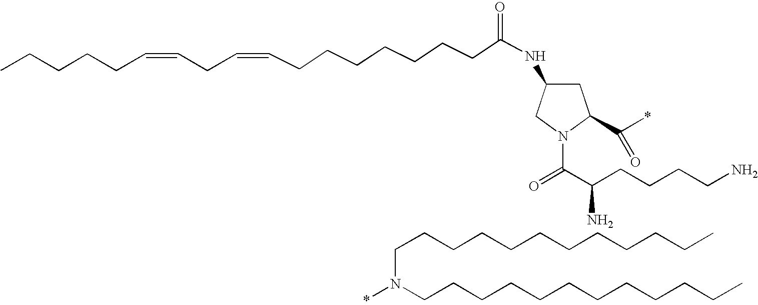

14

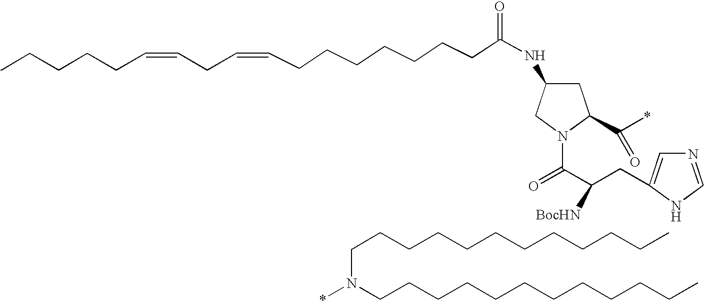

15

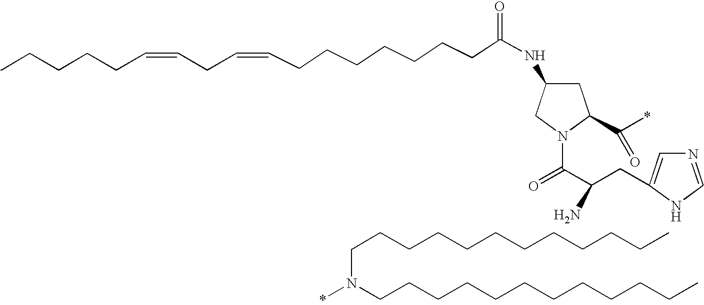

16

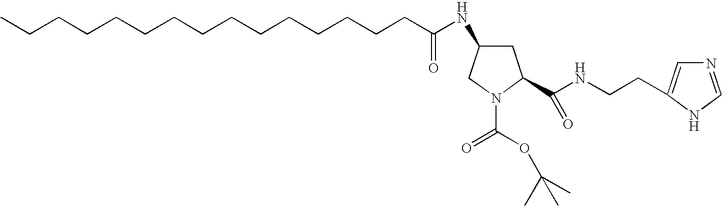

17

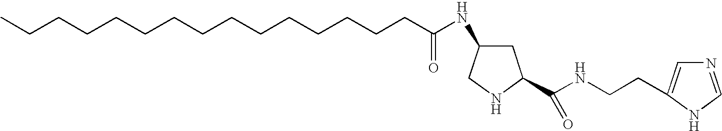

18

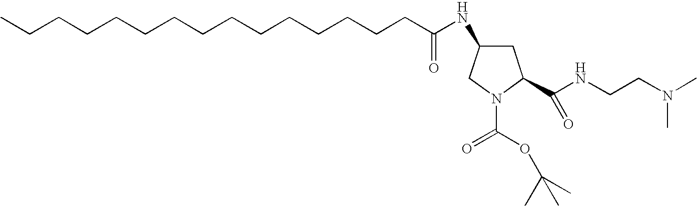

19

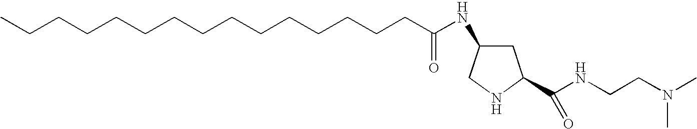

20

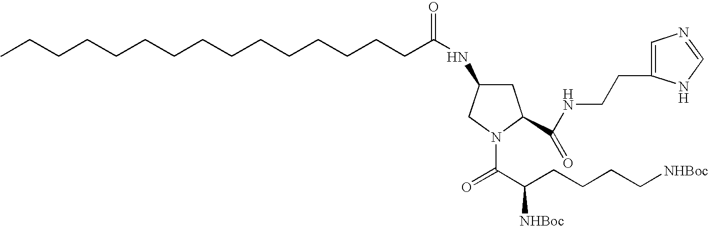

21

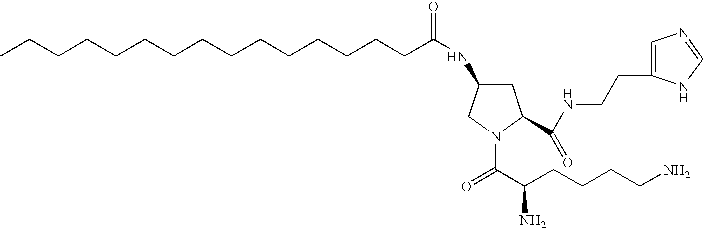

22

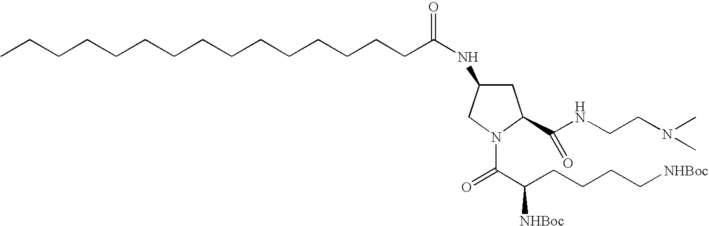

23

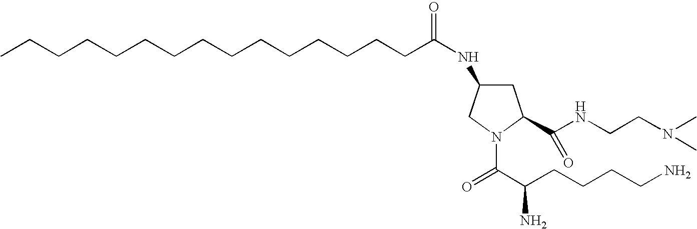

24

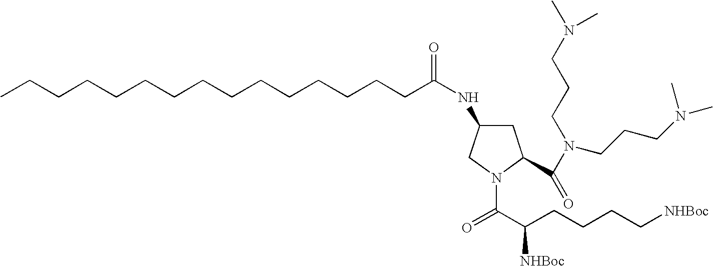

25

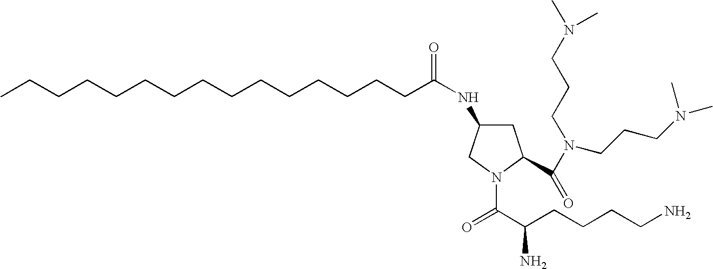

26

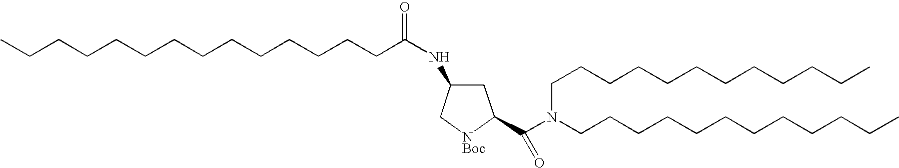

27

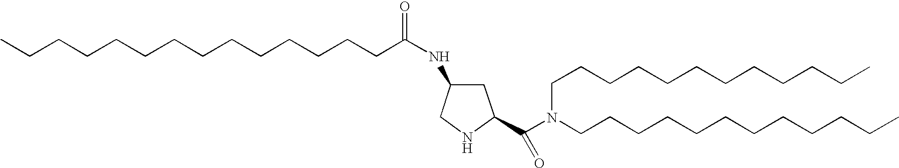

28

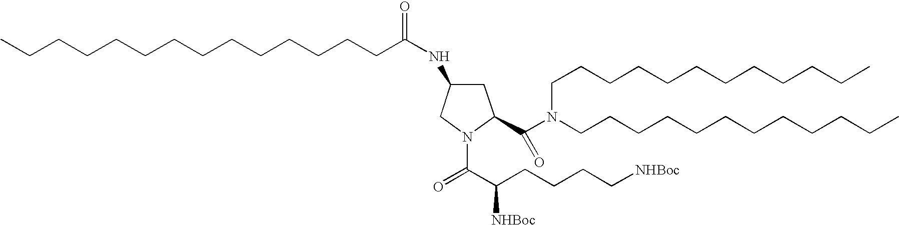

29

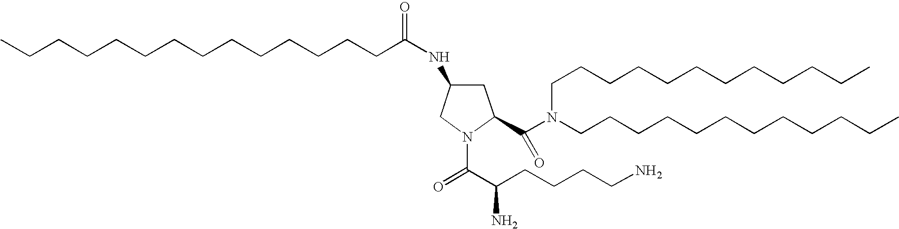

30

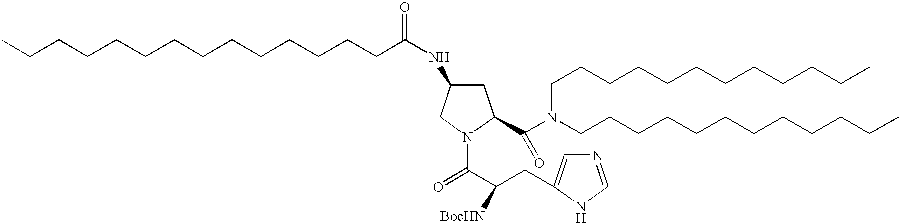

31

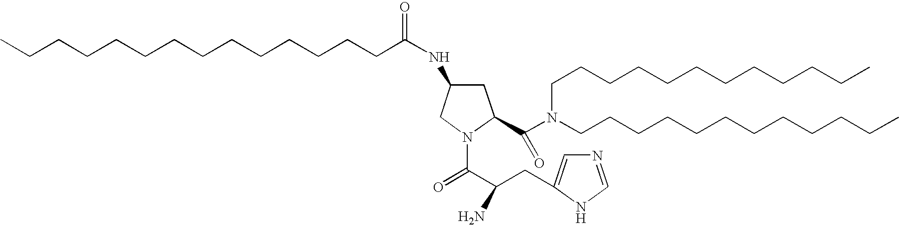

32

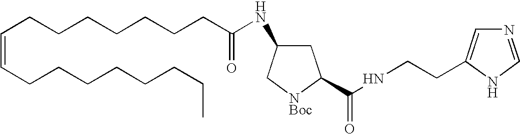

33

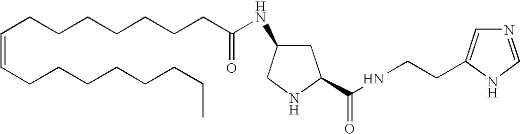

34

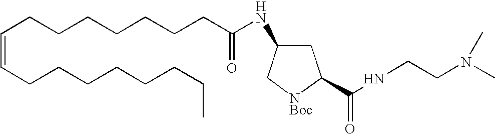

35

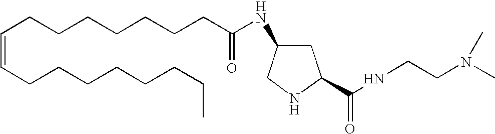

36

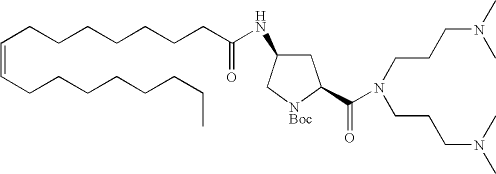

37

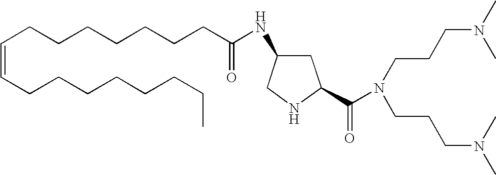

38

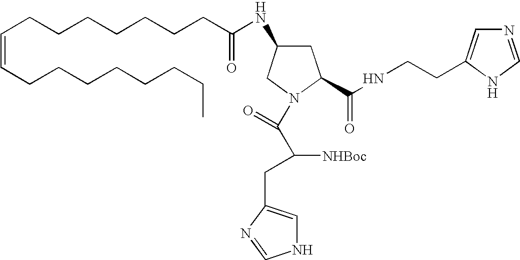

39

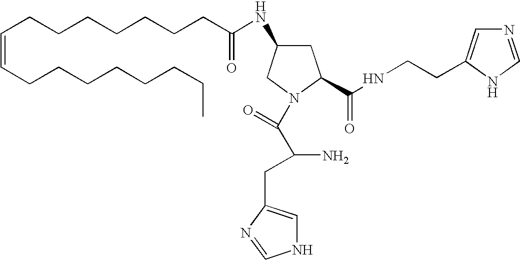

40

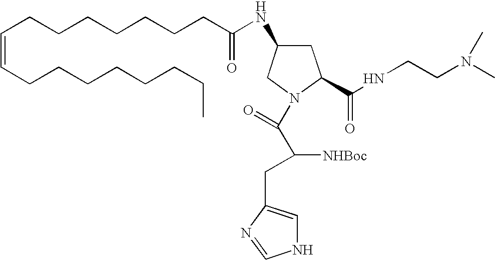

41

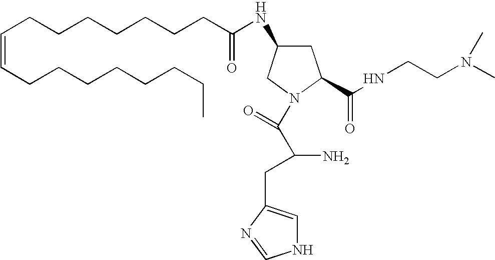

42

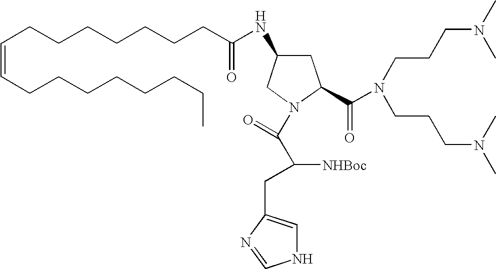

43

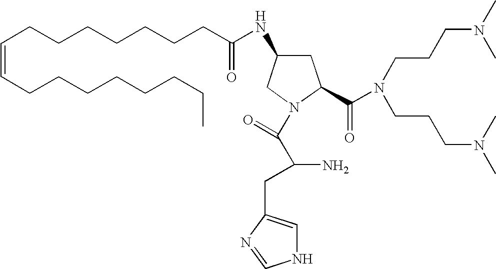

44

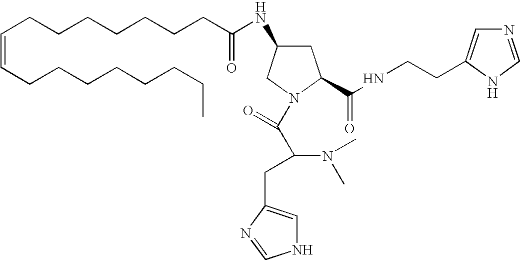

45

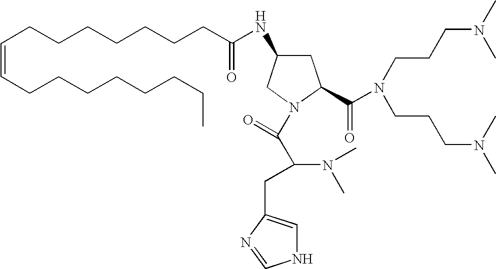

46

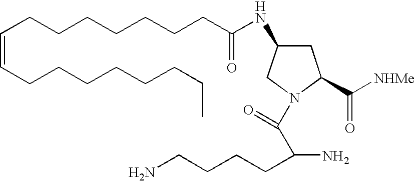

47

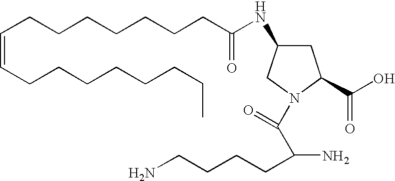

48

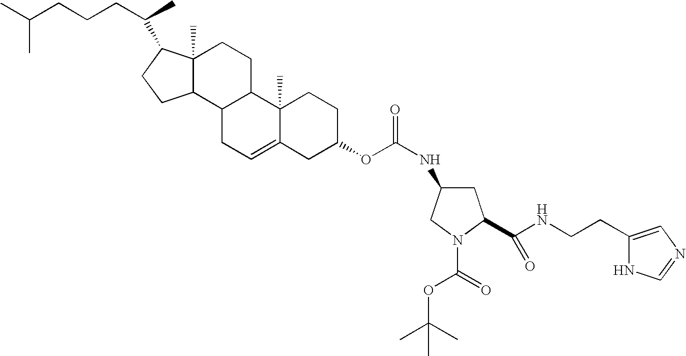

49

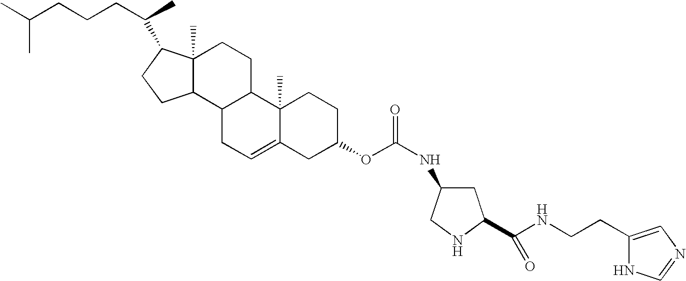

50

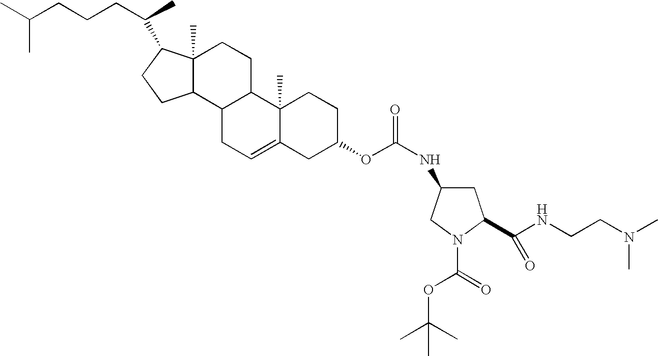

51

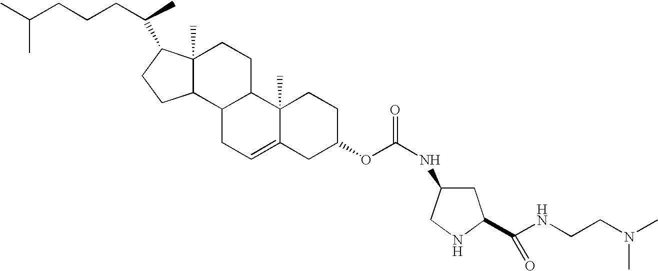

52

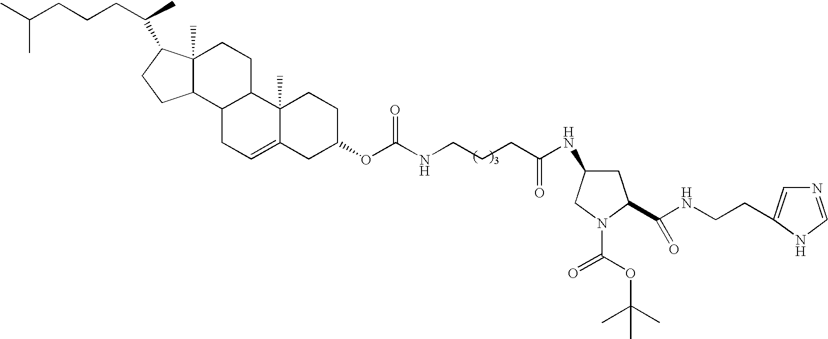

53

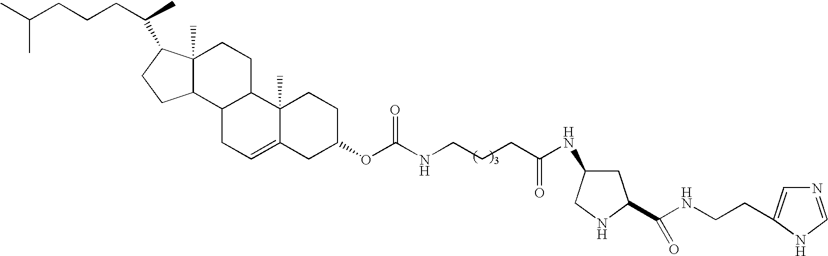

54

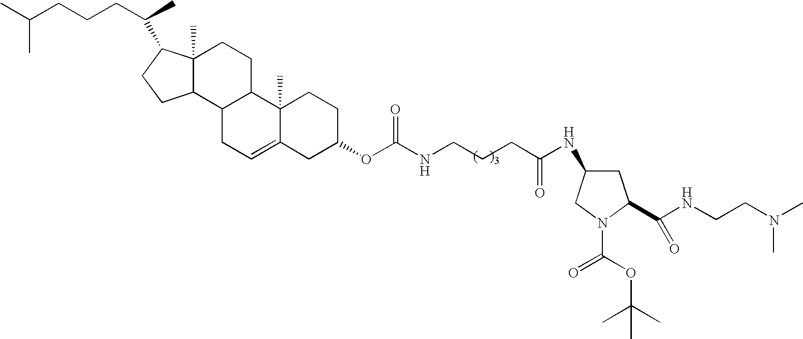

55

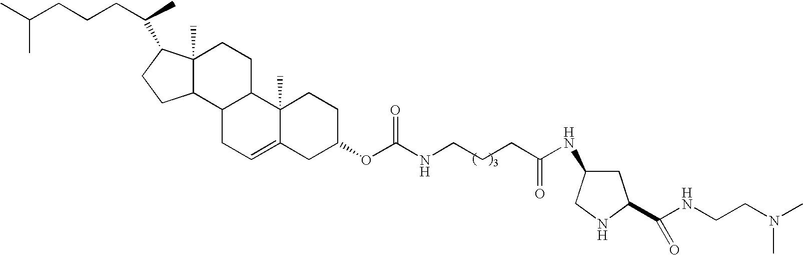

56

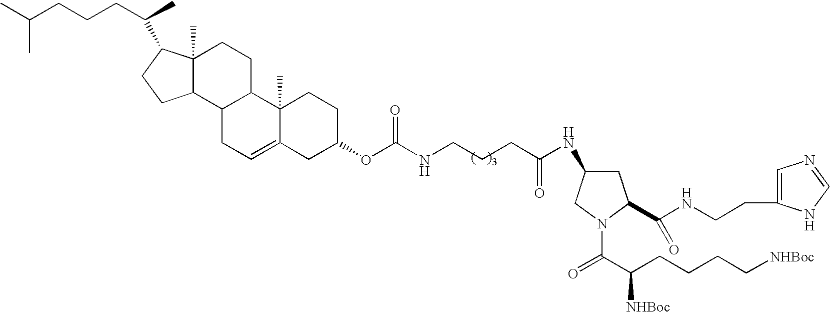

57

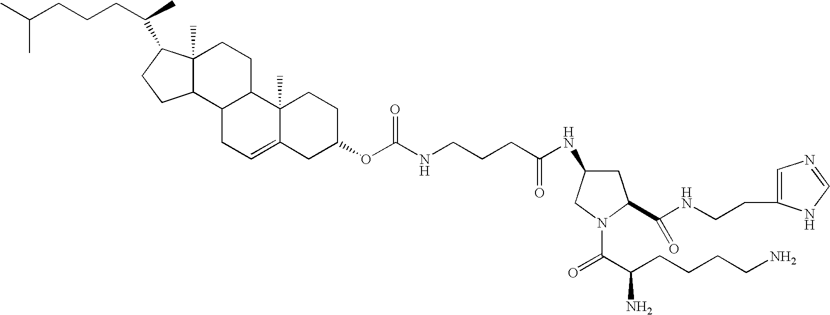

58

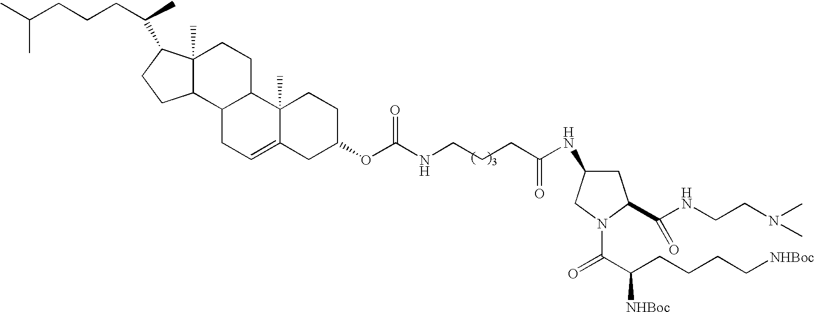

59

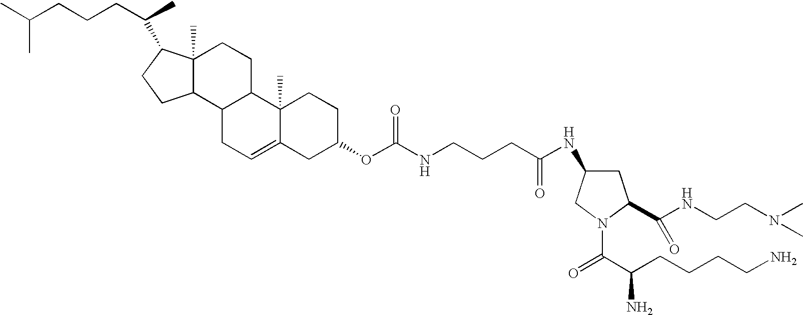

60

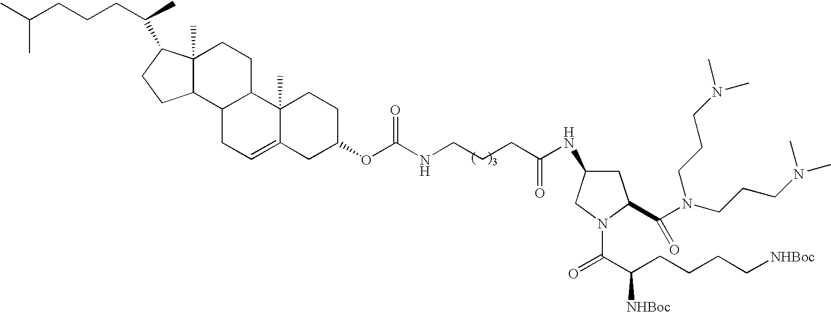

61

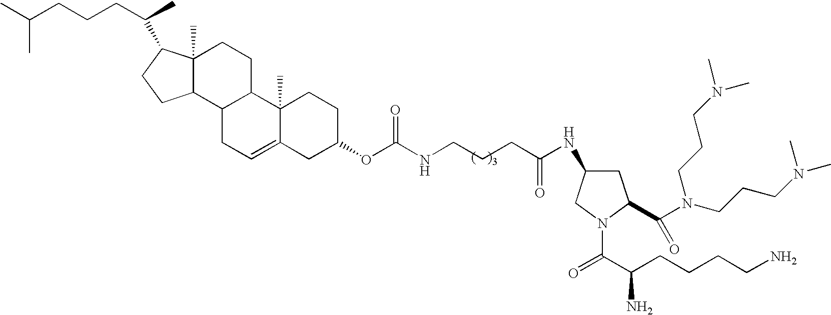

62

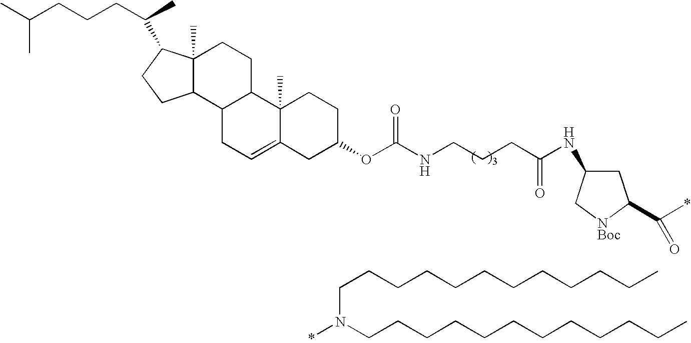

63

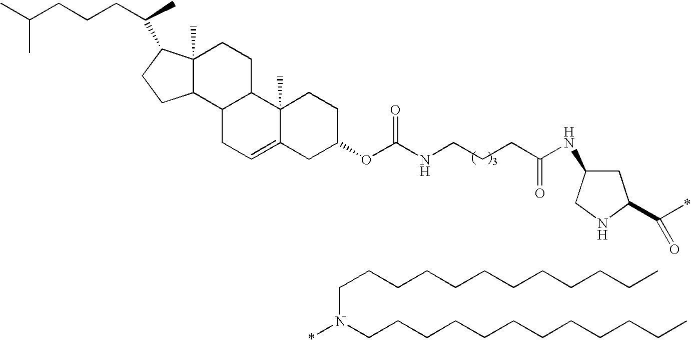

64

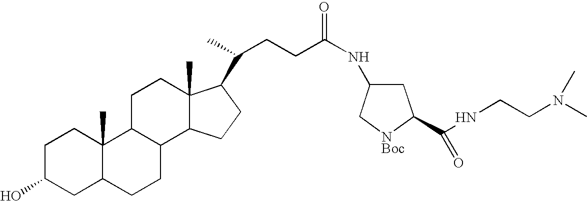

65

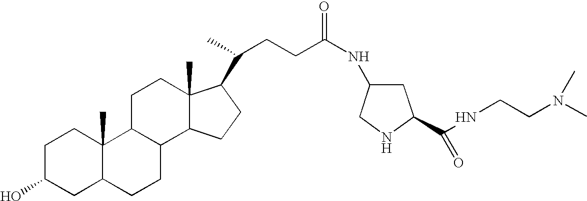

66

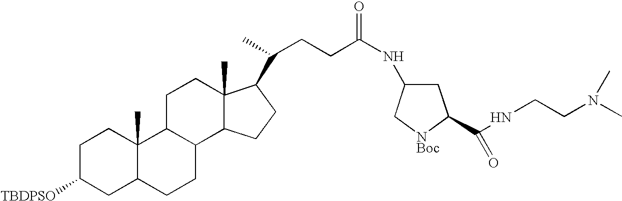

67

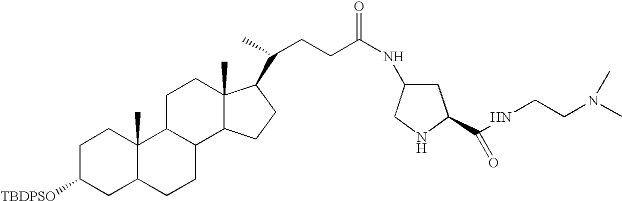

68

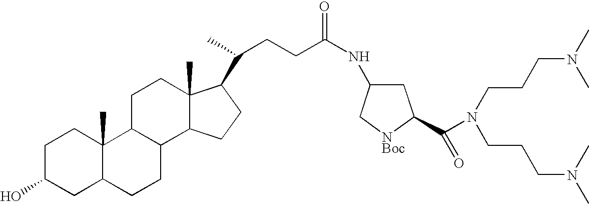

69

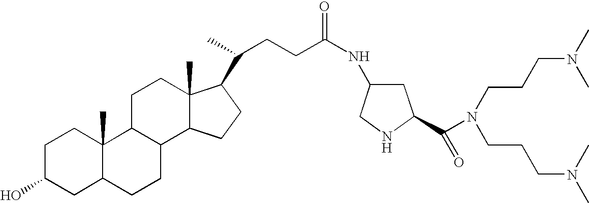

70

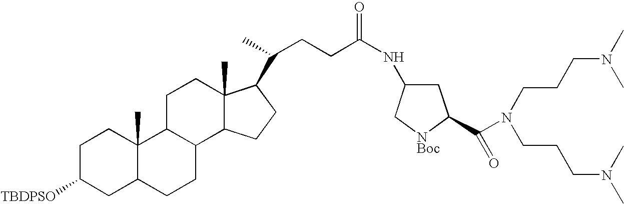

71

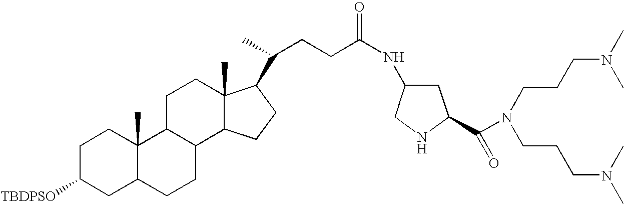

72

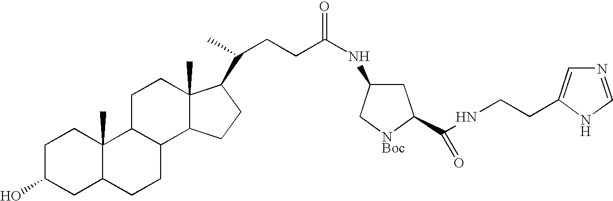

73

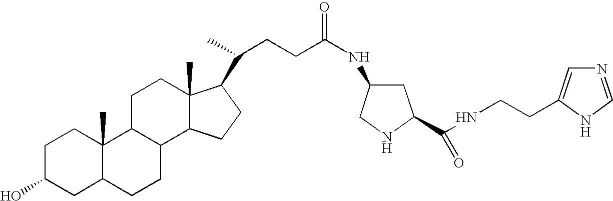

74

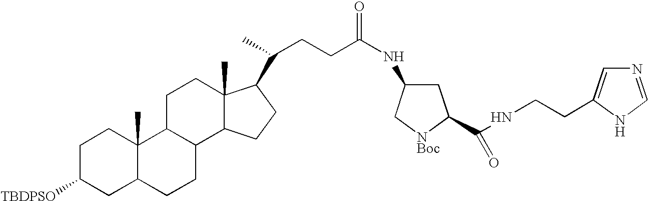

75

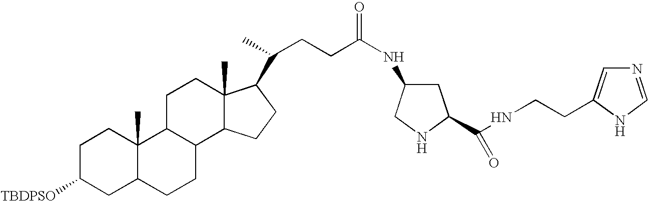

76

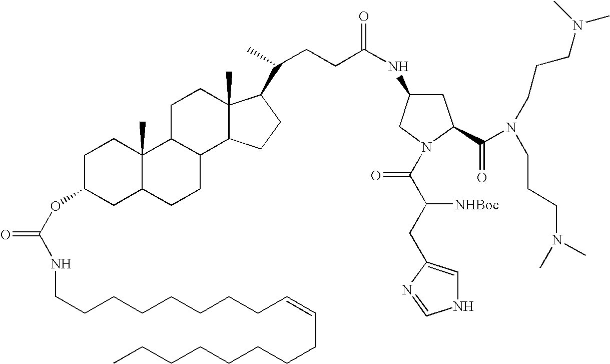

77

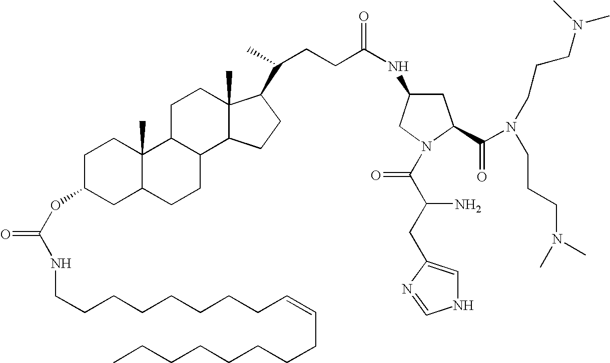

78

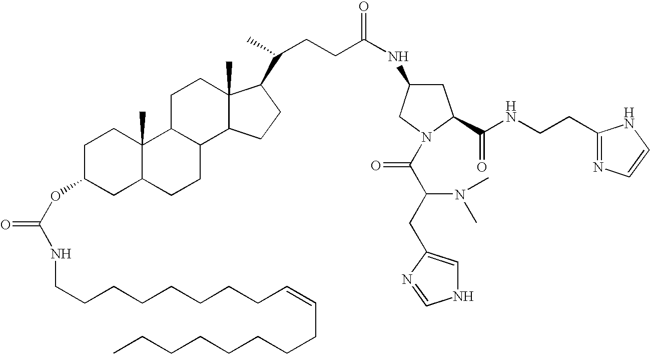

79

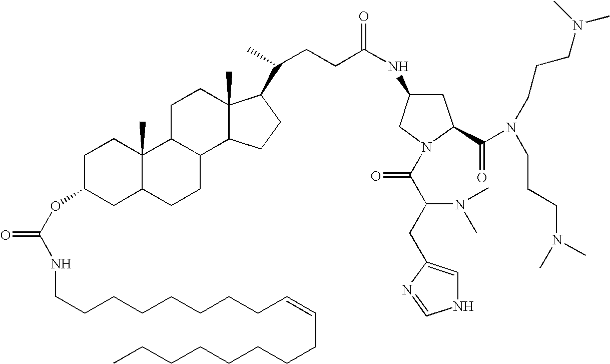

80

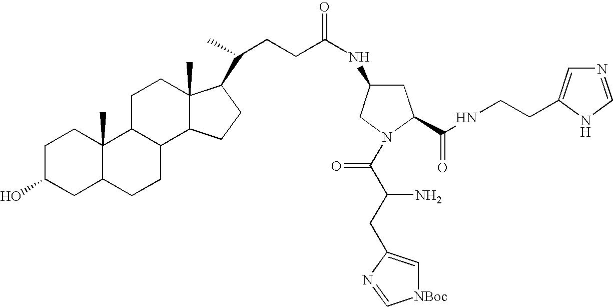

81

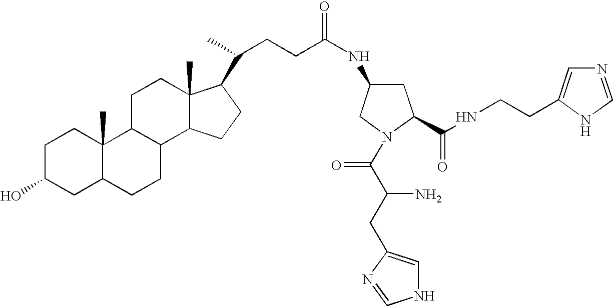

82

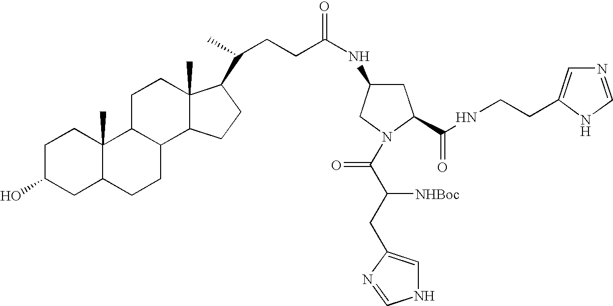

83

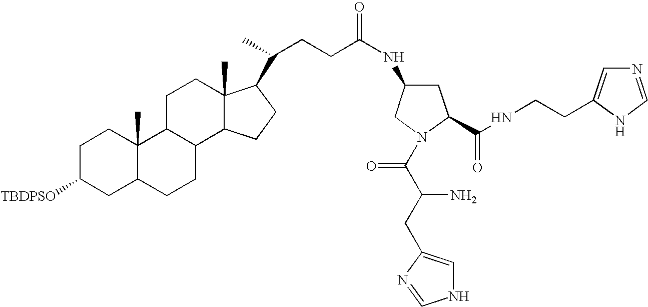

84

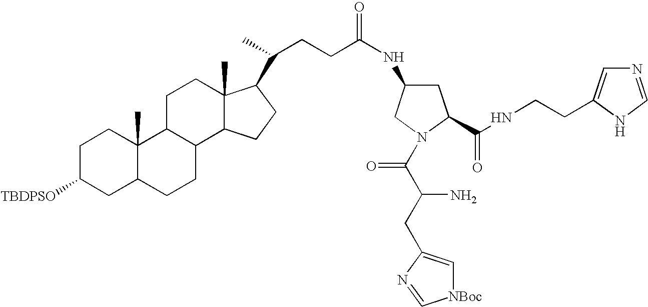

85

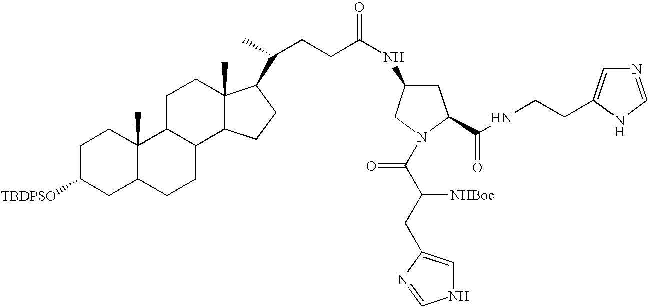

86

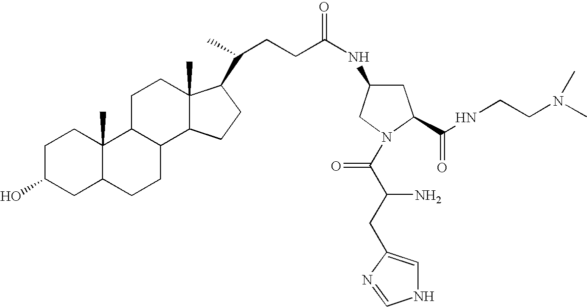

87

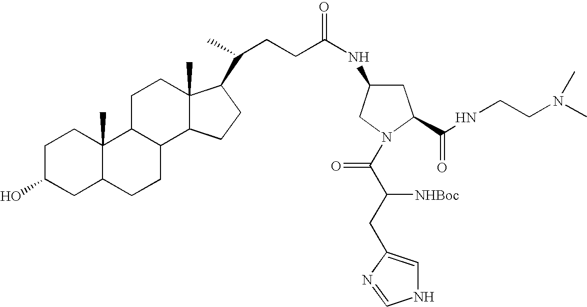

88

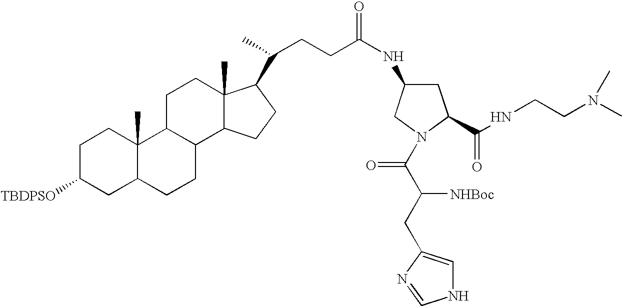

89

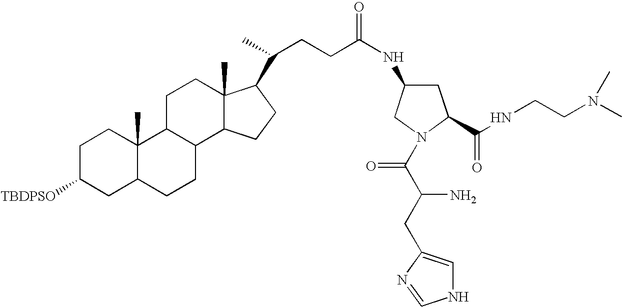

90

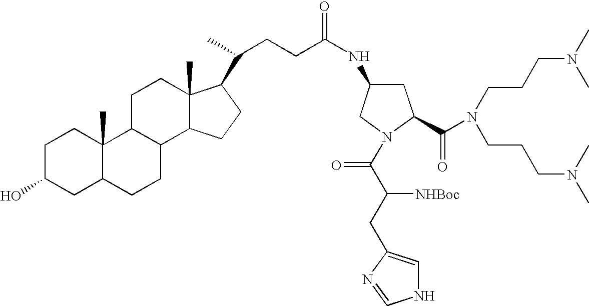

91

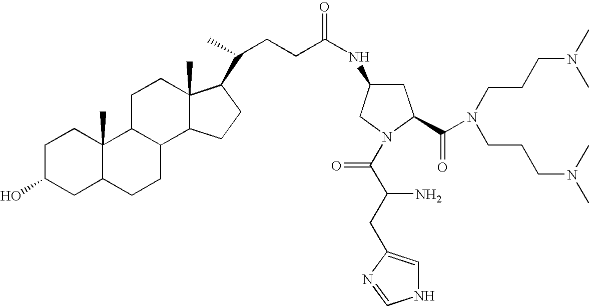

92

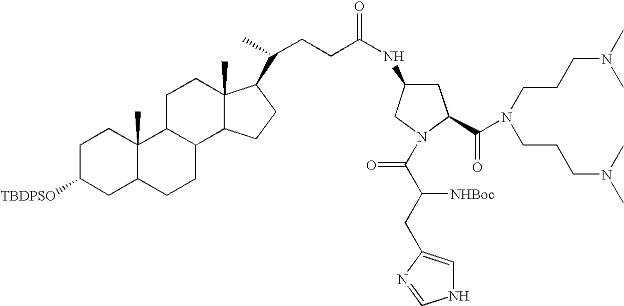

93

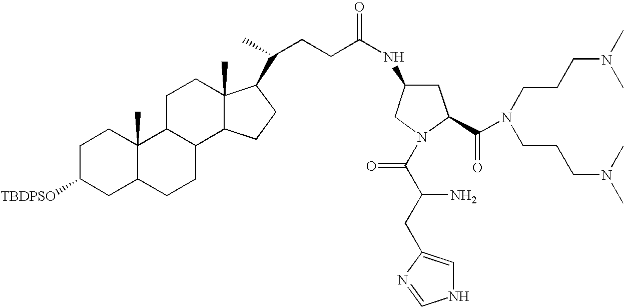

94

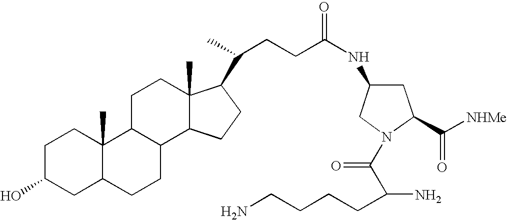

95

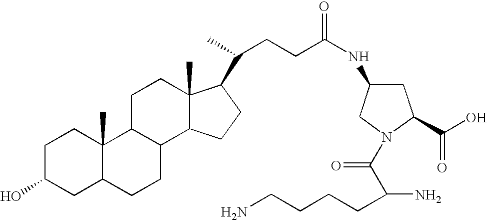

96

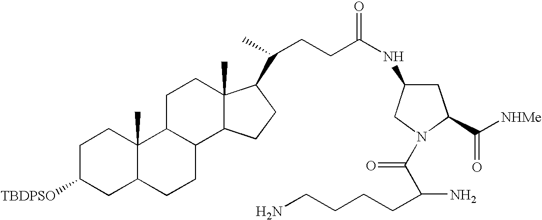

97

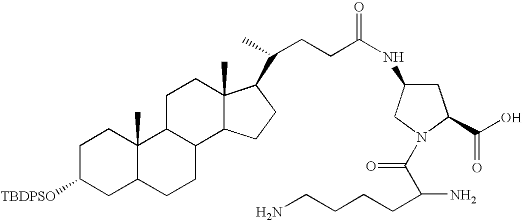

98

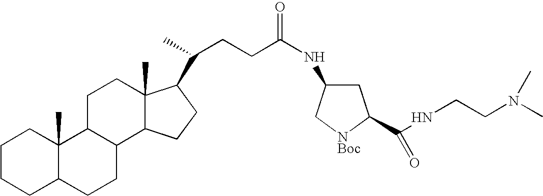

99

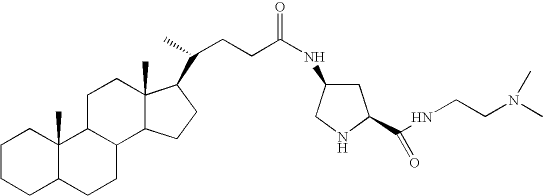

100

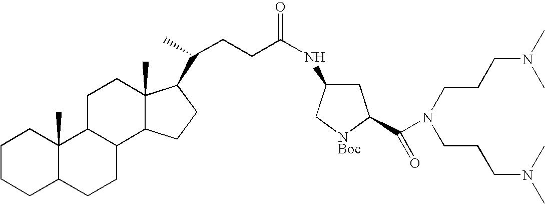

101

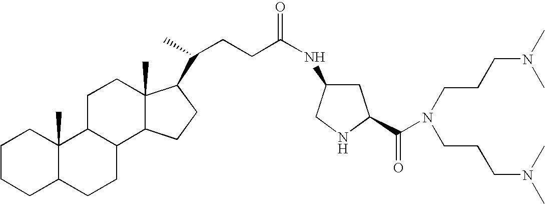

102

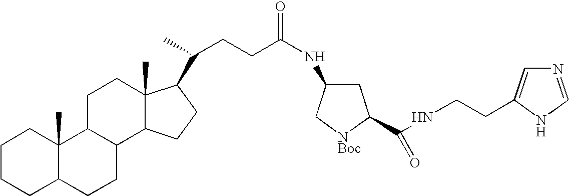

103

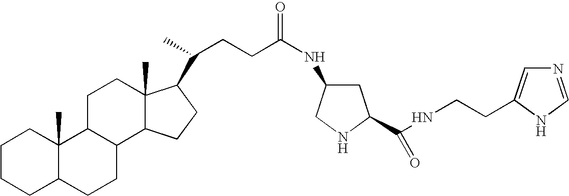

104

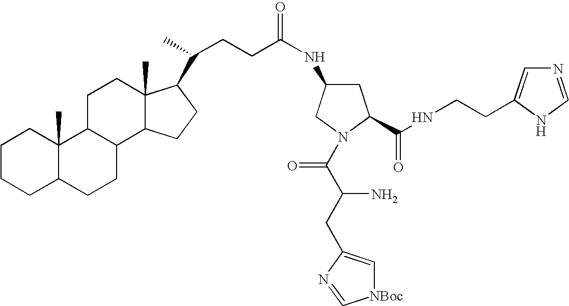

105

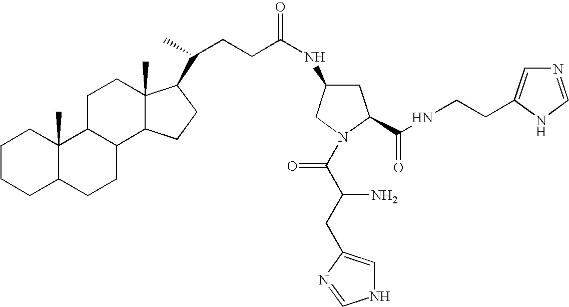

106

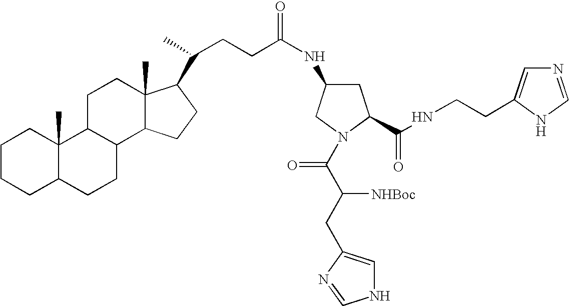

107

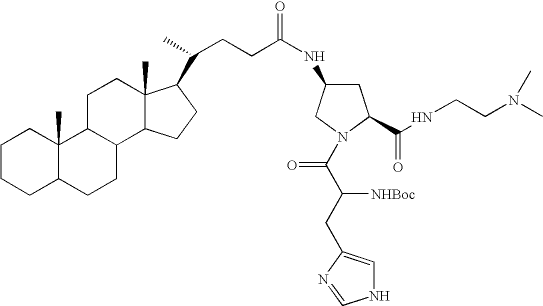

108

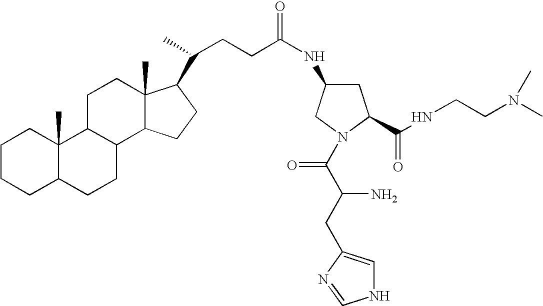

109

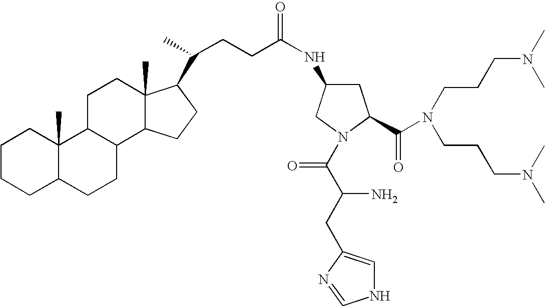

110

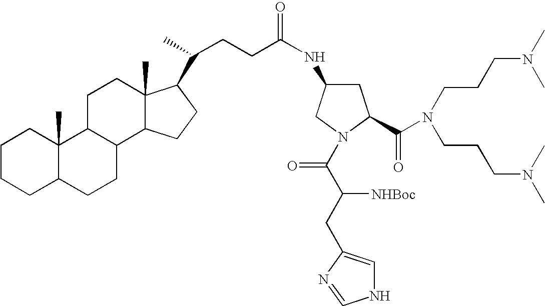

111

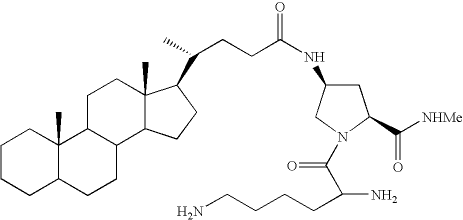

112

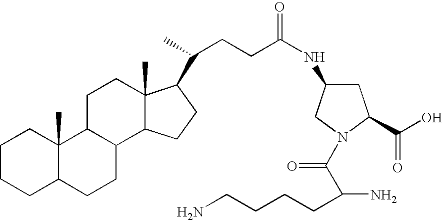

113

TABLE 2
Stock solution of selected cationic lipid hydrochloride salt^{a} for siRNA
transfection

| Notebook | |
|---|---|
| ID | Compound structure |
| 501 | 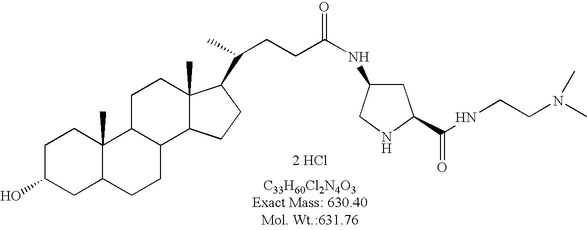 |
| 502 | 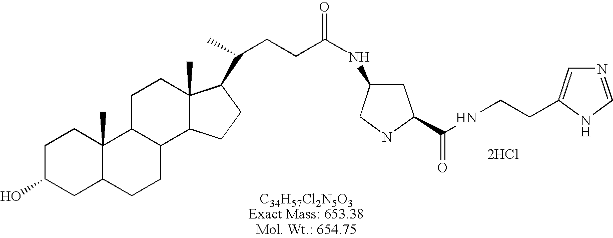 |
| 503 | 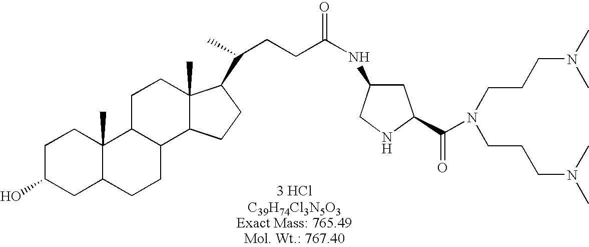 |
| 504 | 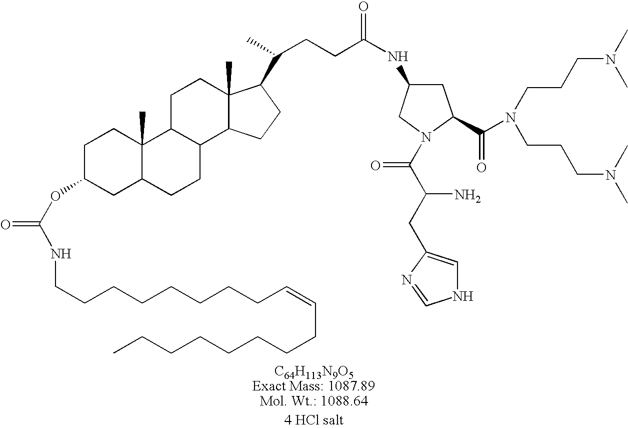 |
| 505 | 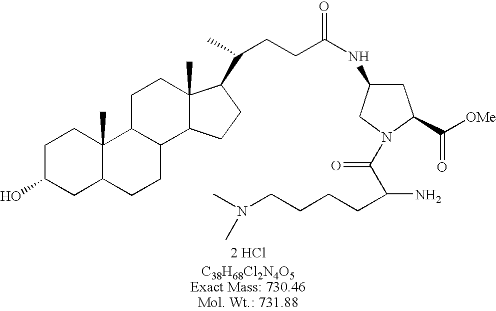 |
| 506 | 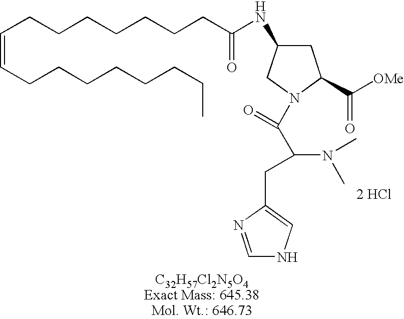 |
| 507 | 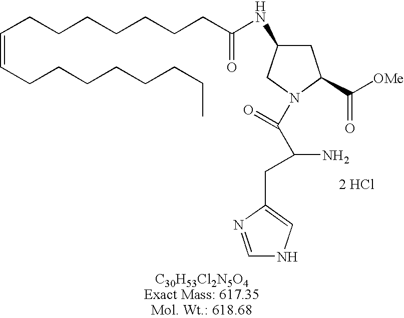 |
| 508 | 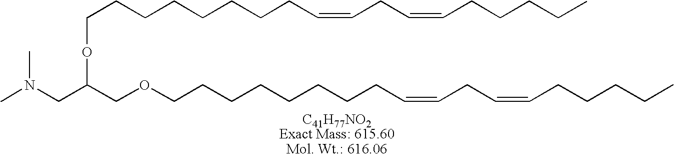 |
| 509 | 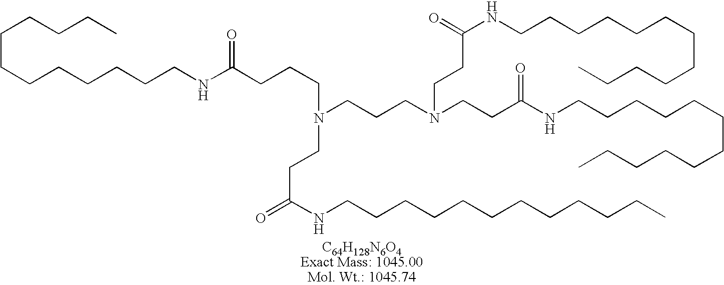 |
| 510 | 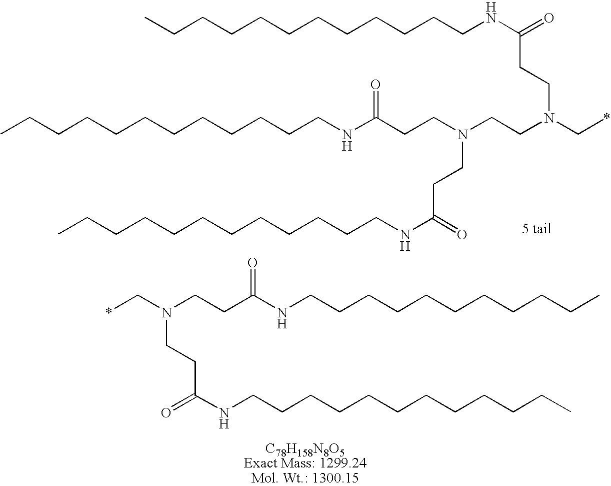 |
| 511 | 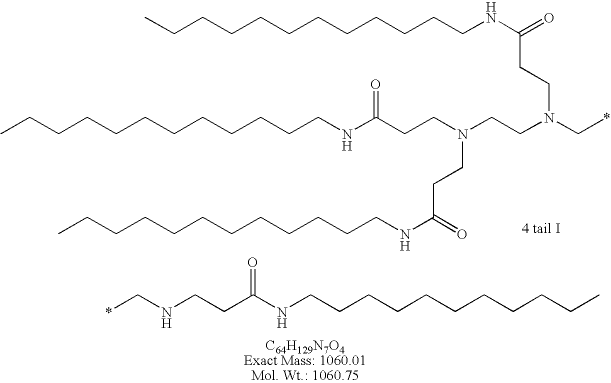 |
| 512 | 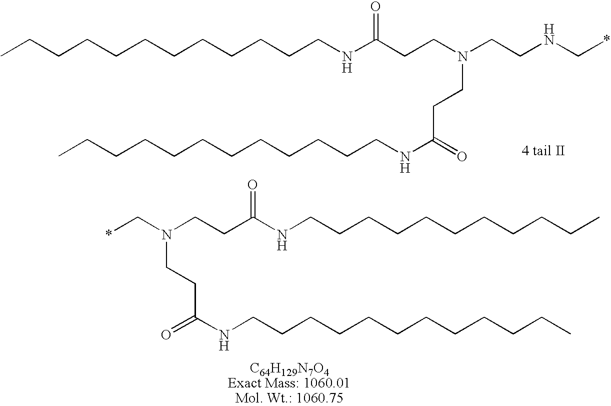 |
| 513 | 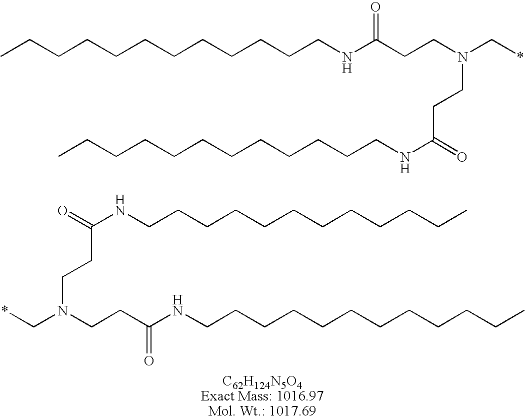 |
| 514 | 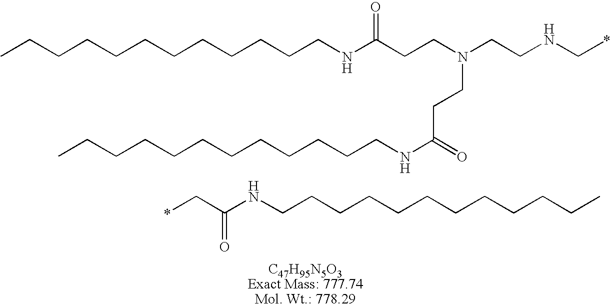 |
| 515 | 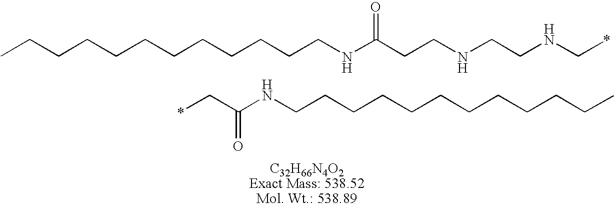 |
| 516 | 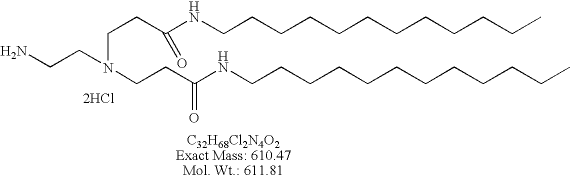 |
| 517 | 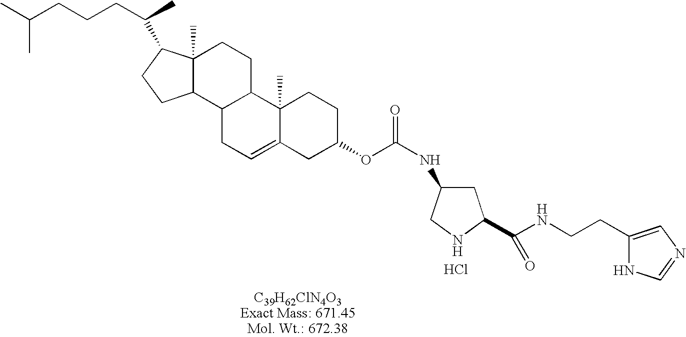 |
| 518 | 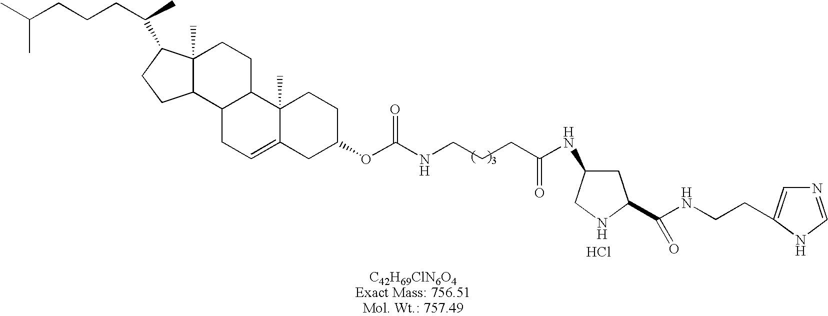 |
| 519 | 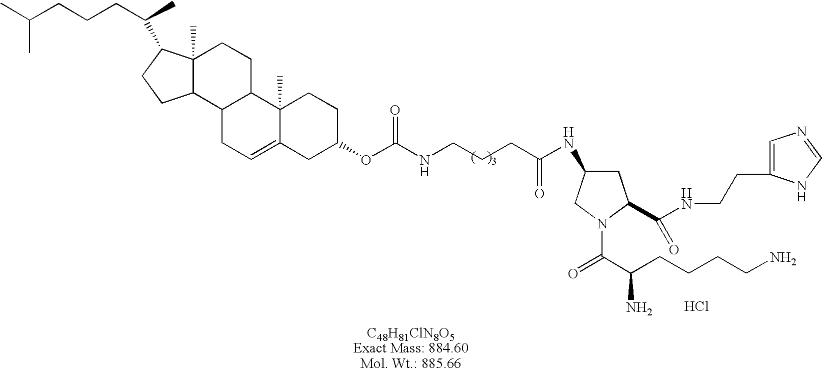 |
| 520 | 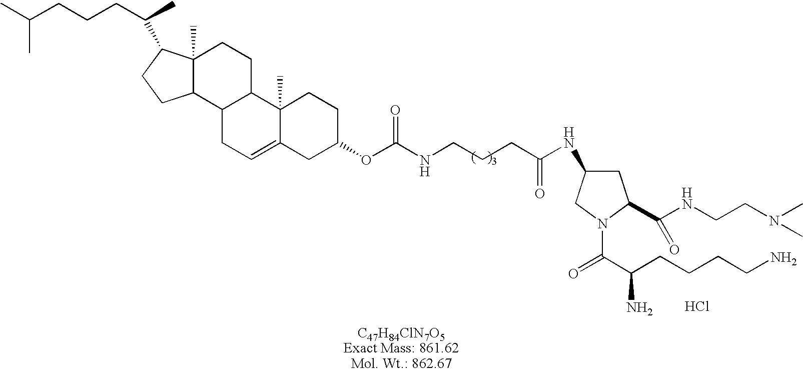 |
| 521 | 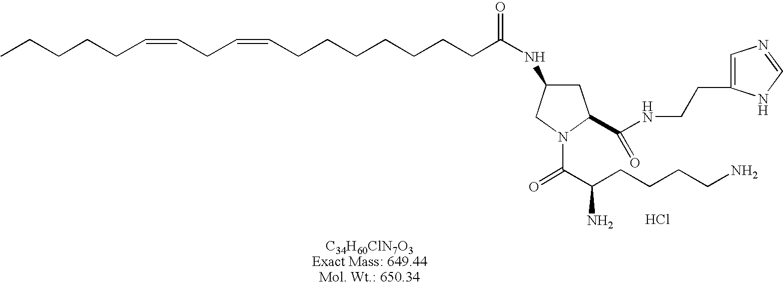 |
| 522 | 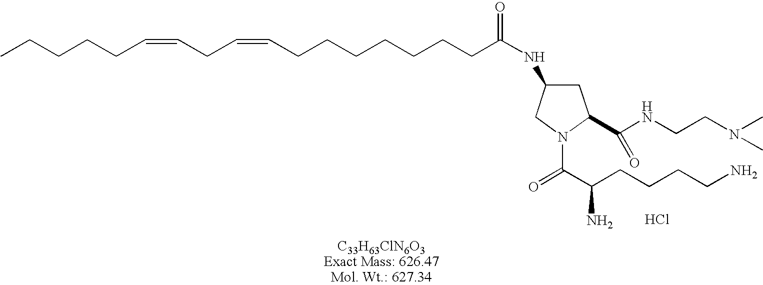 |
| 523 | 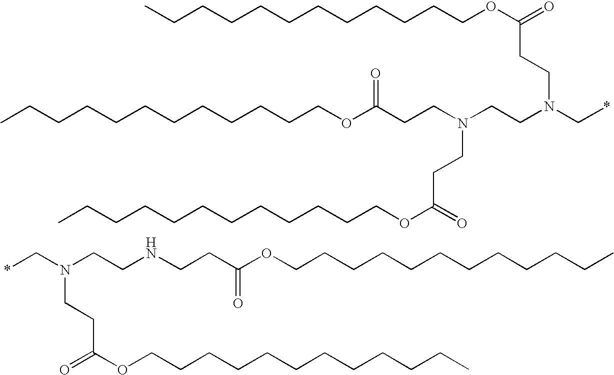 |

| | | | | 40 Equivalent |
|---|---|---|---|---|
| | | | | volume^{d} |
| | | | | (10 mM RNA, |
| | | | Charge | 1 mL) |
| Notebook | C | C^{b} | Equiv.^{c} | 1:1 Charge |
| ID | (mg/mL) | (mM) | (Normality) | ratio |
| 501 | 33.3 | 52.82 | 105.64 | 3.79 |
| 502 | 50.0 | 76.53 | 153.05 | 2.61 |
| 503 | 50.0 | 65.32 | 195.95 | 2.04 |
| 504 | 50.0 | 45.96 | 183.84 | 2.18 |
| 505 | 50.0 | 68.45 | 136.90 | 2.92 |
| 506 | 50.0 | 77.35 | 154.71 | 2.59 |
| 507 | 50.0 | 80.99 | 161.98 | 2.47 |
| 508 | 50.0 | 76.22 | 76.22 | 5.25 |
| 509 | 33.3 | 31.87 | 63.73 | 6.28 |
| 510 | 33.3 | 25.63 | 76.89 | 5.20 |
| 511 | 50.0 | 47.20 | 141.50 | 2.83 |
| 512 | 50.0 | 47.20 | 141.50 | 2.83 |
| 513 | 5.0 | 4.92 | 9.83 | 40.68 |
| 514 | 50.0 | 64.29 | 128.58 | 3.11 |
| 515 | 33.3 | 62.21 | 124.43 | 3.21 |
| 516 | 5.0 | 8.19 | 16.38 | 24.42 |
| 517 | 10.0 | 14.89 | 29.79 | 13.42 |
| 518 | 20.0 | 26.44 | 52.87 | 7.57 |
| 519 | 50.0 | 56.52 | 169.56 | 2.36 |
| 520 | 50.0 | 58.03 | 174.09 | 2.30 |
| 521 | 50.0 | 77.00 | 230.97 | 1.73 |
| 522 | 33.3 | 53.15 | 159.46 | 2.51 |
| 523 | | | | |

^{a}Hydrochloride salt was prepared by treatment with excess HCl in ether and subsequent removal of excess HCl and evaporation of solvents, drying under vacuum overnight.
^{b}Molality in mMol (Column #3) = (wt of compound/mol. wt of the compound) × (1000 mL/VmL) = no of mMol.
^{c}Normality of solution (Charge equivalent) =: (wt of compound/mol. wt of the compound) × (1000 mL/VmL) × (total number of protonatable nitrogen.
^{d}Column 6 (Volume required to make/obtain 1:1 charge ratio for 1 mL stock 10 mM siRNA): N1 × V1 = N2 × V2; V1 = (N2 × V2)/V1 {(10 × 40) × 1}/(value value from column 5); where N2 and V2 are the normality of 10 mM siRNA and volume of stock solution.

### Methods of Making Cationic Lipid Compounds and Cationic Lipid Containing Preparations

The compounds described herein can be obtained from commercial sources (e.g., Asinex, Moscow, Russia; Bionet, Camelford, England; ChemDiv, SanDiego, Calif.; Comgenex, Budapest, Hungary; Enamine, Kiev, Ukraine; IF Lab, Ukraine; Interbioscreen, Moscow, Russia; Maybridge, Tintagel, UK; Specs, The Netherlands; Timtec, Newark, Del.; Vitas-M Lab, Moscow, Russia) or synthesized by conventional methods as shown below using commercially available starting materials and reagents.

Methods of Making Cyclic Lipids

The cationic lipids described are prepared either from diastereomerically pure or racemic 4-aminoaminioproline or its analogues. In general, selective mono or dialkylation or amidation of the exocyclic amine of 4-aminoproline with liophilic molecules or moieties constitute the lipid chain of the cationic lipid. A second lipophilic components is linked to the carboxyl group of 4-aminoproline via amide or ester linkage. A cationic or head group with broader pKa distribution is attached either to the ring nitrogen or to the carboxyl or both via alkylation, amidation or esterification as appropriate to the lipid of interest. Interchange of lipid components and cationic moieties between the functional groups affords isomeric cationic lipids. Attachment of the lipid moieties to the ring nitrogen and the cationic moieties to the exocyclic amine and vice versa affords two set of isomers. Similarly interchanging of substituents between the ring nitrogen and carboxyl group and between carboxyl and exocyclic amine afford other sets of isomeric lipids.

Upon completion of the reaction, one or more products can be isolated from the reaction mixture. For example, a compound can be isolated as a single product (e.g., a single structural isomer) or as a mixture of product (e.g., a plurality of structural isomers and/or a plurality of compounds. In some embodiments, one or more reaction products can be isolated and/or purified using chromatography, such as flash chromatography, gravity chromatography (e.g., gravity separation of isomers using silica gel), column chromatography (e.g., normal phase HPLC or RPHPLC), or moving bed chromatography. In some embodiments, a reaction product is purified to provide a preparation containing at least about 80% of a single compound, such as a single structural isomer (e.g., at least about 85%, at least about 90%, at least about 95%, at least about 97%, at least about 99%).

In some embodiments, a free amine product is treated with an acid such as HCl to prove an amine salt of the product (e.g., a hydrochloride salt). In some embodiments a salt product provides improved properties, e.g., for handling and/or storage, relative to the corresponding free amine product. In some embodiments, a salt product can prevent or reduce the rate of formation of breakdown product such as N-oxide or N-carbonate formation relative to the corresponding free amine. In some embodiments, a salt product can have improved properties for use in a therapeutic formulation relative to the corresponding free amine.

In some embodiments, the reaction mixture is further treated, for example, to purify one or more products or to remove impurities such as unreacted starting materials. In some embodiments the reaction mixture is treated with an immobilized (e.g., polymer bound) thiol moiety, which can trap unreacted acrylamide. In some embodiments, an isolated product can be treated to further remove impurities, e.g., an isolated product can be treated with an immobilized thiol moiety, trapping unreacted acrylamide compounds.

In some embodiments a reaction product can be treated with an immobilized (e.g., polymer bound) isothiocyanate. For example, a reaction product including tertiary amines can be treated with an immobilized isothiocyanate to remove primary and/or secondary amines from the product.

### Association Complexes

The lipid compounds and lipid preparations described herein can be used as a component in an association complex, for example a liposome or a lipoplex. Such association complexes can be used to administer a nucleic acid based therapy such as an RNA, for example a single stranded or double stranded RNA such as dsRNA.

The association complexes disclosed herein can be useful for packaging an oligonucleotide agent capable of modifying gene expression by targeting and binding to a nucleic acid. An oligonucleotide agent can be single-stranded or double-stranded, and can include, e.g., a dsRNA, a pre-mRNA, an mRNA, a microRNA (miRNA), a mi-RNA precursor (pre-miRNA), plasmid or DNA, or to a protein. An oligonucleotide agent featured in the invention can be, e.g., a dsRNA, a microRNA, antisense RNA, antagomir, decoy RNA, DNA, plasmid and aptamer.

Association complexes can include a plurality of components. In some embodiments, an association complex such as a liposome can include an active ingredient such as a nucleic acid therapeutic (such as an oligonucleotide agent, e.g., dsRNA), a cationic lipid such as a lipid described herein. In some embodiments, the association complex can include a plurality of therapeutic agents, for example two or three single or double stranded nucleic acid moieties targeting more than one gene or different regions of the same gene. Other components can also be included in an association complex, including a PEG-lipid such as a PEG-lipid described herein, or a structural component, such as cholesterol. In some embodiments the association complex also includes a fusogenic lipid or component and/or a targeting molecule. In some preferred embodiments, the association complex is a liposome including an oligonucleotide agent such as dsRNA, a cyclic lipid described herein such as a compound of formula (I), (II), (III), or (IV), a PEG-lipid such as a PEG-lipid described herein, and a structural component such as cholesterol.

### Single Stranded Ribonucleid Acid

Oligonucleotide agents include microRNAs (miRNAs). MicroRNAs are small noncoding RNA molecules that are capable of causing post-transcriptional silencing of specific genes in cells such as by the inhibition of translation or through degradation of the targeted mRNA. A miRNA can be completely complementary or can have a region of noncomplementarity with a target nucleic acid, consequently resulting in a “bulge” at the region of non-complementarity. The region of noncomplementarity (the bulge) can be flanked by regions of sufficient complementarity, preferably complete complementarity to allow duplex formation. Preferably, the regions of complementarity are at least 8 to 10 nucleotides long (e.g., 8, 9, or 10 nucleotides long). A miRNA can inhibit gene expression by repressing translation, such as when the microRNA is not completely complementary to the target nucleic acid, or by causing target RNA degradation, which is believed to occur only when the miRNA binds its target with perfect complementarity. The invention also can include double-stranded precursors of miRNAs that may or may not form a bulge when bound to their targets.

In a preferred embodiment an oligonucleotide agent featured in the invention can target an endogenous miRNA or pre-miRNA. The oligonucleotide agent featured in the invention can include naturally occurring nucleobases, sugars, and covalent internucleotide (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for the endogenous miRNA target, and/or increased stability in the presence of nucleases. An oligonucleotide agent designed to bind to a specific endogenous miRNA has substantial complementarity, e.g., at least 70, 80, 90, or 100% complementary, with at least 10, 20, or 25 or more bases of the target miRNA.

A miRNA or pre-miRNA can be 16-100 nucleotides in length, and more preferably from 16-80 nucleotides in length. Mature miRNAs can have a length of 16-30 nucleotides, preferably 21-25 nucleotides, particularly 21, 22, 23, 24, or 25 nucleotides. MicroRNA precursors can have a length of 70-100 nucleotides and have a hairpin conformation. MicroRNAs can be generated in vivo from pre-miRNAs by enzymes called Dicer and Drosha that specifically process long pre-miRNA into functional miRNA. The microRNAs or precursor mi-RNAs featured in the invention can be synthesized in vivo by a cell-based system or can be chemically synthesized. MicroRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting. Methods of synthesis and chemical modifications are described in greater detail below.

Given a sense strand sequence (e.g., the sequence of a sense strand of a cDNA molecule), a miRNA can be designed according to the rules of Watson and Crick base pairing. The miRNA can be complementary to a portion of an RNA, e.g., a miRNA, a pre-miRNA, a pre-mRNA or an mRNA. For example, the miRNA can be complementary to the coding region or noncoding region of an mRNA or pre-mRNA, e.g., the region surrounding the translation start site of a pre-mRNA or mRNA, such as the 5′ UTR. A miRNA oligonucleotide can be, for example, from about 12 to 30 nucleotides in length, preferably about 15 to 28 nucleotides in length (e.g., 16, 17, 18, 19, 20, 21, 22, 23, 24, or 25 nucleotides in length).

In particular, a miRNA or a pre-miRNA featured in the invention can have a chemical modification on a nucleotide in an internal (i.e., non-terminal) region having noncomplementarity with the target nucleic acid. For example, a modified nucleotide can be incorporated into the region of a miRNA that forms a bulge. The modification can include a ligand attached to the miRNA, e.g., by a linker (e.g., see diagrams OT-I through OT-IV below). The modification can, for example, improve pharmacokinetics or stability of a therapeutic miRNA, or improve hybridization properties (e.g., hybridization thermodynamics) of the miRNA to a target nucleic acid. In some embodiments, it is preferred that the orientation of a modification or ligand incorporated into or tethered to the bulge region of a miRNA is oriented to occupy the space in the bulge region. For example, the modification can include a modified base or sugar on the nucleic acid strand or a ligand that functions as an intercalator. These are preferably located in the bulge. The intercalator can be an aromatic, e.g., a polycyclic aromatic or heterocyclic aromatic compound. A polycyclic intercalator can have stacking capabilities, and can include systems with 2, 3, or 4 fused rings. The universal bases described below can be incorporated into the miRNAs. In some embodiments, it is preferred that the orientation of a modification or ligand incorporated into or tethered to the bulge region of a miRNA is oriented to occupy the space in the bulge region. This orientation facilitates the improved hybridization properties or an otherwise desired characteristic of the miRNA.

In one embodiment, an miRNA or a pre-miRNA can include an aminoglycoside ligand, which can cause the miRNA to have improved hybridization properties or improved sequence specificity. Exemplary aminoglycosides include glycosylated polylysine; galactosylated polylysine; neomycin B; tobramycin; kanamycin A; and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-S-acridine has an increased affinity for the HIV Rev-response element (RRE). In some embodiments the guanidine analog (the guanidinoglycoside) of an aminoglycoside ligand is tethered to an oligonucleotide agent. In a guanidinoglycoside, the amine group on the amino acid is exchanged for a guanidine group. Attachment of a guanidine analog can enhance cell permeability of an oligonucleotide agent.

In one embodiment, the ligand can include a cleaving group that contributes to target gene inhibition by cleavage of the target nucleic acid. Preferably, the cleaving group is tethered to the miRNA in a manner such that it is positioned in the bulge region, where it can access and cleave the target RNA. The cleaving group can be, for example, a bleomycin (e.g., bleomycin-A₅, bleomycin-A₂, or bleomycin-B₂), pyrene, phenanthroline (e.g., O-phenanthroline), a polyamine, a tripeptide (e.g., lys-tyr-lys tripeptide), or metal ion chelating group. The metal ion chelating group can include, e.g., a Lu(III) or EU(III) or Gd(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a miRNA or a pre-miRNA to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. The methods and compositions featured in the invention include miRNAs that inhibit target gene expression by a cleavage or non-cleavage dependent mechanism.

A miRNA or a pre-miRNA can be designed and synthesized to include a region of noncomplementarity (e.g., a region that is 3, 4, 5, or 6 nucleotides long) flanked by regions of sufficient complementarity to form a duplex (e.g., regions that are 7, 8, 9, 10, or 111 nucleotides long).

For increased nuclease resistance and/or binding affinity to the target, the miRNA sequences can include 2′-O-methyl, 2′-fluorine, 2′-O-methoxyethyl, 2′-O-aminopropyl, 2′-amino, and/or phosphorothioate linkages. Inclusion of locked nucleic acids (LNA), 2-thiopyrimidines (e.g., 2-thio-U), 2-amino-A, G-clamp modifications, and ethylene nucleic acids (ENA), e.g., 2′-4′-ethylene-bridged nucleic acids, can also increase binding affinity to the target. The inclusion of furanose sugars in the oligonucleotide backbone can also decrease endonucleolytic cleavage. A miRNA or a pre-miRNA can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′ end of oligonucleotide. Even small alkyl chains, aryl groups, or heterocyclic conjugates or modified sugars (D-ribose, deoxyribose, glucose etc.) can block 3′-5′-exonucleases.

The 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of oligonucleotide. Even small alkyl chains, aryl groups, or heterocyclic conjugates or modified sugars (D-ribose, deoxyribose, glucose etc.) can block 3′-5′-exonucleases.

In one embodiment, a miRNA or a pre-miRNA includes a modification that improves targeting, e.g. a targeting modification described herein. Examples of modifications that target miRNA molecules to particular cell types include carbohydrate sugars such as galactose, N-acetylgalactosamine, mannose; vitamins such as folates, biotin, vitamin E; other ligands such as RGDs and RGD mimics; and small molecules including naproxen, ibuprofen or other known protein-binding molecules.

A miRNA or a pre-miRNA can be constructed using chemical synthesis and/or enzymatic ligation reactions using procedures known in the art. For example, a miRNA or a pre-miRNA can be chemically synthesized using naturally occurring nucleotides or variously modified nucleotides designed to increase the biological stability of the molecules or to increase the physical stability of the duplex formed between the miRNA or a pre-miRNA and target nucleic acids, e.g., phosphorothioate derivatives and acridine substituted nucleotides can be used. Other appropriate nucleic acid modifications are described herein. Alternatively, the miRNA or pre-miRNA nucleic acid can be produced biologically using an expression vector into which a nucleic acid has been subcloned in an antisense orientation (i.e., RNA transcribed from the inserted nucleic acid will be of an antisense orientation to a target nucleic acid of interest).

### Antisense-Type Oligonucleotide Agents

The single-stranded oligonucleotide agents featured in the invention include antisense nucleic acids. An “antisense” nucleic acid includes a nucleotide sequence that is complementary to a “sense” nucleic acid encoding a gene expression product, e.g., complementary to the coding strand of a double-stranded cDNA molecule or complementary to an RNA sequence, e.g., a pre-mRNA, mRNA, miRNA, or pre-miRNA. Accordingly, an antisense nucleic acid can form hydrogen bonds with a sense nucleic acid target.

Given a coding strand sequence (e.g., the sequence of a sense strand of a cDNA molecule), antisense nucleic acids can be designed according to the rules of Watson and Crick base pairing. The antisense nucleic acid molecule can be complementary to a portion of the coding or noncoding region of an RNA, e.g., a pre-mRNA or mRNA. For example, the antisense oligonucleotide can be complementary to the region surrounding the translation start site of a pre-mRNA or mRNA, e.g., the 5′ UTR. An antisense oligonucleotide can be, for example, about 10 to 25 nucleotides in length (e.g., 11, 12, 13, 14, 15, 16, 18, 19, 20, 21, 22, 23, or 24 nucleotides in length). An antisense oligonucleotide can also be complementary to a miRNA or pre-miRNA.

An antisense nucleic acid can be constructed using chemical synthesis and/or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and target nucleic acids, e.g., phosphorothioate derivatives and acridine substituted nucleotides can be used. Other appropriate nucleic acid modifications are described herein. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

An antisense agent can include ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA, and prevent access of the translation machinery to the target RNA transcript, thereby preventing protein synthesis. An antisense molecule including only deoxyribonucleotides, or deoxyribonucleotides and ribonucleotides, e.g., DNA sequence flanked by RNA sequence at the 5′ and 3′ ends of the antisense agent, can hybridize to a complementary RNA, and the RNA target can be subsequently cleaved by an enzyme, e.g., RNAse H. Degradation of the target RNA prevents translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

For increased nuclease resistance, an antisense agent can be further modified by inverting the nucleoside at the 3′-terminus with a 3′-3′ linkage. In another alternative, the 3′-terminus can be blocked with an aminoalkyl group.

In one embodiment, an antisense oligonucleotide agent includes a modification that improves targeting, e.g. a targeting modification described herein.

### Decoy-Type Oligonucleotide Agents

An oligonucleotide agent featured in the invention can be a decoy nucleic acid, e.g., a decoy RNA. A decoy nucleic acid resembles a natural nucleic acid, but is modified in such a way as to inhibit or interrupt the activity of the natural nucleic acid. For example, a decoy RNA can mimic the natural binding domain for a ligand. The decoy RNA therefore competes with natural binding target for the binding of a specific ligand. The natural binding target can be an endogenous nucleic acid, e.g., a pre-miRNA, miRNA, premRNA, mRNA or DNA. For example, it has been shown that over-expression of HIV trans-activation response (TAR) RNA can act as a “decoy” and efficiently bind HIV tat protein, thereby preventing it from binding to TAR sequences encoded in the HIV RNA.

In one embodiment, a decoy RNA includes a modification that improves targeting, e.g. a targeting modification described herein.

The chemical modifications described above for miRNAs and antisense RNAs, and described elsewhere herein, are also appropriate for use in decoy nucleic acids.

### Aptamer-Type Oligonucleotide Agents

An oligonucleotide agent featured in the invention can be an aptamer. An aptamer binds to a non-nucleic acid ligand, such as a small organic molecule or protein, e.g., a transcription or translation factor, and subsequently modifies (e.g., inhibits) activity. An aptamer can fold into a specific structure that directs the recognition of the targeted binding site on the non-nucleic acid ligand. An aptamer can contain any of the modifications described herein.

In one embodiment, an aptamer includes a modification that improves targeting, e.g. a targeting modification described herein.

The chemical modifications described above for miRNAs and antisense RNAs, and described elsewhere herein, are also appropriate for use in decoy nucleic acids.

The details of one or more embodiments of the invention are set forth in the accompanying drawings and the description below. Other features and advantages of the invention will be apparent from the description and drawings, and from the claims. This application incorporates all cited references, patents, and patent applications by references in their entirety for all purposes.

In one aspect, the invention features antagomirs. Antagomirs are single stranded, double stranded, partially double stranded and hairpin structured chemically modified oligonucleotides that target a microRNA.

An antagomir consisting essentially of or comprising at least 12 or more contiguous nucleotides substantially complementary to an endogenous miRNA and more particularly agents that include 12 or more contiguous nucleotides substantially complementary to a target sequence of an miRNA or pre-miRNA nucleotide sequence. Preferably, an antagomir featured in the invention includes a nucleotide sequence sufficiently complementary to hybridize to a miRNA target sequence of about 12 to 25 nucleotides, preferably about 15 to 23 nucleotides. More preferably, the target sequence differs by no more than 1, 2, or 3 nucleotides from a sequence shown in Table 1, and in one embodiment, the antagomir is an agent shown in Table 2a-e. In one embodiment, the antagomir includes a non-nucleotide moiety, e.g., a cholesterol moiety. The non-nucleotide moiety can be attached, e.g., to the 3′ or 5′ end of the oligonucleotide agent. In a preferred embodiment, a cholesterol moiety is attached to the 3′ end of the oligonucleotide agent.

Antagomirs are stabilized against nucleolytic degradation such as by the incorporation of a modification, e.g., a nucleotide modification. In another embodiment, the antagomir includes a phosphorothioate at least the first, second, or third internucleotide linkage at the 5′ or 3′ end of the nucleotide sequence. In yet another embodiment, the antagomir includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In a particularly preferred embodiment, the antagomir includes at least one 2′-O-methyl-modified nucleotide, and in some embodiments, all of the nucleotides of the antagomir include a 2′-O-methyl modification.

An antagomir that is substantially complementary to a nucleotide sequence of an miRNA can be delivered to a cell or a human to inhibit or reduce the activity of an endogenous miRNA, such as when aberrant or undesired miRNA activity, or insufficient activity of a target mRNA that hybridizes to the endogenous miRNA, is linked to a disease or disorder. In one embodiment, an antagomir featured in the invention has a nucleotide sequence that is substantially complementary to miR-122 (see Table 1), which hybridizes to numerous RNAs, including aldolase A mRNA, N-myc downstream regulated gene (Ndrg3) mRNA, IQ motif containing GTPase activating protein-1 (Iqgap1) mRNA, HMG-CoA-reductase (Hmgcr) mRNA, and citrate synthase mRNA and others. In a preferred embodiment, the antagomir that is substantially complementary to miR-122 is antagomir-122 (Table 2a-e). Aldolase A deficiencies have been found to be associated with a variety of disorders, including hemolytic anemia, arthrogryposis complex congenita, pituitary ectopia, rhabdomyolysis, hyperkalemia. Humans suffering from aldolase A deficiencies also experience symptoms that include growth and developmental retardation, midfacial hypoplasia, hepatomegaly, as well as myopathic symptoms. Thus a human who has or who is diagnosed as having any of these disorders or symptoms is a candidate to receive treatment with an antagomir that hybridizes to miR-122.

### Double-Stranded Ribonucleic Acid (dsRNA)

In one embodiment, the invention provides a double-stranded ribonucleic acid (dsRNA) molecule packaged in an association complex, such as a liposome, for inhibiting the expression of a gene in a cell or mammal, wherein the dsRNA comprises an antisense strand comprising a region of complementarity which is complementary to at least a part of an mRNA formed in the expression of the gene, and wherein the region of complementarity is less than 30 nucleotides in length, generally 19-24 nucleotides in length, and wherein said dsRNA, upon contact with a cell expressing said gene, inhibits the expression of said gene by at least 40%. The dsRNA comprises two RNA strands that are sufficiently complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of a gene, the other strand (the sense strand) comprises a region which is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s). The dsRNA can be synthesized by standard methods known in the art as further discussed below, e.g., by use of an automated DNA synthesizer, such as are commercially available from, for example, Biosearch, Applied Biosystems, Inc.

The dsRNAs suitable for packaging in the association complexes described herein can include a duplex structure of between 18 and 25 basepairs (e.g., 21 base pairs). In some embodiments, the dsRNAs include at least one strand that is at least 21 nt long. In other embodiments, the dsRNAs include at least one strand that is at least 15, 16, 17, 18, 19, 20, or more contiguous nucleotides.

The dsRNAs suitable for packaging in the association complexes described herein can contain one or more mismatches to the target sequence. In a preferred embodiment, the dsRNA contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity.

In one embodiment, at least one end of the dsRNA has a single-stranded nucleotide overhang of 1 to 4, generally 1 or 2 nucleotides. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Such dsRNAs have improved stability and inhibitory activity, thus allowing administration at low dosages, i.e., less than 5 mg/kg body weight of the recipient per day. Generally, the antisense strand of the dsRNA has a nucleotide overhang at the 3′-end, and the 5′-end is blunt. In another embodiment, one or more of the nucleotides in the overhang is replaced with a nucleoside thiophosphate.

In yet another embodiment, a dsRNA packaged in an association complex, such as a liposome, is chemically modified to enhance stability. Such nucleic acids may be synthesized and/or modified by methods well established in the art, such as those described in “Current protocols in nucleic acid chemistry”, Beaucage, S. L. et al. (Edrs.), John Wiley & Sons, Inc., New York, N. Y., USA, which is hereby incorporated herein by reference. Chemical modifications may include, but are not limited to 2′ modifications, modifications at other sites of the sugar or base of an oligonucleotide, introduction of non-natural bases into the oligonucleotide chain, covalent attachment to a ligand or chemical moiety, and replacement of internucleotide phosphate linkages with alternate linkages such as thiophosphates. More than one such modification may be employed.

Chemical linking of the two separate dsRNA strands may be achieved by any of a variety of well-known techniques, for example by introducing covalent, ionic or hydrogen bonds; hydrophobic interactions, van der Waals or stacking interactions; by means of metal-ion coordination, or through use of purine analogues. Such chemically linked dsRNAs are suitable for packaging in the association complexes described herein. Generally, the chemical groups that can be used to modify the dsRNA include, without limitation, methylene blue; bifunctional groups, generally bis-(2-chloroethyl)amine; N-acetyl-N′-(p-glyoxylbenzoyl)cystamine; 4-thiouracil; and psoralen. In one embodiment, the linker is a hexa-ethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate groups. The chemical bond at the ends of the dsRNA is generally formed by triple-helix bonds.

In yet another embodiment, the nucleotides at one or both of the two single strands may be modified to prevent or inhibit the degradation activities of cellular enzymes, such as, for example, without limitation, certain nucleases. Techniques for inhibiting the degradation activity of cellular enzymes against nucleic acids are known in the art including, but not limited to, 2′-amino modifications, 2′-amino sugar modifications, 2′-F sugar modifications, 2′-F modifications, 2′-alkyl sugar modifications, 2′-O-alkoxyalkyl modifications like 2′-O-methoxyethyl, uncharged and charged backbone modifications, morpholino modifications, 2′-O-methyl modifications, and phosphoramidate (see, e.g., Wagner, Nat. Med. (1995) 1:1116-8). Thus, at least one 2′-hydroxyl group of the nucleotides on a dsRNA is replaced by a chemical group, generally by a 2′-F or a 2′-O-methyl group. Also, at least one nucleotide may be modified to form a locked nucleotide. Such locked nucleotide contains a methylene bridge that connects the 2′-oxygen of ribose with the 4′-carbon of ribose. Oligonucleotides containing the locked nucleotide are described in Koshkin, A. A., et al., Tetrahedron (1998), 54: 3607-3630) and Obika, S. et al., Tetrahedron Lett. (1998), 39: 5401-5404). Introduction of a locked nucleotide into an oligonucleotide improves the affinity for complementary sequences and increases the melting temperature by several degrees (Braasch, D. A. and D. R. Corey, Chem. Biol. (2001), 8:1-7).

Conjugating a ligand to a dsRNA can enhance its cellular absorption as well as targeting to a particular tissue or uptake by specific types of cells such as liver cells. In certain instances, a hydrophobic ligand is conjugated to the dsRNA to facilitate direct permeation of the cellular membrane and or uptake across the liver cells. Alternatively, the ligand conjugated to the dsRNA is a substrate for receptor-mediated endocytosis. These approaches have been used to facilitate cell permeation of antisense oligonucleotides as well as dsRNA agents. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Li and coworkers report that attachment of folic acid to the 3′-terminus of an oligonucleotide resulted in an 8-fold increase in cellular uptake of the oligonucleotide. Li, S.; Deshmukh, H. M.; Huang, L. Pharm. Res. 1998, 15, 1540. Other ligands that have been conjugated to oligonucleotides include polyethylene glycols, carbohydrate clusters, cross-linking agents, porphyrin conjugates, delivery peptides and lipids such as cholesterol. Other chemical modifications for siRNAs have been described in Manoharan, M. RNA interference and chemically modified small interfering RNAs. Current Opinion in Chemical Biology (2004), 8(6), 570-579.

In certain instances, conjugation of a cationic ligand to oligonucleotides results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed throughout the oligonucleotide. See M. Manoharan Antisense & Nucleic Acid Drug Development 2002, 12, 103 and references therein.

The ligand-conjugated dsRNA of the invention may be synthesized by the use of a dsRNA that bears a pendant reactive functionality, such as that derived from the attachment of a linking molecule onto the dsRNA. This reactive oligonucleotide may be reacted directly with commercially-available ligands, ligands that are synthesized bearing any of a variety of protecting groups, or ligands that have a linking moiety attached thereto. The methods of the invention facilitate the synthesis of ligand-conjugated dsRNA by the use of, in some preferred embodiments, nucleoside monomers that have been appropriately conjugated with ligands and that may further be attached to a solid-support material. Such ligand-nucleoside conjugates, optionally attached to a solid-support material, are prepared according to some preferred embodiments of the methods of the invention via reaction of a selected serum-binding ligand with a linking moiety located on the 5′ position of a nucleoside or oligonucleotide. In certain instances, a dsRNA bearing an aralkyl ligand attached to the 3′-terminus of the dsRNA is prepared by first covalently attaching a monomer building block to a controlled-pore-glass support via a long-chain aminoalkyl group. Then, nucleotides are bonded via standard solid-phase synthesis techniques to the monomer building-block bound to the solid support. The monomer building block may be a nucleoside or other organic compound that is compatible with solid-phase synthesis.

The dsRNA used in the conjugates of the invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

Teachings regarding the synthesis of particular modified oligonucleotides may be found in the following: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; U.S. Pat. Nos. 6,262,241, and 5,459,255, drawn to, inter alia, methods of synthesizing 2′-fluoro-oligonucleotides.

In the ligand-conjugated dsRNA and ligand-molecule bearing sequence-specific linked nucleosides of the invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. Oligonucleotide conjugates bearing a variety of molecules such as steroids, vitamins, lipids and reporter molecules, has previously been described (see Manoharan et al., PCT Application WO 93/07883). In a preferred embodiment, the oligonucleotides or linked nucleosides of the invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

The dsRNAs packaged in the association complexes described herein can include one or more modified nucleosides, e.g., a 2′-O-methyl, 2′-O-ethyl, 2′-O-propyl, 2′-O-allyl, 2′-O-aminoalkyl or 2′-deoxy-2′-fluoro group in the nucleosides. Such modifications confer enhanced hybridization properties to the oligonucleotide. Further, oligonucleotides containing phosphorothioate backbones have enhanced nuclease stability. Thus, functionalized, linked nucleosides can be augmented to include either or both a phosphorothioate backbone or a 2′-O-methyl, 2′-O-ethyl, 2′-O-propyl, 2′-O-aminoalkyl, 2′-O-allyl or 2′-deoxy-2′-fluoro group. A summary listing of some of the oligonucleotide modifications known in the art is found at, for example, PCT Publication WO 200370918.

In some embodiments, functionalized nucleoside sequences possessing an amino group at the 5′-terminus are prepared using a DNA synthesizer, and then reacted with an active ester derivative of a selected ligand. Active ester derivatives are well known to those skilled in the art. Representative active esters include N-hydrosuccinimide esters, tetrafluorophenolic esters, pentafluorophenolic esters and pentachlorophenolic esters. The reaction of the amino group and the active ester produces an oligonucleotide in which the selected ligand is attached to the 5′-position through a linking group. The amino group at the 5′-terminus can be prepared utilizing a 5′-Amino-Modifier C6 reagent. In one embodiment, ligand molecules may be conjugated to oligonucleotides at the 5′-position by the use of a ligand-nucleoside phosphoramidite wherein the ligand is linked to the 5′-hydroxy group directly or indirectly via a linker. Such ligand-nucleoside phosphoramidites are typically used at the end of an automated synthesis procedure to provide a ligand-conjugated oligonucleotide bearing the ligand at the 5′-terminus.

Examples of modified internucleoside linkages or backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free-acid forms are also included.

Representative United States patents relating to the preparation of the above phosphorus-atom-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248, each of which is herein incorporated by reference.

Examples of modified internucleoside linkages or backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents relating to the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In certain instances, an oligonucleotide included in an association complex, such as a liposome, may be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N. Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of oligonucleotides bearing an aminolinker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate.

The modifications described above are appropriate for use with an oligonucleotide agent as described herein.

### Fusogenic Lipids

The term “fusogenic” refers to the ability of a lipid or other drug delivery system to fuse with membranes of a cell. The membranes can be either the plasma membrane or membranes surrounding organelles, e.g., endosome, nucleus, etc. Examples of suitable fusogenic lipids include, but are not limited to dioleoylphosphatidylethanolamine (DOPE), DODAC, DODMA, DODAP, or DLinDMA. In some embodiments, the association complex include a small molecule such as an imidazole moiety conjugated to a lipid, for example, for endosomal release.

### PEG or PEG-Lipids

In addition to cationic and fusogenic lipids, the association complexes include a bilayer stabilizing component (BSC) such as an ATTA-lipid or a PEG-lipid. Exemplary lipids are as follows: PEG coupled to dialkyloxypropyls (PEG-DAA) as described in, e.g., WO 05/026372, PEG coupled to diacylglycerol (PEG-DAG) as described in, e.g., U.S. Patent Publication Nos. 20030077829 and 2005008689), PEG coupled to phosphatidylethanolamine (PE) (PEG-PE), or PEG conjugated to ceramides, or a mixture thereof (see, U.S. Pat. No. 5,885,613). In a preferred embodiment, the association includes a PEG-lipid below.

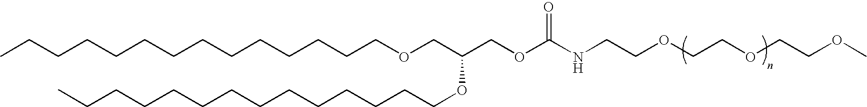

In one preferred embodiment, the BSC is a conjugated lipid that inhibits aggregation of the SPLPs. Suitable conjugated lipids include, but are not limited to PEG-lipid conjugates, ATTA-lipid conjugates, cationic-polymer-lipid conjugates (CPLs) or mixtures thereof. In one preferred embodiment, the SPLPs comprise either a PEG-lipid conjugate or an ATTA-lipid conjugate together with a CPL.

PEG is a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). In addition, monomethoxypolyethyleneglycol-acetic acid (MePEG-CH.sub.2COOH), is particularly useful for preparing the PEG-lipid conjugates including, e.g., PEG-DAA conjugates.

In a preferred embodiment, the PEG has an average molecular weight of from about 550 daltons to about 10,000 daltons, more preferably of about 750 daltons to about 5,000 daltons, more preferably of about 1,000 daltons to about 5,000 daltons, more preferably of about 1,500 daltons to about 3,000 daltons and, even more preferably, of about 2,000 daltons, or about 750 daltons. The PEG can be optionally substituted by an alkyl, alkoxy, acyl or aryl group. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH.sub.2CH.sub.2C(O)—), succinamidyl (—NHC(O)CH.sub.2CH.sub.2C(O—)NH—), ether, disulphide, etc. as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to the lipid. Suitable ester containing linker moieties include, e.g., carbonate (—OC(O)O—), succinoyl, phosphate esters (—O—(O)POH—O—), sulfonate esters, and combinations thereof.

### Targeting Agents

In some embodiments, the association complex includes a targeting agent. For example, a targeting agent can be included in the surface of the association complex (e.g., liposome) to help direct the association complex to a targeted area of the body. Examples of targeting agents are galactose, mannose, and folate. Other examples of targeting agents include small molecule receptors, peptides and antibodies. In some embodiments, the targeting agent is conjugated to the therapeutic moiety such as oligonucleotide agent. In some embodiments, the targeting moiety is attached directly to a lipid component of an association complex. In some embodiments, the targeting moiety is attached directly to the lipid component via PEG preferably with PEG of average molecular weight 2000 amu. In some embodiments, the targeting agent is unconjugated, for example on the surface of the association complex.

### Structural Components

In some embodiments, the association complex includes one or more components that improves the structure of the complex (e.g., liposome). In some embodiments, a therapeutic agents such as dsRNA can be attached (e.g., conjugated) to a lipophilic compound such as cholesterol, thereby providing a lipophilic anchor to the dsRNA. In some embodiments conjugation of dsRNA to a lipophilic moiety such as cholesterol can improve the encapsulation efficiency of the association complex.

### Properties of Association Complexes

Association complexes such as liposomes are generally particles with hydrodynamic diameter ranging from about 25 nm to 500 nm. In some preferred embodiments, the association complexes are less than 500 nm, e.g., from about 25 to about 400 nm, e.g., from about 25 nm to about 300 nm, preferably about 120 nm or less.

In some embodiments, the weight ratio of total excipients within the association complex to RNA is less than about 20:1, for example about 15:1. In some preferred embodiments, the weight ratio is less than 10:1, for example about 7.5:1.

In some embodiments the association complex has a pKₐ such that the association complex is protonated under endozomal conditions (e.g., facilitating the rupture of the complex), but is not protonated under physiological conditions.

In some embodiments, the association complex provides improved in vivo delivery of an oligonucleotide such as dsRNA. In vivo delivery of an oligonucleotide can be measured, using a gene silencing assay, for example an assay measuring the silencing of Factor VII.

### In Vivo Factor VII Silencing Experiments

C57BL/6 mice received tail vein injections of saline or various lipid formulations. Lipid-formulated siRNAs are administered at varying doses in an injection volume of 10 μL/g animal body weight. Twenty-four hours after administration, serum samples are collected by retroorbital bleed. Serum Factor VII concentrations are determined using a chromogenic diagnostic kit (Coaset Factor VII Assay Kit, DiaPharma) according to manufacturer protocols.

### Methods of Making Association Complexes

In some embodiments, an association complex is made by contacting a therapeutic agent such as an oligonucleotide with a lipid in the presence of solvent and a buffer. In some embodiments, a plurality of lipids are included in the solvent, for example, one or more of a cationic lipid (e.g., a cyclic lipid as described herein), a PEG-lipid, a targeting lipid or a fusogenic lipid.

In some embodiments, the buffer is of a strength sufficient to protonate substantially all amines of an amine containing lipid such as lipid described herein, e.g., a cyclic lipid as described herein.

In some embodiments, the buffer is an acetate buffer, such as sodium acetate (pH of about 5). In some embodiments, the buffer is present in solution at a concentration of from about 100 mM and about 300 mM.

In some embodiments, the solvent is ethanol. For example, in some embodiments, the mixture includes at least about 90% ethanol, or 100% ethanol.

In some embodiments, the method includes extruding the mixture to provide association complexes having particles of a size with hydrodynamic diameter less than about 500 nm (e.g., a size from about 25 nm to about 300 nm, for example in some preferred embodiments the particle sizes ranges from about 40-120 nm). In some embodiments, the method does not include extrusion of the mixture.

In one embodiment, a liposome is prepared by providing a solution of a lipid described herein mixed in a solution with cholesterol, PEG, ethanol, and a 25 mM acetate buffer to provide a mixture of about pH 5. The mixture is gently vortexed, and to the mixture is added sucrose. The mixture is then vortexed again until the sucrose is dissolved. To this mixture is added a solution of siRNA in acetate buffer, vortexing lightly for about 20 minutes. The mixture is then extruded (e.g., at least about 10 times, e.g., 11 times or more) through at least one filter (e.g., two 200 nm filters) at 40° C., and dialyzed against PBS at pH 7.4 for about 90 minutes at RT.

In one embodiment, an association complex such as a liposome is prepared without extruding the liposome mixture. A lipid described herein is combined with cholesterol, PEG, and siRNA in 100% ethanol, water, and an acetate buffer having a concentration from about 100 mM to about 300 mM (pH of about 5). The combination is rapidly mixed in 90% ethanol. Upon completion, the mixture is dialyzed (or treated with ultrafiltration) against an acetate buffer having a concentration from about 100 mM to about 300 mM (pH of about 5) to remove ethanol, and then dialyzed (or treated with ultrafiltration) against PBS to change buffer conditions.

Association complexes can, be formed in the absence of a therapeutic agent such as single or double stranded nucleic acid, and then upon formation be treated with one or more therapeutically active single or double stranded nucleic acid moieties to provide a loaded association complex, i.e., an association complex that is loaded with the therapeutically active nucleic acids. The nucleic acid can be entrapped within the association complex, adsorbed to the surface of the association complex or both. For example, methods of forming association complexes such as liposomes above can be used to form association complexes free of a therapeutic agent, such as a nucleic acid, for example a single or double stranded RNA such as siRNA. Upon formation of the association complex, the complex can then be treated with the therapeutic agent such as siRNA to provide a loaded association complex.

In one embodiment, a mixture including cationic lipid such as a cationic lipid, and a PEG-lipid, for example the PEG-lipid below,

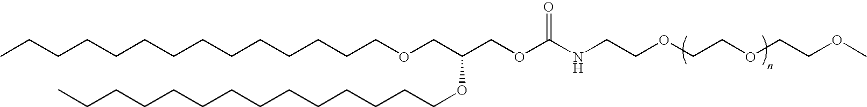

are provided in ethanol (e.g., 100% ethanol) and combined with an aqueous buffer such as aqueous NaOAc, to provide unloaded association complexes. The association complexes are then optionally extruded, providing a more uniform size distribution of the association complexes. The association complexes are then treated with the therapeutic agent such as siRNA in ethanol (e.g., 35% ethanol) to thereby provide a loaded association complex. In some embodiments, the association complex is then treated with a process that removes the ethanol, such as dialysis.

### Characterization of Association Complexes

Association complexes prepared by any of the methods above are characterized in a similar manner. Association complexes are first characterized by visual inspection. In general, preferred association complexes are whitish translucent solutions free from aggregates or sediment. Particle size and particle size distribution of lipid-nanoparticles are measured by dynamic light scattering using a Malvern Zetasizer Nano ZS (Malvern, USA). Preferred particles are 20-300 nm, more preferably, 40-100 nm in size. In some preferred embodiments, the particle size distribution is unimodal. The total siRNA concentration in the formulation, as well as the entrapped fraction, is estimated using a dye exclusion assay. A sample of the formulated siRNA is incubated with the RNA-binding dye Ribogreen (Molecular Probes) in the presence or absence of a formulation disrupting surfactant, 0.5% Triton-X100. The total siRNA in the formulation is determined by the signal from the sample containing the surfactant, relative to a standard curve. The entrapped fraction is determined by subtracting the “free” siRNA content (as measured by the signal in the absence of surfactant) from the total siRNA content. Percent entrapped siRNA is typically >85%.

### Methods of Using Association Complexes and Compositions Including the Same Pharmaceutical Compositions Comprising Oligonucleotide Agents

An oligonucleotide agent assembled in an association complex can be administered, e.g., to a cell or to a human, in a single-stranded or double-stranded configuration. An oligonucleotide agent that is in a double-stranded configuration is bound to a substantially complementary oligonucleotide strand. Delivery of an oligonucleotide agent in a double stranded configuration may confer certain advantages on the oligonucleotide agent, such as an increased resistance to nucleases.

In one embodiment, the invention provides pharmaceutical compositions including an oligonucleotide agent packaged in an association complex, such as a liposome, as described herein, and a pharmaceutically acceptable carrier. The pharmaceutical composition comprising the packaged oligonucleotide agent is useful for treating a disease or disorder associated with the expression or activity of a target gene, such as a pathological process which can be mediated by down regulating gene expression. Such pharmaceutical compositions are formulated based on the mode of delivery. One example is compositions that are formulated for delivery to a specific organ/tissue, such as the liver, via parenteral delivery.

The pharmaceutical compositions featured in the invention are administered in dosages sufficient to inhibit expression of a target gene.

In general, a suitable dose of a packaged oligonucleotide agent will be such that the oligonucleotide agent delivered is in the range of 0.01 to 5.0 milligrams per kilogram body weight of the recipient per day, generally in the range of 1 microgram to 1 mg per kilogram body weight per day. The pharmaceutical composition may be administered once daily, or the oligonucleotide agent may be administered as two, three, or more sub-doses at appropriate intervals throughout the day or even using continuous infusion or delivery through a controlled release formulation. In that case, the oligonucleotide agent contained in each sub-dose must be correspondingly smaller in order to achieve the total daily dosage. The dosage unit can also be compounded for delivery over several days, e.g., using a conventional sustained release formulation which provides sustained release of the packaged oligonucleotide agent over a several day period. Sustained release formulations are well known in the art.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual oligonucleotide agents packaged in the association complexes can be made using conventional methodologies or on the basis of in vivo testing using an appropriate animal model, as described elsewhere herein.

Advances in mouse genetics have generated a number of mouse models for the study of various human diseases. Such models are used for in vivo testing of oligonucleotide agents packaged in lipophilic compositions, as well as for determining a therapeutically effective dose.

Any method can be used to administer an oligonucleotide agent packaged in an association complex, such as a liposome, to a mammal. For example, administration can be direct; oral; or parenteral (e.g., by subcutaneous, intraventricular, intramuscular, or intraperitoneal injection, or by intravenous drip). Administration can be rapid (e.g., by injection), or can occur over a period of time (e.g., by slow infusion or administration of slow release formulations).

An oligonucleotide agent packaged in an association complex can be formulated into compositions such as sterile and non-sterile aqueous solutions, non-aqueous solutions in common solvents such as alcohols, or solutions in liquid or solid oil bases. Such solutions also can contain buffers, diluents, and other suitable additives. For parenteral, intrathecal, or intraventricular administration, an oligonucleotide agent can be formulated into compositions such as sterile aqueous solutions, which also can contain buffers, diluents, and other suitable additives (e.g., penetration enhancers, carrier compounds, and other pharmaceutically acceptable carriers).

The oligonucleotide agents packaged in an association complex can be formulated in a pharmaceutically acceptable carrier or diluent. A “pharmaceutically acceptable carrier” (also referred to herein as an “excipient”) is a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

### EXAMPLES

### Example 1

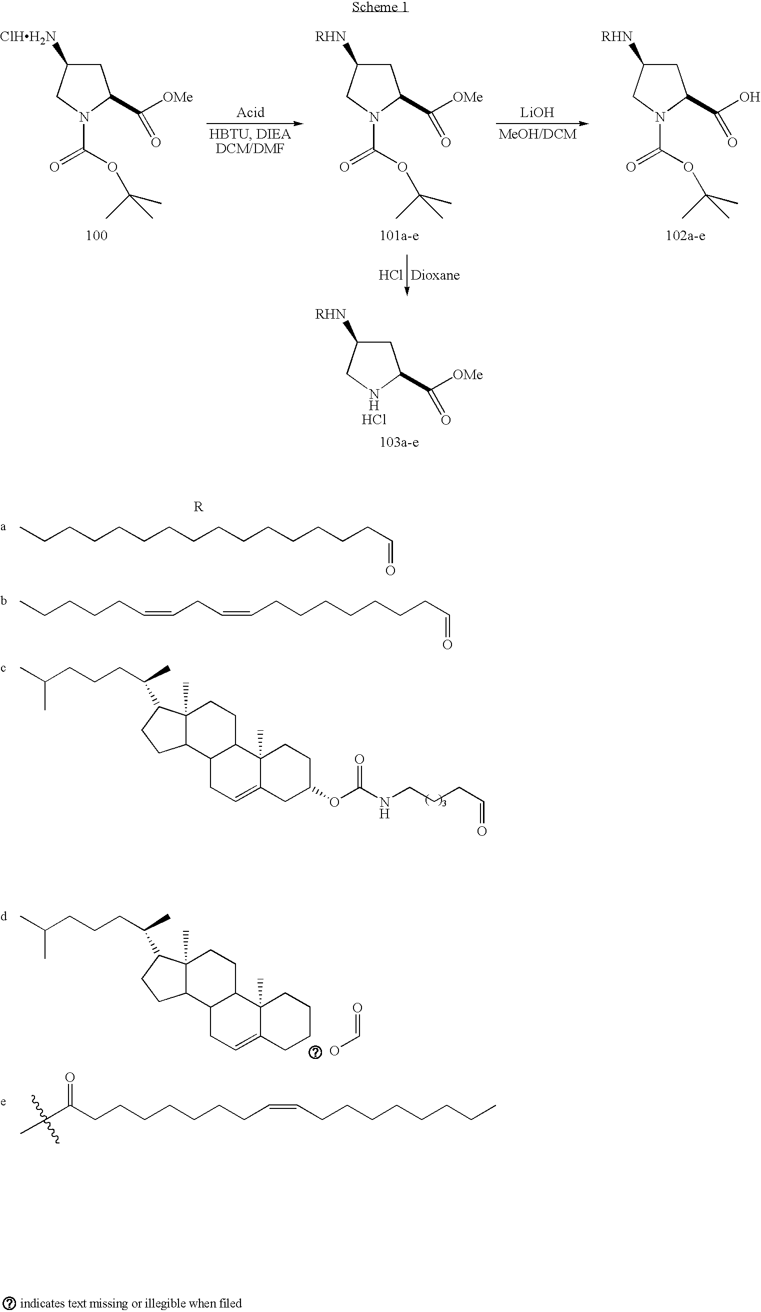

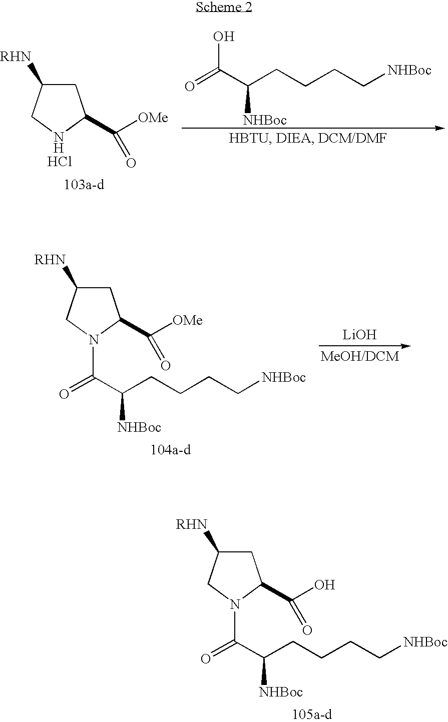

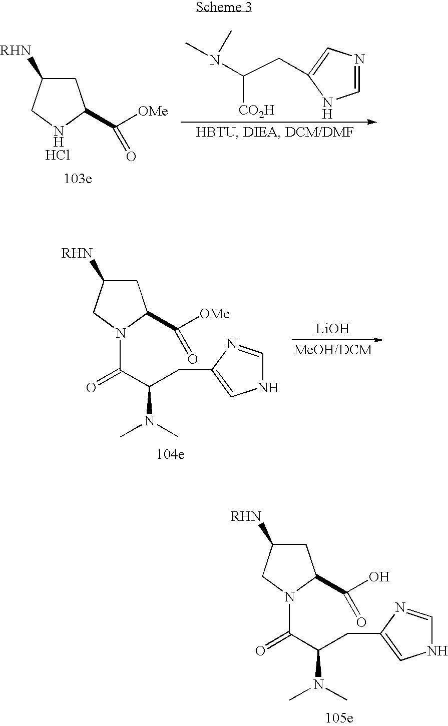

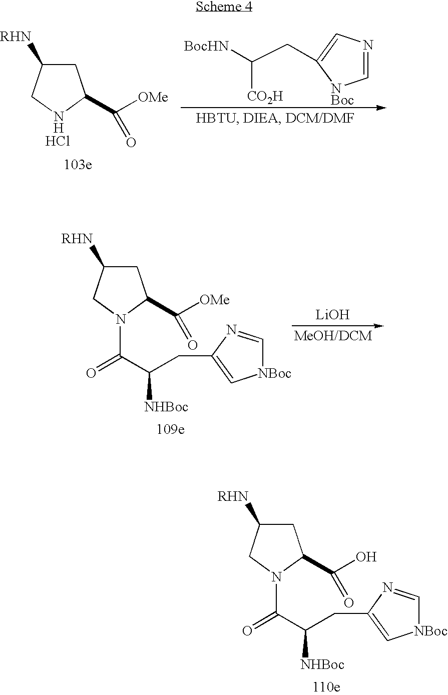

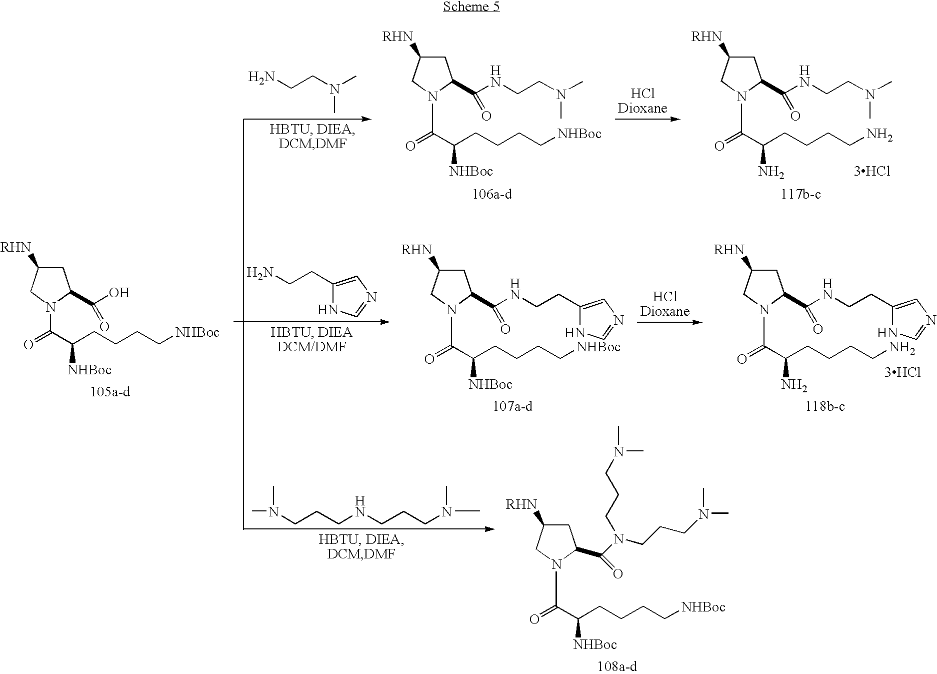

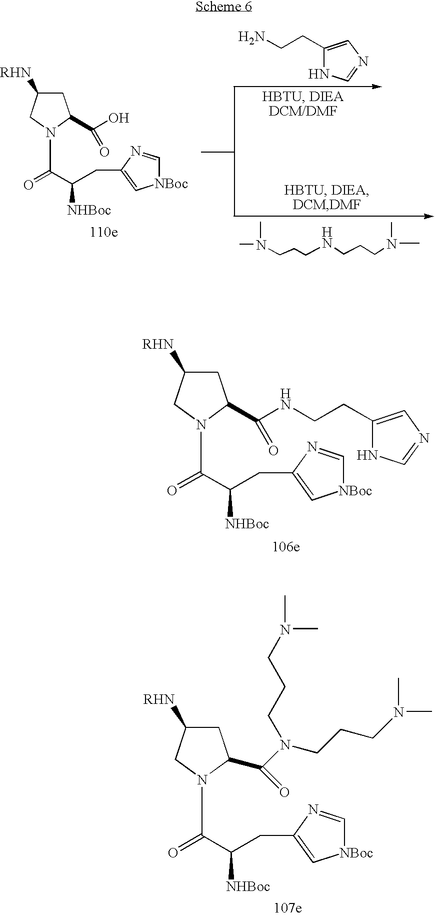

Preparation of 106a: Compound 105a (1.13 g, 1.62 mmol) and HBTU (0.738 g, 1.2 eq.) were taken together in a mixture of DCM/DMF (2:1). To that DIEA (0.732 ml, 3 eq.) was added, stirred the mixture for 5 minutes. N,N-Dimethyl ethylene diamine (0.266 mL, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 106a (1.08 g, 84%). ¹H NMR (CDCl₃, 400 MHz) δ=MS Cal. for C₄₃H₈₂N₆O₇ 794.62 Found: 795.6 (M+H)

Preparation of 107a: Compound 105a (1.04 g, 1.49 mmol) and HBTU (0.680 g, 1.2 eq.) were taken together in a mixture of DCM/DMF (2:1). To that DIEA (0.80 ml, 3 eq.) was added, stirred the mixture for 5 minutes. Histamine (0.250 g, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 107a (1.0 g, 85%). ¹H NMR (CDCl₃, 400 MHz) δ=MS Cal. for C₄₄H₇₉N₇O₇ 817.60 Found: 818.6 (M+H).

Preparation of 108a: Compound 105a (1.17 g, 1.67 mmol) and HBTU (0.764 g, 1.2 eq.) were taken together in a mixture of DCM/DMF (2:1). To that DIEA (0.876 ml, 3 eq.) was added, stirred the mixture for 5 minutes. N,N,N′,N′-Tetramethyliminobispropylamine (0.561 mL, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 108a (1.21 g, 81%). ¹H NMR (CDCl₃, 400 MHz) δ=MS Cal. for C₄₉H₉₅N₇O₇ 893.73 Found: 894.7 (M+H).

Preparation of 106b: Compound 105b (1.10 g, 1.53 mmol) and HBTU (0.696 g, 1.2 eq.) were taken together in a mixture of DCM/DMF (2:1). To that DIEA (0.800 ml, 3 eq.) was added, stirred the mixture for 5 minutes. N,N-Dimethyl ethylene diamine (0.250 mL, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 106b (0.99 g, 76%). ¹H NMR (CDCl₃, 400 MHz) δ=MS Cal. for C₄₃H₇₈N₆O₇ 790.59 Found: 791.6 (M+H)

Preparation of 107b: Compound 105b (1.19 g, 1.65 mmol) and HBTU (0.751 g, 1.2 eq.) were taken together in a mixture of DCM/DMF (2:1). To that DIEA (0.86 ml, 3 eq.) was added, stirred the mixture for 5 minutes. Histamine (0.276 g, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 107b (1.15 g, 86%). ¹H NMR (CDCl₃, 400 MHz) δ=MS Cal. for C₄₄H₇₅N₇O₇ 813.57 Found: 814.5 (M+H).

Preparation of 108b: Compound 105b (1.19 g, 1.65 mmol) and HBTU (0.751 g, 1.2 eq.) were taken together in a mixture of DCM/DMF (2:1). To that DIEA (0.86 ml, 3 eq.) was added, stirred the mixture for 5 minutes. N,N,N′,N′-Tetramethyliminobispropylamine (0.461 mL, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 108b (1.15 g, 78%). ¹H NMR (CDCl₃, 400 MHz) δ=MS Cal. for C₄₉H₉₁N₇O₇ 889.70 Found: 890.7 (M+H).

Preparation of 106c: Compound 105c (1.00 g, 1.02 mmol) and HBTU (0.462 g, 1.2 eq.) were taken together in a mixture of DCM/DMF (2:1). To that DIEA (0.529 ml, 3 eq.) was added, stirred the mixture for 5 minutes. N,N-Dimethyl ethylene diamine (0.166 mL, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 106c (0.84 g, 76%). ¹H NMR (CDCl₃, 400 MHz) δ=MS Cal. for C₅₉H₁₀₃N₇O₉ 1053.78 Found: 1054.8 (M+H)

Preparation of 107c: Compound 105c (1.19 g, 1.02 mmol) and HBTU (0.462 g, 1.2 eq.) were taken together in a mixture of DCM/DMF (2:1). To that DIEA (0.530 ml, 3 eq.) was added, stirred the mixture for 5 minutes. Histamine (0.170 g, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 107c (0.88 g, 81%). ¹H NMR (CDCl₃, 400 MHz) δ=MS Cal. for C₆₀H₁₀₀N₈O₉ 1076.76 Found: 1077.7 (M+H).

Preparation of 108c: Compound 105c (1.00 g, 1.02 mmol) and HBTU (0.462 g, 1.2 eq.) were taken together in a mixture of DCM/DMF (2:1). To that DIEA (0.530 ml, 3 eq.) was added, stirred the mixture for 5 minutes. N,N,N′,N′-Tetramethyliminobispropylamine (0.34 mL, 1.5 eq.) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 108c (0.85 g, 72%). MS Cal. for C₆₅H₁₁₆N₈O₉ 1152.89 Found: 1153.9 (M+H).

Preparation of 106e: Compound 110e (1.1 g, 1.5 mmol) and HBTU (0.57 g, 1 eq.) were taken together in a mixture of DCM/DMF (2:1). To that DIEA (0.57 g, 3 eq.) was added, stirred the mixture for 5 minutes. Histamine (0.170 g, 1.5 mmol) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 106e (0.86 g, 78%). MS Cal. for C₄₄H₇₂N₈O₇ 825.09 Found: 826.1 (M+H).

Preparation of 107e: Compound 110e (1.1 g, 1.5 mmol) and HBTU (0.57 g, 1 eq.) were taken together in a mixture of DCM/DMF (2:1). To that DIEA (0.57 g, 3 eq.) was added, stirred the mixture for 5 minutes. N,N,N′,N′-Tetramethyliminobispropylamine (0.28 g, 1.5 mmol) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 107e (0.86 g, 72%). MS Cal. for C₄₉H₈₈N₈O₇ 901.27 Found: 902.3 (M+H).

Preparation of 117b: Compound 106b (0.97 g, 1.22 mmol) was taken in RB flask to that 20 mL of HCl solution in dioxane (4M) was added and the mixture stirred overnight. Volatiles were removed under reduced pressure and the residue co-evaporated with ethanol three times to get the required product (800 mg, 93%). MS Cal. for C₃₃H₆₆N₆O₃ 594.52. Found 595.5 (M+H).

Preparation of 117c: Compound 106c (0.82 g, 0.78 mmol) was taken in RB flask to that 20 mL of HCl solution in dioxane (4M) was added and the mixture stirred overnight. Volatiles were removed under reduced pressure and the residue co-evaporated with ethanol three times to get the required product (635 mg, 86%). MS Cal. for C₄₉H₈₇N₇O₅ 853.68 Found: 854.7 (M+H).

Preparation of 118b: Compound 107b (1.13 g, 1.39 mmol) was taken in RB flask to that 20 mL of HCl solution in dioxane (4M) was added and the mixture stirred overnight. Volatiles were removed under reduced pressure and the residue co-evaporated with ethanol three times to get the required product (920 mg, 92%). MS Cal. for C₃₄H₆₃N₇O₃ 617.50 Found: 618.5 (M+H).

Preparation of 118c: Compound 107c (0.86 g, 0.80 mmol) was taken in RB flask to that 20 mL of HCl solution in dioxane (4M) was added and the mixture stirred overnight. Volatiles were removed under reduced pressure and the residue co-evaporated with ethanol three times to get the required product (730 mg, 93.5%). MS Cal. for C₅₀H₈₄N₈O₅ 876.66 Found: 877.6 (M+H).

### Example 2

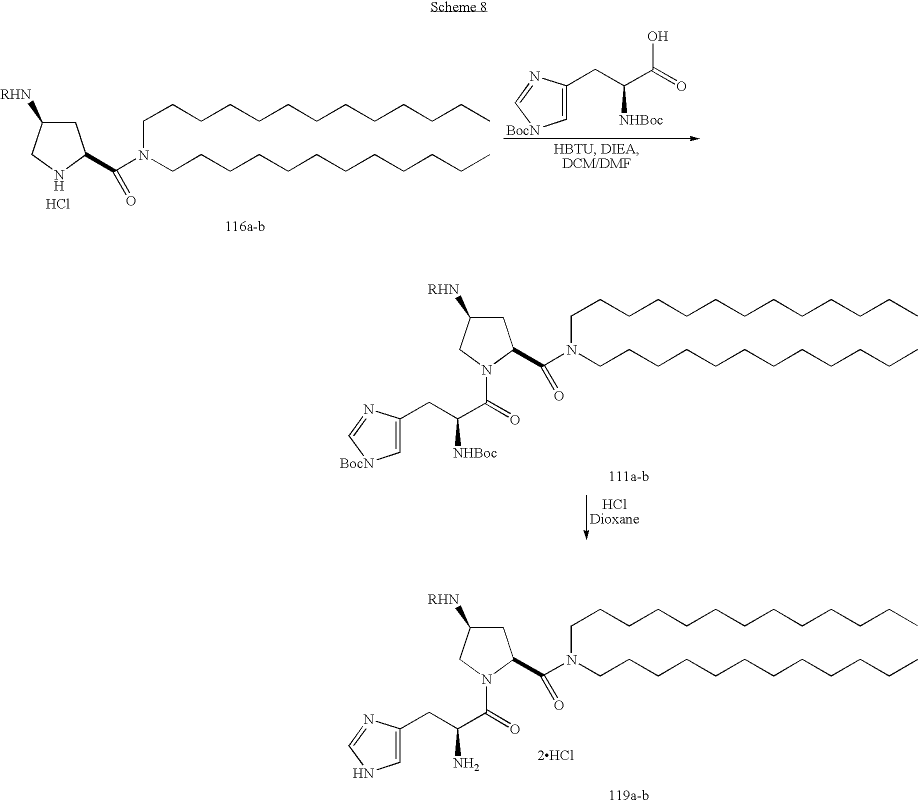

Preparation of 111a: Compound 116a (1.10 g, 1.48 mmol) and (Boc)₂ histidine (0.785 g, 1.81 mmol) were taken together in a mixture of DCM/DMF (2:1). To that HBTU (0.688 g, 1.81 mmol) was added, followed by DIEA (0.787 mL, 3 eq.). The mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (gradient elution 30-80% ethyl acetate/hexane) to get 111a (1.18 g, 77%). ¹H NMR (CDCl₃, 400 MHz) δ=MS Cal. for C₆₃H₁₁₆N₆O₇ 1068.89 Found: 1069.9 (M+H)

Preparation of 111b: Compound 116b (1.22 g, 1.595 mmol) and (Boc)₂ histidine (0.829 g, 1.2 eq.) were taken together in a mixture of DCM/DMF (2:1, 25 mL). To that HBTU (0.726 g, 1.2 eq.) was added, followed by DIEA (0.832 mL, 3 eq.). The mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (gradient elution 20-80% ethyl acetate/hexane) to get 111b (1.20 g, 71%). ¹H NMR (CDCl₃, 400 MHz) δ=MS Cal. for C₆₃H₁₁₂N₆O₇ 1064.88 Found: 1065.8 (M+H).

Preparation of 119a: Compound 111a (1.16 g, 1.08 mmol) was taken in RB flask to that 20 mL of HCl solution in dioxane (4M) was added and the mixture stirred overnight. Volatiles were removed under reduced pressure and the residue co-evaporated with ethanol three times to get the required product (680 mg, 66%). MS Cal. for C₅₃H₁₀₀N₆O₃ 868.79 Found: 869.70 (M+H).

Preparation of 119b: Compound 111b (1.18 g, 1.10 mmol) was taken in RB flask to that 20 mL of HCl solution in dioxane (4M) was added and the mixture stirred overnight. Volatiles were removed under reduced pressure and the residue co-evaporated with ethanol three times to get the required product (900 mg, 87%). MS Cal. for C₅₃H₉₆N₆O₃ 864.75 Found: 865.7 (M+H).

### Example 3

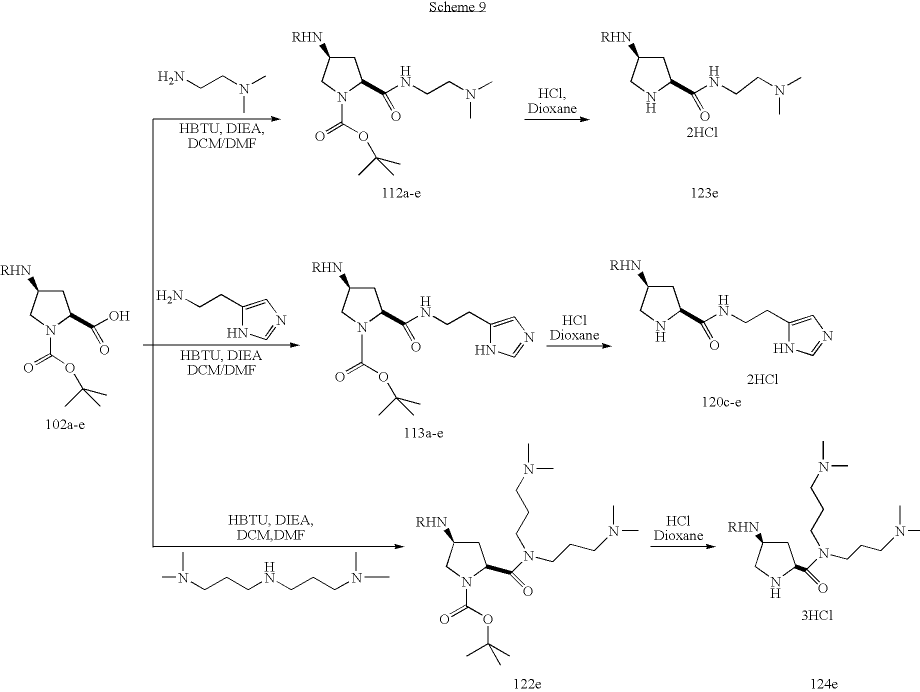

Preparation of 112a: Compound 102a (1.00 g, 2.01 mmol) and HBTU (0.837 g, 1.1 eq) were taken in a mixture of DCM/DMF (2:1). To that DIEA (1.04 mL, 3 eq.) was added, the mixture stirred for 5 minutes. N,N-Dimethyl ethylene diamine (0.265 mL, 1.5 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 112a (0.890 g, 78%). ¹H NMR (CDCl₃, 400 MHz) δ=MS Cal. for C₃₂H₆₂N₄O₄ 566.48 Found: 567.5 (M+H)

Preparation of 112b: Compound 102b (1.05 g, 2.13 mmol) and HBTU (0.852 g, 1.1 eq) were taken in a mixture of DCM/DMF (2:1). To that DIEA (1.10 mL, 3 eq.) was added, the mixture stirred for 5 minutes. N,N-Dimethyl ethylene diamine (0.333 mL, 1.5 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 112b (0.950 g, 76%). ¹H NMR (CDCl₃, 400 MHz) δ=MS Cal. for C₃₂H₅₈N₄O₄ 562.45 Found: 563.4 (M+H)

Preparation of 112c: Compound 102c (0.830 g, 1.098 mmol) and HBTU (0.500 g, 1.2 eq) were taken in a mixture of DCM/DMF (2:1). To that DIEA (0.572 mL, 3 eq.) was added, the mixture stirred for 5 minutes. N,N-Dimethyl ethylene diamine (0.179 mL, 1.5 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 112c (0.730 g, 80.5%). ¹H NMR (CDCl₃, 400 MHz) δ=MS Cal. for C₄₈H₈₃N₅O₆ 825.63 Found: 826.6 (M+H)

Preparation of 112d: Compound 102d (1.00 g, 1.55 mmol) and HBTU (0.648 g, 1.1 eq) were taken in a mixture of DCM/DMF (2:1). To that DIEA (0.81 mL, 3 eq.) was added, the mixture stirred for 5 minutes. N,N-Dimethyl ethylene diamine (0.203 mL, 1.5 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 112d (0.96 g, 87%). ¹H NMR (CDCl₃, 400 MHz) δ=MS Cal. for C₄₂H₇₂N₄O₅ 712.55 Found: 713.04 (M+H)

Preparation of 113a: Compound 102a (1.00 g, 2.01 mmol) and HBTU (0.837 g, 1.1 eq) were taken in a mixture of DCM/DMF (2:1). To that DIEA (1.04 mL, 3 eq.) was added, the mixture stirred for 5 minutes. Histamine (0.309 g, 1.3 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 113a (1.04 g, 78%). ¹H NMR (CDCl₃, 400 MHz) δ=MS Cal. for C₃₃H₅₉N₅O₄ 589.46 Found: 590.5 (M+H).

Preparation of 113b: Compound 102b (1.03 g, 2.09 mmol) and HBTU (0.846 g, 1.1 eq) were taken in a mixture of DCM/DMF (2:1). To that DIEA (1.058 mL, 3 eq.) was added, the mixture stirred for 5 minutes. Histamine (0.297 g, 1.3 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 113b (1.08 g, 88%). ¹H NMR (CDCl₃, 400 MHz) δ=MS Cal. for C₃₃H₅₅N₅O₄ 585.43 Found: 586.4 (M+H).

Preparation of 113c: Compound 102c (0.91 g, 1.20 mmol) and HBTU (0.546 g, 1.2 eq) were taken in a mixture of DCM/DMF (2:1). To that DIEA (0.625 mL, 3 eq.) was added, the mixture stirred for 5 minutes. Histamine (0.207 g, 1.5 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 113c (0.64 g, 63%). ¹H NMR (CDCl₃, 400 MHz) δ=MS Cal. for C₄₉H₈₀N₆O₆ 848.61 Found: 848.6 (M+H).

Preparation of 113d: Compound 102d (1.00 g, 1.55 mmol) and HBTU (0.648 g, 1.1 eq) were taken in a mixture of DCM/DMF (2:1). To that DIEA (0.81 mL, 3 eq.) was added, the mixture stirred for 5 minutes. Histamine (0.191 g, 1.1 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 113d (0.94 g, 82%). ¹H NMR (CDCl₃, 400 MHz) δ=MS Cal. for C₄₃H₆₉N₅O₅ 735.53 Found: 736.5 (M+H)

Preparation of 120c: Compound 113c (0.620 g, 0.717 mmol) was taken in RB flask to that 20 mL of HCl solution in dioxane (4M) was added and the mixture stirred overnight. Volatiles were removed under reduced pressure and the residue co-evaporated with ethanol three times to get the required product (150 mg, 25%). MS Cal. for C₄₄H₇₂N₆O₄ 748.56 Found: 749.5 (M+H).

Preparation of 120d: Compound 113d (0.92 g, 1.25 mmol) was taken in RB flask to that 20 mL of HCl solution in dioxane (4M) was added and the mixture stirred overnight. Volatiles were removed under reduced pressure and the residue co-evaporated with ethanol three times to get the required product (700 mg, 79%). MS Cal. for C₃₈H₆₁N₅O₃ 635.48 Found: 636.4 (M+H).

### Example 4

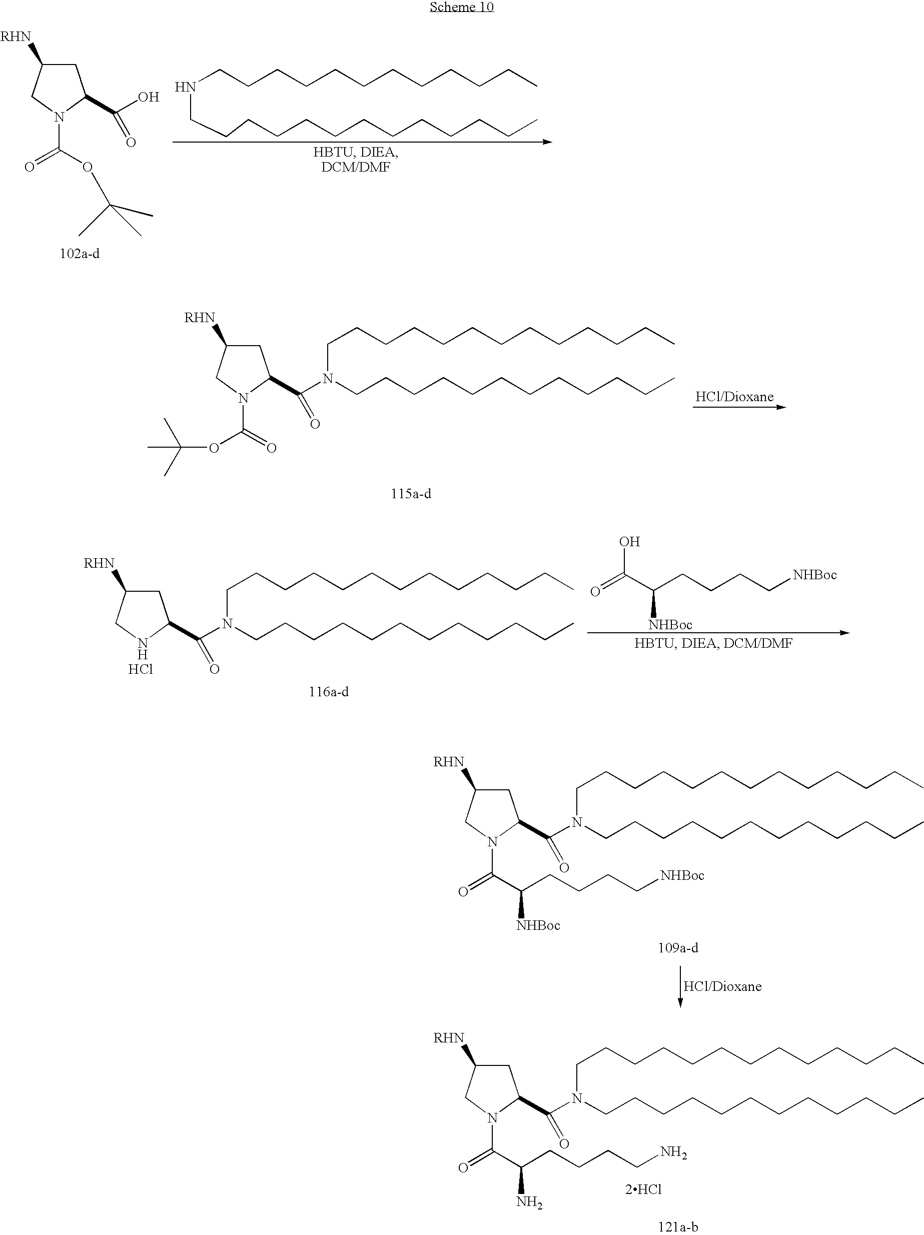

Preparation of 109a: Compound 116a (1.02 g, 1.40 mmol) and (Boc)₂ lysine (0.614 g, 1.2 eq.) were taken together in a mixture of DCM/DMF (2:1). To that HBTU (0.638 g, 1.2 eq.) was added, followed by DIEA (0.732 ml, 3 eq.). The mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (gradient elution 10-40% ethyl acetate/hexane) to get 109a (1.18 g, 84.3%). ¹H NMR (CDCl₃, 400 MHz) δ=MS Cal. for C₆₃H₁₂₁N₅O₇ 1059.93 Found: 1060.9 (M+H)

Preparation of 109b: Compound 116b (1.26 g, 1.65 mmol) and (Boc)₂ lysine (0.720 g, 1.2 eq.) were taken together in a mixture of DCM/DMF (2:1, 25 mL). To that HBTU (0.749 g, 1.2 eq.) was added, followed by DIEA (0.859 ml, 3 eq.). The mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (gradient elution 20-40% ethyl acetate/hexane) to get 109b (1.40 g, 81%). ¹H NMR (CDCl₃, 400 MHz) δ=MS Cal. for C₆₃H₁₁₇N₅O₇ 1055.90 Found: 1056.9 (M+H).

Preparation of 121a: Compound 109a (1.17 g, 1.10 mmol) was taken in RB flask to that 20 mL of HCl solution in dioxane (4M) was added and the mixture stirred overnight. Volatiles were removed under reduced pressure and the residue co-evaporated with ethanol three times to get the required product (700 mg, 69%). MS Cal. for C₅₃H₁₀₅N₅O₃ 859.82 Found: 860.8 (M+H).

Preparation of 121b: Compound 109b (1.38 g, 1.30 mmol) was taken in RB flask to that 20 mL of HCl solution in dioxane (4M) was added and the mixture stirred overnight. Volatiles were removed under reduced pressure and the residue co-evaporated with ethanol three times to get the required product (830 mg, 68%). MS Cal. for C₅₃H₁₀₁N₅O₃ 855.79 Found: 856.8 (M+H).

### Example 5

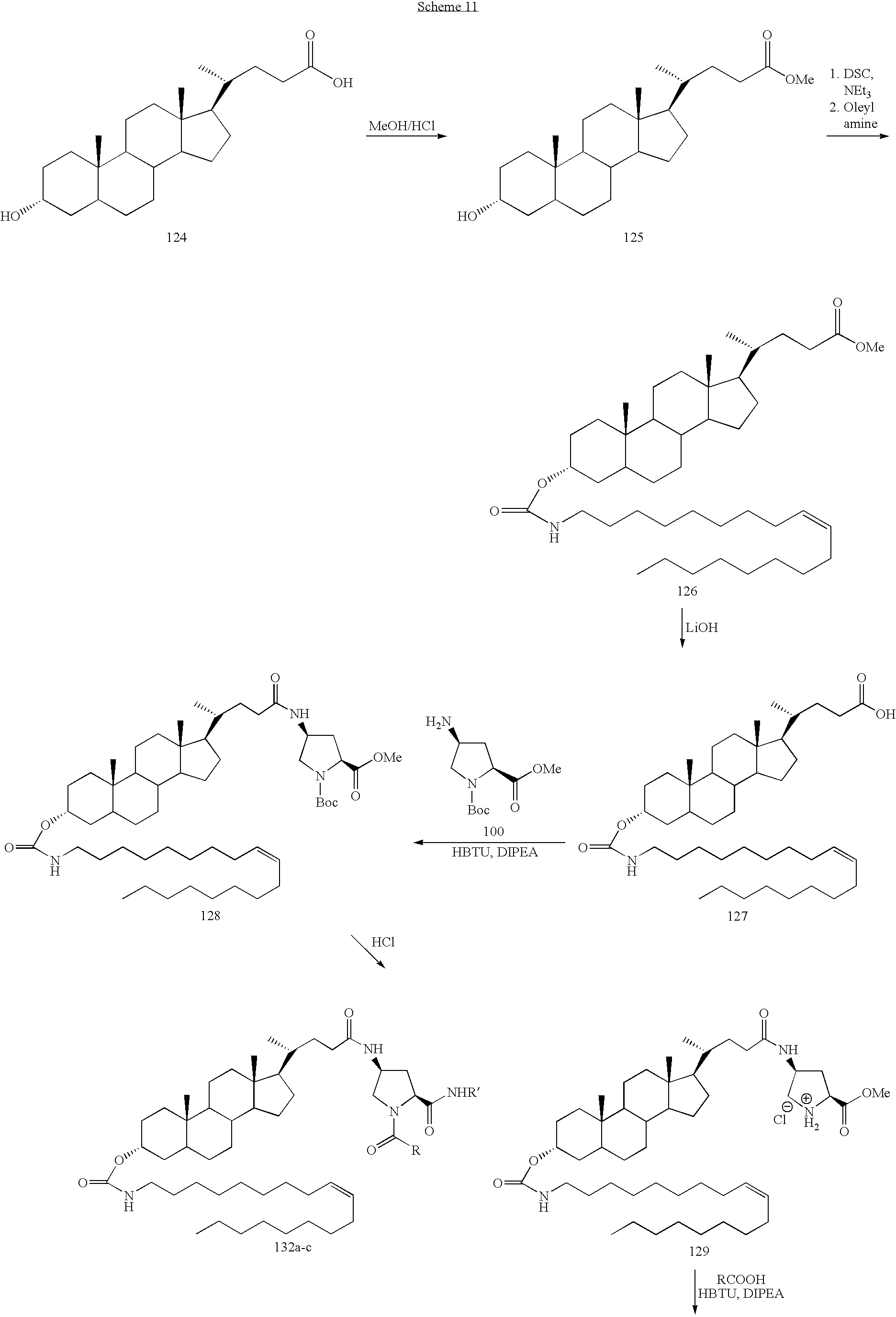

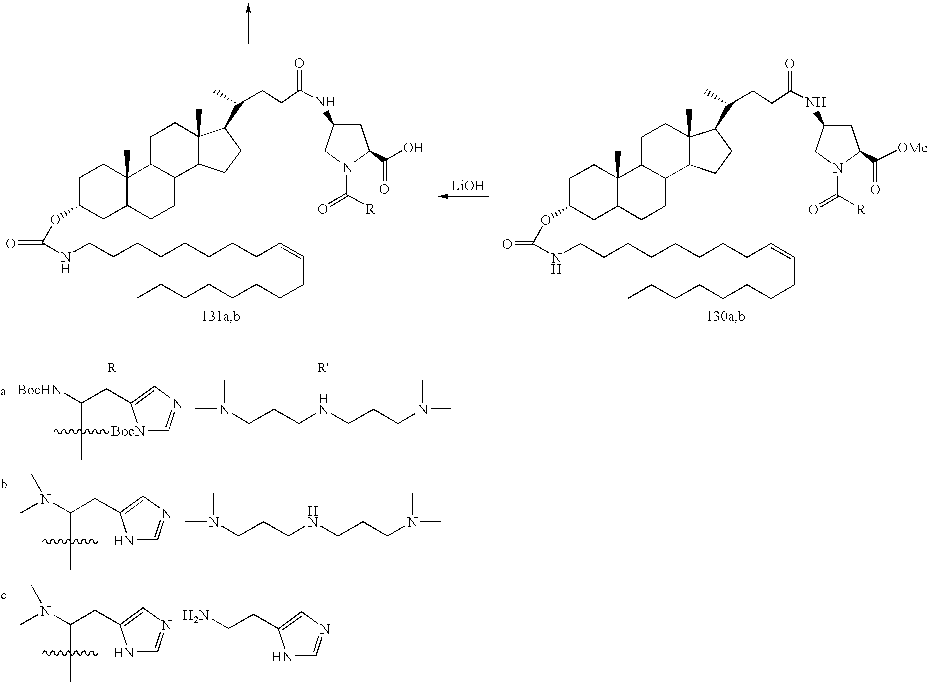

Preparation of 132a: Compound 131a (1.1 g, 1 mmol) and HBTU (0.379 g, 1 eq.) were taken together in a mixture of DCM/DMF (2:1). To that DIEA (0.38 g, 3 eq.) was added, stirred the mixture for 5 minutes. N,N,N′,N′-Tetramethyliminobispropylamine (0.187 g, 1 mmol) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue was purified by chromatography (first ethyl acetate then gradient elution 5-10% MeOH/DCM) to get 132a (0.5 g, 43%). MS Cal. for C₇₄H₁₂₉N₉O₉ 1288.87 Found: 1289.9 (M+H).

Preparation of 132b: Compound 131b (1.1 g, 1.16 mmol) and HBTU (0.455 g, 1 eq.) were taken together in a mixture of DCM/DMF (2:1). To that DIEA (0.457 g, 3 eq.) was added, stirred the mixture for 5 minutes. N,N,N′,N′-Tetramethyliminobispropylamine (0.217 g, 1 mmol) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue is purified by chromatography (first ethyl acetate then gradient elution 5-10% MeOH/DCM) to get 132a. MS Cal. for C₆₆H₁₁₇N₉O₅ 1116.69 Found: 1117.9 (M+H).

Preparation of 132c: Compound 131b (1.1 g, 1.16 mmol) and HBTU (0.455 g, 1 eq) were taken in a mixture of DCM/DMF (2:1). To that DIEA (0.457 g, 3 eq.) was added, the mixture stirred for 5 minutes. Histamine (0.129 g, 1.3 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue is purified by chromatography to get 132c. MS Cal. for C₆₁H₁₀₁N₉O₅ 1040.51 Found: 1041.5 (M+H).

### Example 6

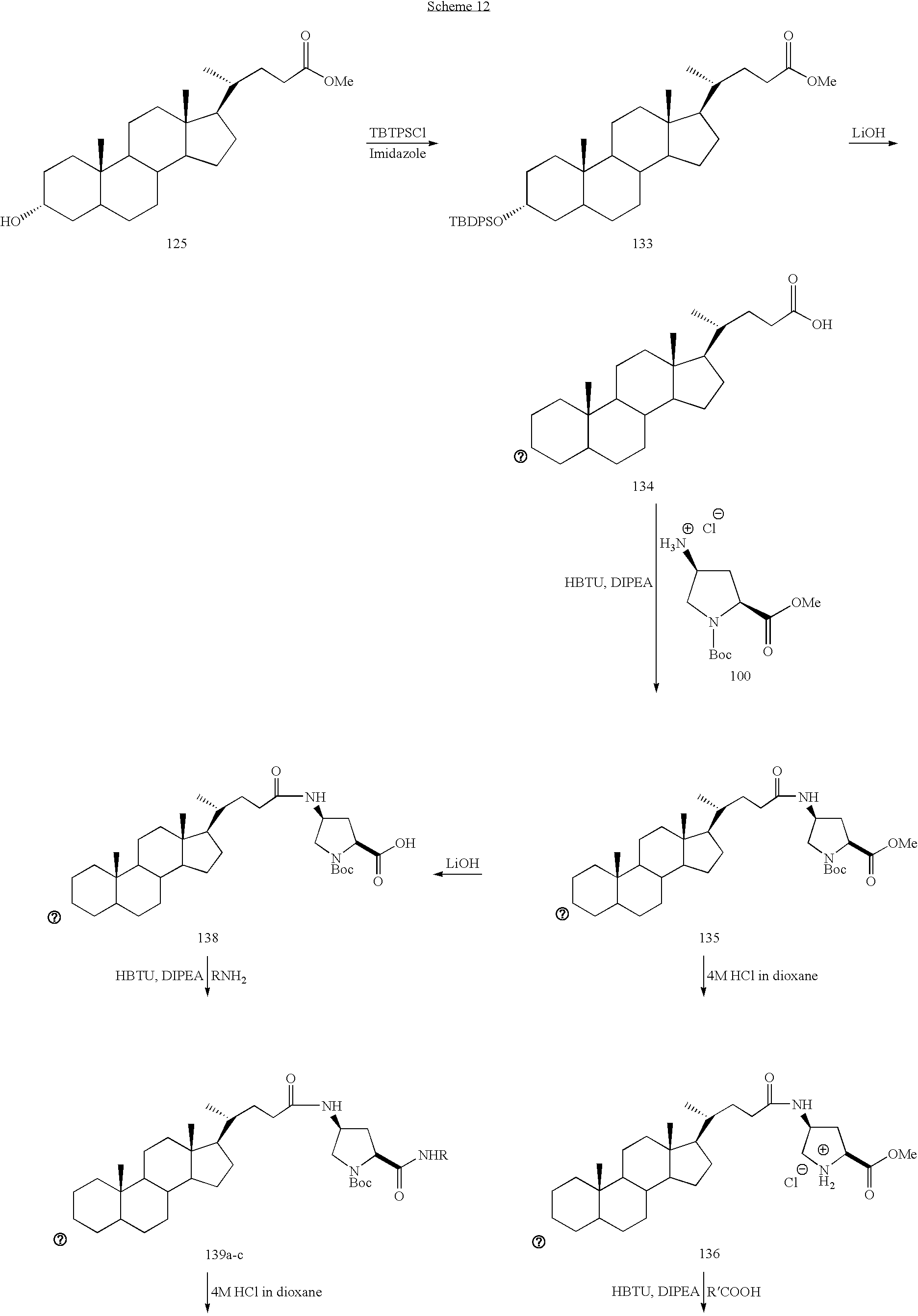

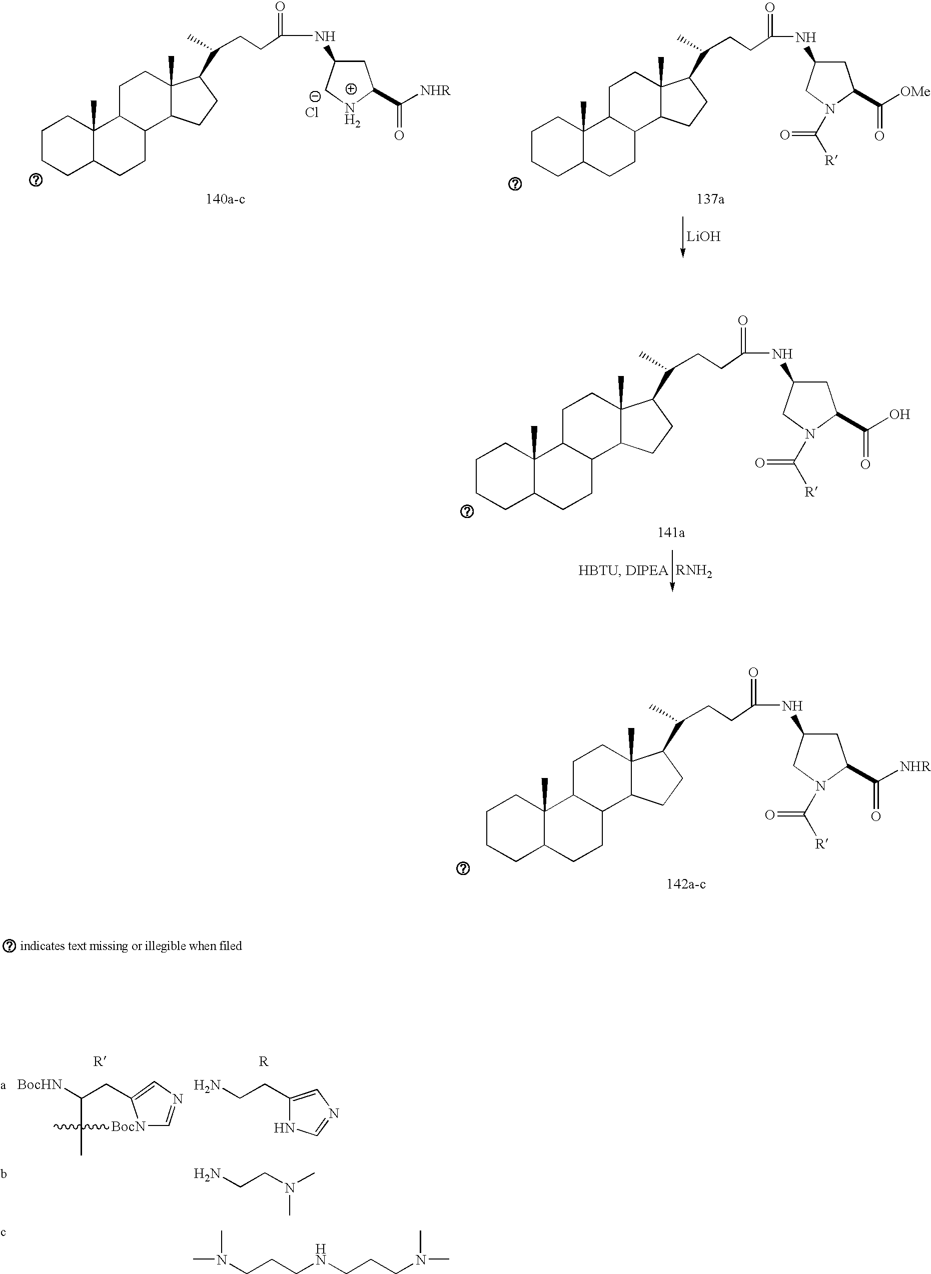

Preparation of 139a: Compound 138 (0.827 g, 1 mmol) and HBTU (0.379 g, 1 eq) were taken in a mixture of DCM/DMF (2:1). To that DIEA (0.381 g, 3 eq.) was added, the mixture stirred for 5 minutes. Histamine (0.111 g, 1 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 139a (0.856 g, 93%). MS Cal. for C₅₅H₈₁N₅O₅Si 920.35 Found: 921.5 (M+H).

Preparation of 139b: Compound 138 (0.827 g, 1 mmol) and HBTU (0.379 g, 1 eq) were taken in a mixture of DCM/DMF (2:1). To that DIEA (0.381 g, 3 eq.) was added, the mixture stirred for 5 minutes. N,N-Dimethyl ethylene diamine (0.088 g, 1 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue was purified by chromatography (first eluted with ethyl acetate followed by a gradient elution of 5-10% MeOH/DCM) to get 139b (0.760 g, 85%). MS Cal. for C₅₄H₈₄N₄O₅Si 897.35 Found: 898.3 (M+H).

Preparation of 139c: Compound 138 (0.827 g, 1 mmol) and HBTU (0.379 g, 1 eq.) were taken together in a mixture of DCM/DMF (2:1). To that DIEA (0.38 g, 3 eq.) was added, stirred the mixture for 5 minutes. N,N,N′,N′-Tetramethyliminobispropylamine (0.187 g, 1 mmol) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue is purified by chromatography to get 139c. MS Cal. for C₆₀H₉₇N₅O₅Si 996.53 Found: 997.5 (M+H).

Preparation of 140a: Compound 139a (0.856 g, 0.93 mmol) was stirred at ambient temperature with 4M hydrochloric acid in dioxane (20 mL). After 16 h, the completion of the reaction was confirmed by MS and the reaction mixture was concentrated and to the residue, ethyl acetate was added and the precipitated product was filtered, washed with hexanes and dried in the vacuum oven at 45° C. overnight. The pure hydrochloride salt 140a was isolated (0.36 g, 50%) as a white powder. MS Cal. for C₃₄H₅₅N₅O₃ 2HCl; 654.75 Found: 582.4 (M+H, free base).

Preparation of 140b: Compound 139b (0.760 g, 0.85 mmol) was stirred at ambient temperature with 4M hydrochloric acid in dioxane (30 mL). After 16 h, the completion of the reaction was confirmed by MS and the reaction mixture was concentrated and to the residue, ethyl acetate was added and the precipitated product was filtered, washed with hexanes and dried in the vacuum oven at 45° C. overnight. The pure hydrochloride salt was isolated (0.300 g, 56%) as a white powder. MS Cal. for C₃₃H₅₈N₄O₃ 2HCl; 631.76 Found: 559.4 (M+H, free base).

Preparation of 140c: Compound 139c (0.9 g, 0.9 mmol) was stirred at ambient temperature with 4M hydrochloric acid in dioxane (20 mL). After 16 h, the completion of the reaction was confirmed by MS and the reaction mixture was concentrated and to the residue, ethyl acetate was added and the precipitated product was filtered, washed with hexanes and dried in the vacuum oven at 45° C. overnight. The pure hydrochloride salt was isolated (0.508 g, 73%) as a white powder. MS Cal. for C₃₉H₇₁N₅O₃ 3HCl; 767.4 Found: 658.4 (M+H, free base).

Preparation of 142a: Compound 141a (0.826 g, 1 mmol) and HBTU (0.379 g, 1 eq) were taken in a mixture of DCM/DMF (2:1). To that DIEA (0.381 g, 3 eq.) was added, the mixture stirred for 5 minutes. Histamine (0.111 g, 1 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue is purified by chromatography to get 142a. MS Cal. for C₅₀H₇₈N₈O₈ 919.2 Found: 920.2 (M+H).

Preparation of 142b: Compound 141a (0.826 g, 1 mmol) and HBTU (0.379 g, 1 eq) were taken in a mixture of DCM/DMF (2:1). To that DIEA (0.381 g, 3 eq.) was added, the mixture stirred for 5 minutes. N,N-Dimethyl ethylene diamine (0.088 g, 1 eq) was added to that and stirred for 2 hrs at ambient temperature. The reaction mixture was added to ice-water mixture and extracted with ethyl acetate. Organic layer was dried over anhydrous sodium sulfate and removed the solvent under reduced pressure. The residue is purified by chromatography to get 142b. MS Cal. for C₄₉H₈₁N₇O₈ 896.21 Found: 897.2 (M+H).

Preparation of 142c: Compound 141a (0.826 g, 1 mmol) and HBTU (0.379 g, 1 eq.) were taken together in a mixture of DCM/DMF (2:1). To that DIEA (0.38 g, 3 eq.) was added, stirred the mixture for 5 minutes. N,N,N′,N′-Tetramethyliminobispropylamine (0.187 g, 1 mmol) was added and the mixture stirred overnight at ambient temperature. The reaction mixture was added to ice-water mixture and extracted with ethyl acetate. Organic layer was dried over anhydrous sodium sulfate and removed the solvent under reduced pressure. The residue is purified by chromatography to get 142c. MS Cal. for C₅₅H₉₄N₈O₈ 995.38 Found: 996.4 (M+H).

### Example 7

### Methods of Preparation of Nucleic Acid Association Complex with Novel Cationic Lipids for Delivery

The association complex for delivery of nucleic acids in vitro and in vivo are prepared with or with out known helper and/or fusogenic lipids, particle stabilizing lipids for example PEG-lipids and lipid like compounds as previously described (WO2006052767; US20060008910; US20060240093; WO2006074546; J. Control Release, 2006, 112, 280-290; Biochim. Biophys. Acta, 2005, 1669, 155; US20050234232; US20050222064; US20060240554; US005820873; WO98018480; US20050170508; WO2005000360; WO2005070466; WO96034876; WO98018480; US20050170508).

### Method 1: Association Complex Via Ion Pairing for Delivery of Nucleic Acids In Vitro and in Vivo.

1.1. siRNA—cationic lipid association complex: Each cationic lipid from Examples 1 to 6 is individually mixed with siRNA of interest at different N to P ratio (nitrogens on the cationic lipid to phosphate or phoshporothioate or mixed phosphate and phosphorothioate) or molar ratio to obtain ion-paired complex of siRNA and cationic lipid in PBS buffer at physiological pH for in vitro and in vivo administration of siRNA. Methods of in vivo administration are systemic, local and pulmonary via nasal administration. A solution of the lipid in ethanol is mixed with siRNA in PBS buffer to obtain the ion pair.
1.2. microRNA—cationic lipid association complex: The microRNA is mixed with each cationic lipid from the Examples 1-6 as described in Method 1.1 to obtain the ion pair complex for in vitro and in vivo delivery.
1.3. antisense oligonucleotides—cationic lipid association complex: The antisense oligonucleotide is mixed with each cationic lipid from the Examples 1-6 as described in Method 1.1 to obtain the ion pair complex for in vitro and in vivo delivery.
1.4. Aptamer—cationic lipid association complex: An aptamer is mixed with each cationic lipid from the Example 1-6 as described in Method 1.1 to obtain the ion pair complex for in vitro and in vivo delivery.
1.5. Decoy nucleic acid—cationic lipid association complex: The decoy nucleic acid is mixed with each cationic lipid from the Examples 1-6 as described in Method 1.1 to obtain the ion pair complex for in vitro and in vivo delivery.
Method 2: Association Complex with Cationic Lipid and Helper and/or Fusogenic Lipid for Delivery of Nucleic Acids In Vitro and In Vivo.
2.1. siRNA delivery: A solution of each cationic lipid from Examples 1 to 6 and a solution of 1,2-disteraoyl-sn-glycero-3-phosphocholine (DSPC) are mixed together in different ratio with siRNA to obtain a novel siRNA lipid association complex for delivery of siRNA. Titration of the cationic lipid, DSPC and siRNA at physiological pH are performed to obtain the optimum ratio between each cationic lipid from Example 1-6, DSPC and siRNA for delivery. Methods of in vivo administration are systemic, local and pulmonary via nasal administration.
2.2. microRNA: The microRNA is formulated with each cationic lipid from the Examples 1-6 and DSPC as described in Method 2.1 to obtain the corresponding formulation for in vitro and in vivo delivery.
2.3. antisense oligonucleotides: The antisense oligonucleotide is formulated with each cationic lipid from the Examples 1-6 and DSPC as described in Method 2.1 to obtain the corresponding formulation for in vitro and in vivo delivery.
2.4. Aptamer: Aptamer is formulated with each cationic lipid from the Examples 1-6 and DSPC as described in Method 2.1 to obtain the corresponding formulation for in vitro and in vivo delivery.
2.5. Decoy nucleic acid: A decoy RNA is formulated with each cationic lipid from the Examples 1-6 and DSPC as described in Method 2.1 to obtain the corresponding formulation for in vitro and in vivo delivery.
Method 3: Association Complex with Cationic Lipid and Helper and/or Fusogenic Lipid for Delivery of Nucleic Acids In Vitro and In Vivo.
3.1. siRNA delivery: A solution of each cationic lipid from Examples 1 to 6, a solution of 1,2-disteraoyl-sn-glycero-3-phosphocholine (DSPC) and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DSPC, PEG-DMG and siRNA at physiological pH are performed to obtain the optimum ratio between each cationic lipid from Example 1-6, DSPC, PEG-DMG and siRNA for delivery. Methods of in vivo administration are systemic, local and pulmonary via nasal administration.
3.2. microRNA: The microRNA is formulated with each cationic lipid from the Examples 1-6, DSPC and PEG-DMG as described in Method 3.1 to obtain the corresponding formulation for in vitro and in vivo delivery.
3.3. antisense oligonucleotides: The antisense oligonucleotides is formulated with each cationic lipid from the Examples 1-6, DSPC and PEG-DMG as described in Method 3.1 to obtain the corresponding formulation for in vitro and in vivo delivery.
3.4. Aptamer: Aptamer is formulated with each cationic lipid from the Examples 1-6, DSPC and PEG-DMG as described in Method 3.1 to obtain the corresponding formulation for in vitro and in vivo delivery.
3.5. Decoy nucleic acid: A decoy RNA formulated with each cationic lipid from the Examples 1-6, DSPC and PEG-DMG as described in Method 3.1 to obtain the corresponding formulation for in vitro and in vivo delivery.
Method 4: Association Complex with Cationic Lipid and Helper and/or Fusogenic Lipid for Delivery of Nucleic Acids In Vitro and In Vivo.
4.1. siRNA delivery: A solution of each cationic lipid from Examples 1 to 6 and a solution of cholesterol are mixed together in different ratio with siRNA to obtain a novel siRNA lipid association complex for delivery of siRNA. Titration of the cationic lipid, cholesterol and siRNA at physiological pH are performed to obtain the optimum ratio between each cationic lipid from Example 1-6, cholesterol and siRNA for delivery. Methods of in vivo administration are systemic, local and pulmonary via nasal administration.
4.2. microRNA: The microRNA is formulated with each cationic lipid from the Examples 1-6 and cholesterol as described in Method 4.1 to obtain the corresponding formulation for in vitro and in vivo delivery.
4.3. antisense oligonucleotides: The antisense oligonucleotide is formulated with each cationic lipid from the Examples 1-6 and cholesterol as described in Method 4.1 to obtain the corresponding formulation for in vitro and in vivo delivery.
4.4. Aptamer: Aptamer is formulated with each cationic lipid from the Examples 1-6 and cholesterol as described in Method 4.1 to obtain the corresponding formulation for in vitro and in vivo delivery.
4.5. Decoy nucleic acid: A decoy RNA is formulated with each cationic lipid from the Examples 1-6 and cholesterol as described in Method 4.1 to obtain the corresponding formulation for in vitro and in vivo delivery.
Method 5: Association Complex with Cationic Lipid and Helper and/or Fusogenic Lipid for Delivery of Nucleic Acids In Vitro and In Vivo.
5.1. siRNA delivery: A solution of each cationic lipid from Examples 1 to 6, a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, cholesterol, PEG-DMG and siRNA at physiological pH are performed to obtain the optimum ratio between each cationic lipid from Example 1-6, cholesterol, PEG-DMG and siRNA for delivery. Methods of in vivo administration are systemic, local and pulmonary via nasal administration.
5.2. microRNA: The microRNA is formulated with each cationic lipid from the Examples 1-6, cholesterol and PEG-DMG as described in Method 5.1 to obtain the corresponding formulation for in vitro and in vivo delivery.
5.3. antisense oligonucleotides: The antisense oligonucleotides is formulated with each cationic lipid from the Examples 1-6, cholesterol and PEG-DMG as described in Method 5.1 to obtain the corresponding formulation for in vitro and in vivo delivery.
5.4. Aptamer: Aptamer is formulated with each cationic lipid from the Examples 1-6, cholesterol and PEG-DMG as described in Method 5.1 to obtain the corresponding formulation for in vitro and in vivo delivery.
5.5. Decoy nucleic acid: A decoy RNA formulated with each cationic lipid from the Examples 1-6, cholesterol and PEG-DMG as described in Method 5.1 to obtain the corresponding formulation for in vitro and in vivo delivery.
Method 6: Association Complex with Cationic Lipid and Helper and/or Fusogenic Lipid for Delivery of Nucleic Acids In Vitro and In Vivo.
6.1. siRNA delivery: A solution of each cationic lipid from Examples 1 to 6 and a solution of 1,2-palmitoyl-sn-glycero-3-phosphoethanolamine (DPPE) are mixed together in different ratio with siRNA to obtain a novel siRNA lipid association complex for delivery of siRNA. Titration of the cationic lipid, DPPE and siRNA at physiological pH are performed to obtain the optimum ratio between each cationic lipid from Example 1-6, DPPE and siRNA for delivery. Methods of in vivo administration are systemic, local and pulmonary via nasal administration.
6.2. microRNA: The microRNA is formulated with each cationic lipid from the Examples 1-6 and DPPE as described in Method 6.1 to obtain the corresponding formulation for in vitro and in vivo delivery.
6.3. antisense oligonucleotides: The antisense oligonucleotide is formulated with each cationic lipid from the Examples 1-6 and DPPE as described in Method 6.1 to obtain the corresponding formulation for in vitro and in vivo delivery.
6.4. Aptamer: Aptamer is formulated with each cationic lipid from the Examples 1-6 and DPPE as described in Method 6.1 to obtain the corresponding formulation for in vitro and in vivo delivery.
6.5. Decoy nucleic acid: A decoy RNA is formulated with each cationic lipid from the Examples 1-6 and DPPE as described in Method 6.1 to obtain the corresponding formulation for in vitro and in vivo delivery.
Method 7: Association Complex with Cationic Lipid and Helper and/or Fusogenic Lipid for Delivery of Nucleic Acids In Vitro and In Vivo.
7.1. siRNA delivery: A solution of each cationic lipid from Examples 1 to 6, a solution of 1,2-palmitoyl-sn-glycero-3-phosphoethanolamine (DPPE) and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DPPE, PEG-DMG and siRNA at physiological pH are performed to obtain the optimum ratio between each cationic lipid from Example 1-6, DPPE, PEG-DMG and siRNA for delivery. Methods of in vivo administration are systemic, local and pulmonary via nasal administration.
7.2. microRNA: The microRNA is formulated with each cationic lipid from the Examples 1-6, DPPE and PEG-DMG as described in Method 7.1 to obtain the corresponding formulation for in vitro and in vivo delivery.
7.3. antisense oligonucleotides: The antisense oligonucleotides is formulated with each cationic lipid from the Examples 1-6, DPPE and PEG-DMG as described in Method 7.1 to obtain the corresponding formulation for in vitro and in vivo delivery.
7.4. Aptamer: Aptamer is formulated with each cationic lipid from the Examples 1-6, DPPE and PEG-DMG as described in Method 7.1 to obtain the corresponding formulation for in vitro and in vivo delivery.
7.5. Decoy nucleic acid: A decoy RNA formulated with each cationic lipid from the Examples 1-6, DPPE and PEG-DMG as described in Method 7.1 to obtain the corresponding formulation for in vitro and in vivo delivery.
Method 8: Association Complex with Cationic Lipid and Helper and/or Fusogenic Lipid for Delivery of Nucleic Acids In Vitro and In Vivo.
8.1. siRNA delivery: A solution of each cationic lipid from Examples 1 to 6 and a solution of 1,2-oleoyl-sn-glycero-3-phosphoethanolamine (DOPE) are mixed together in different ratio with siRNA to obtain a novel siRNA lipid association complex for delivery of siRNA. Titration of the cationic lipid, DOPE and siRNA at physiological pH are performed to obtain the optimum ratio between each cationic lipid from Example 1-6, DOPE and siRNA for delivery. Methods of in vivo administration are systemic, local and pulmonary via nasal administration.
8.2. microRNA: The microRNA is formulated with each cationic lipid from the Examples 1-6 and DOPE as described in Method 8.1 to obtain the corresponding formulation for in vitro and in vivo delivery.
8.3. antisense oligonucleotides: The antisense oligonucleotide is formulated with each cationic lipid from the Examples 1-6 and DOPE as described in Method 8.1 to obtain the corresponding formulation for in vitro and in vivo delivery.
8.4. Aptamer: Aptamer is formulated with each cationic lipid from the Examples 1-6 and DOPE as described in Method 8.1 to obtain the corresponding formulation for in vitro and in vivo delivery.
8.5. Decoy nucleic acid: A decoy RNA is formulated with each cationic lipid from the Examples 1-6 and DOPE as described in Method 8.1 to obtain the corresponding formulation for in vitro and in vivo delivery.
Method 9: Association Complex with Cationic Lipid and Helper and/or Fusogenic Lipid for Delivery of Nucleic Acids In Vitro and In Vivo.
9.1. siRNA delivery: A solution of each cationic lipid from Examples 1 to 6, a solution of 1,2-oleoyl-sn-glycero-3-phosphoethanolamine (DOPE) and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DOPE, PEG-DMG and siRNA at physiological pH are performed to obtain the optimum ratio between each cationic lipid from Example 1-6, DOPE, PEG-DMG and siRNA for delivery. Methods of in vivo administration are systemic, local and pulmonary via nasal administration.
9.2. microRNA: The microRNA is formulated with each cationic lipid from the Examples 1-6, DOPE and PEG-DMG as described in Method 9.1 to obtain the corresponding formulation for in vitro and in vivo delivery.
9.3. antisense oligonucleotides: The antisense oligonucleotides is formulated with each cationic lipid from the Examples 1-6, DOPE and PEG-DMG as described in Method 5.1 to obtain the corresponding formulation for in vitro and in vivo delivery.
9.4. Aptamer: Aptamer is formulated with each cationic lipid from the Examples 1-6, DOPE and PEG-DMG as described in Method 9.1 to obtain the corresponding formulation for in vitro and in vivo delivery.
9.5. Decoy nucleic acid: A decoy RNA formulated with each cationic lipid from the Examples 1-6, DOPE and PEG-DMG as described in Method 9.1 to obtain the corresponding formulation for in vitro and in vivo delivery
Method 10: Association Complex with Cationic Lipid and Helper and/or Fusogenic Lipid for Delivery of Nucleic Acids In Vitro and In Vivo.
10.1. siRNA delivery: A solution of each cationic lipid from Examples 1 to 6, a solution of 1,2-disteraoyl-sn-glycero-3-phosphocholine (DSPC) and cholesterol are mixed together in different ratio with siRNA to obtain a novel siRNA lipid association complex for delivery of siRNA. Titration of the cationic lipid, DSPC and siRNA at physiological pH are performed to obtain the optimum ratio between each cationic lipid from Example 1-6, DSPC, cholesterol and siRNA for delivery. Methods of in vivo administration are systemic, local and pulmonary via nasal administration.
10.2. microRNA: The microRNA is formulated with each cationic lipid from the Examples 1-6, DSPC and cholesterol as described in Method 10.1 to obtain the corresponding formulation for in vitro and in vivo delivery.
10.3. antisense oligonucleotides: The antisense oligonucleotide is formulated with each cationic lipid from the Examples 1-6, DSPC and cholesterol as described in Method 10.1 to obtain the corresponding formulation for in vitro and in vivo delivery.
10.4. Aptamer: Aptamer is formulated with each cationic lipid from the Examples 1-6, DSPC and cholesterol as described in Method 10.1 to obtain the corresponding formulation for in vitro and in vivo delivery.
10.5. Decoy nucleic acid: A decoy RNA is formulated with each cationic lipid from the Examples 1-6, DSPC and cholesterol as described in Method 10.1 to obtain the corresponding formulation for in vitro and in vivo delivery.
Method 11: Association Complex with Cationic Lipid and Helper and/or Fusogenic Lipid for Delivery of Nucleic Acids In Vitro and In Vivo.
11.1. siRNA delivery: A solution of each cationic lipid from Examples 1 to 6, a solution of 1,2-disteraoyl-sn-glycero-3-phosphocholine (DSPC), a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DSPC, cholesterol, PEG-DMG and siRNA at physiological pH are performed to obtain the optimum ratio between each cationic lipid from Example 1-6, DSPC, cholesterol, PEG-DMG and siRNA for delivery. Methods of in vivo administration are systemic, local and pulmonary via nasal administration.
11.2. microRNA: The microRNA is formulated with each cationic lipid from the Examples 1-6, DSPC, cholesterol and PEG-DMG as described in Method 11.1 to obtain the corresponding formulation for in vitro and in vivo delivery.
11.3. antisense oligonucleotides: The antisense oligonucleotides is formulated with each cationic lipid from the Examples 1-6, DSPC, cholesterol and PEG-DMG as described in Method 11.1 to obtain the corresponding formulation for in vitro and in vivo delivery.
11.4. Aptamer: Aptamer is formulated with each cationic lipid from the Examples 1-6, DSPC, cholesterol and PEG-DMG as described in Method 11.1 to obtain the corresponding formulation for in vitro and in vivo delivery.
11.5. Decoy nucleic acid: A decoy RNA formulated with each cationic lipid from the Examples 1-6, DSPC, cholesterol and PEG-DMG as described in Method 11.1 to obtain the corresponding formulation for in vitro and in vivo delivery.
Method 12: Association Complex with Cationic Lipid and Helper and/or Fusogenic Lipid for Delivery of Nucleic Acids.
12.1. siRNA delivery: A solution of each cationic lipid from Examples 1 to 6, a solution of 1,2-palmitoyl-sn-glycero-3-phosphoethanolamine (DPPE) and a solution of cholesterol are mixed together in different ratio with siRNA to obtain a novel siRNA lipid association complex for delivery of siRNA. Titration of the cationic lipid, DPPE, cholesterol and siRNA at physiological pH are performed to obtain the optimum ratio between each cationic lipid from Example 1-6, DPPE, cholesterol and siRNA for delivery. Methods of in vivo administration are systemic, local and pulmonary via nasal administration.
12.2. microRNA: The microRNA is formulated with each cationic lipid from the Examples 1-6, DPPE and cholesterol as described in Method 12.1 to obtain the corresponding formulation for in vitro and in vivo delivery.
12.3. antisense oligonucleotides: The antisense oligonucleotide is formulated with each cationic lipid from the Examples 1-6, DPPE and cholesterol as described in Method 12.1 to obtain the corresponding formulation for in vitro and in vivo delivery.
12.4. Aptamer: Aptamer is formulated with each cationic lipid from the Examples 1-6, DPPE and cholesterol as described in Method 12.1 to obtain the corresponding formulation for in vitro and in vivo delivery.
12.5. Decoy nucleic acid: A decoy RNA is formulated with each cationic lipid from the Examples 1-6, DPPE and cholesterol as described in Method 12.1 to obtain the corresponding formulation for in vitro and in vivo delivery.
Method 13: Association Complex with Cationic Lipid and Helper and/or Fusogenic Lipid for Delivery of Nucleic Acids.
13.1. siRNA delivery: A solution of each cationic lipid from Examples 1 to 6, a solution of 1,2-palmitoyl-sn-glycero-3-phosphoethanolamine (DPPE), a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DPPE, cholesterol, PEG-DMG and siRNA at physiological pH are performed to obtain the optimum ratio between each cationic lipid from Example 1-6, DPPE, cholesterol, PEG-DMG and siRNA for delivery. Methods of in vivo administration are systemic, local and pulmonary via nasal administration.
13.2. microRNA: The microRNA is formulated with each cationic lipid from the Examples 1-6, DPPE, cholesterol and PEG-DMG as described in Method 13.1 to obtain the corresponding formulation for in vitro and in vivo delivery.
13.3. antisense oligonucleotides: The antisense oligonucleotides is formulated with each cationic lipid from the Examples 1-6, DPPE, cholesterol and PEG-DMG as described in Method 13.1 to obtain the corresponding formulation for in vitro and in vivo delivery.
13.4. Aptamer: Aptamer is formulated with each cationic lipid from the Examples 1-6, DPPE, cholesterol and PEG-DMG as described in Method 13.1 to obtain the corresponding formulation for in vitro and in vivo delivery.
13.5. Decoy nucleic acid: A decoy RNA formulated with each cationic lipid from the Examples 1-6, DPPE, cholesterol and PEG-DMG as described in Method 13.1 to obtain the corresponding formulation for in vitro and in vivo delivery.
Method 14: Association Complex with Cationic Lipid and Helper and/or Fusogenic Lipid for Delivery of Nucleic Acids In Vitro and In Vivo.
14.1. siRNA delivery: A solution of each cationic lipid from Examples 1 to 6, a solution of 1,2-oleoyl-sn-glycero-3-phosphoethanolamine (DOPE) and a solution of cholesterol are mixed together in different ratio with siRNA to obtain a novel siRNA lipid association complex for delivery of siRNA. Titration of the cationic lipid, DOPE, cholesterol and siRNA at physiological pH are performed to obtain the optimum ratio between each cationic lipid from Example 1-6, DOPE, cholesterol and siRNA for delivery. Methods of in vivo administration are systemic, local and pulmonary via nasal administration.
14.2. microRNA: The microRNA is formulated with each cationic lipid from the Examples 1-6, DOPE and cholesterol as described in Method 14.1 to obtain the corresponding formulation for in vitro and in vivo delivery.
14.3. antisense oligonucleotides: The antisense oligonucleotide is formulated with each cationic lipid from the Examples 1-6, DOPE and cholesterol as described in Method 14.1 to obtain the corresponding formulation for in vitro and in vivo delivery.
14.4. Aptamer: Aptamer is formulated with each cationic lipid from the Examples 1-6, DOPE and cholesterol as described in Method 14.1 to obtain the corresponding formulation for in vitro and in vivo delivery.
14.5. Decoy nucleic acid: A decoy RNA is formulated with each cationic lipid from the Examples 1-6, DOPE and cholesterol as described in Method 14.1 to obtain the corresponding formulation for in vitro and in vivo delivery.
Method 15: Association Complex with Cationic Lipid and Helper and/or Fusogenic Lipid for Delivery of Nucleic Acids.
15.1. siRNA delivery: A solution of each cationic lipid from Examples 1 to 6, a solution of 1,2-oleoyl-sn-glycero-3-phosphoethanolamine (DOPE), a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DOPE, cholesterol, PEG-DMG and siRNA at physiological pH are performed to obtain the optimum ratio between each cationic lipid from Example 1-6, DOPE, cholesterol, PEG-DMG and siRNA for delivery. Methods of in vivo administration are systemic, local and pulmonary via nasal administration.
15.2. microRNA: The microRNA is formulated with each cationic lipid from the Examples 1-6, DOPE, cholesterol and PEG-DMG as described in Method 15.1 to obtain the corresponding formulation for in vitro and in vivo delivery.
15.3. antisense oligonucleotides: The antisense oligonucleotides is formulated with each cationic lipid from the Examples 1-6, DOPE, cholesterol and PEG-DMG as described in Method 15.1 to obtain the corresponding formulation for in vitro and in vivo delivery.
15.4. Aptamer: Aptamer is formulated with each cationic lipid from the Examples 1-6, DOPE, cholesterol and PEG-DMG as described in Method 15.1 to obtain the corresponding formulation for in vitro and in vivo delivery.
15.5. Decoy nucleic acid: A decoy RNA formulated with each cationic lipid from the Examples 1-6, DOPE, cholesterol and PEG-DMG as described in Method 15.1 to obtain the corresponding formulation for in vitro and in vivo delivery.
Method 16: Association Complex with Cationic Lipid and Helper and/or Fusogenic Lipid for Delivery of Nucleic Acids In Vitro and In Vivo.
16.1. siRNA delivery: A solution of each cationic lipid from Examples 1 to 6 and a solution of 1,2-disteraoyl-sn-glycero-3-phosphocholine (DSPC) are mixed together in different ratio with siRNA to obtain a novel siRNA lipid association complex for delivery of siRNA. Titration of the cationic lipid, DSPC and siRNA at physiological pH are performed to obtain the optimum ratio between each cationic lipid from Example 1-6, DSPC and siRNA for delivery. Methods of in vivo administration are systemic, local and pulmonary via nasal administration.
16.2. microRNA: The microRNA is formulated with each cationic lipid from the Examples 1-6 and DSPC as described in Method 16.1 to obtain the corresponding formulation for in vitro and in vivo delivery.
16.3. antisense oligonucleotides: The antisense oligonucleotide is formulated with each cationic lipid from the Examples 1-6 and DSPC as described in Method 16.1 to obtain the corresponding formulation for in vitro and in vivo delivery.
16.4. Aptamer: Aptamer is formulated with each cationic lipid from the Examples 1-6 and DSPC as described in Method 2.1 to obtain the corresponding formulation for in vitro and in vivo delivery.
16.5. Decoy nucleic acid: A decoy RNA is formulated with each cationic lipid from the Examples 1-6 and DSPC as described in Method 16.1 to obtain the corresponding formulation for in vitro and in vivo delivery.
Method 17: Association Complex with Cationic Lipid and Helper and/or Fusogenic Lipid for Delivery of Nucleic Acids In Vitro and In Vivo.
17.1. siRNA delivery: A solution of each cationic lipid from Examples 1 to 6, a solution of 1,2-disteraoyl-sn-glycero-3-phosphocholine (DSPC) and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DSPC, PEG-DMG and siRNA at physiological pH are performed to obtain the optimum ratio between each cationic lipid from Example 1-6, DSPC, PEG-DMG and siRNA for delivery. Methods of in vivo administration are systemic, local and pulmonary via nasal administration.
17.2. microRNA: The microRNA is formulated with each cationic lipid from the Examples 1-6, DSPC and PEG-DMG as described in Method 17.1 to obtain the corresponding formulation for in vitro and in vivo delivery.
17.3. antisense oligonucleotides: The antisense oligonucleotides is formulated with each cationic lipid from the Examples 1-6, DSPC and PEG-DMG as described in Method 3.1 to obtain the corresponding formulation for in vitro and in vivo delivery.
17.4. Aptamer: Aptamer is formulated with each cationic lipid from the Examples 1-6, DSPC and PEG-DMG as described in Method 17.1 to obtain the corresponding formulation for in vitro and in vivo delivery.
17.5. Decoy nucleic acid: A decoy RNA formulated with each cationic lipid from the Examples 1-6, DSPC and PEG-DMG as described in Method 17.1 to obtain the corresponding formulation for in vitro and in vivo delivery.
Method 18: Association Complex with Cationic Lipid and Helper and/or Fusogenic Lipid for Delivery of Nucleic Acids In Vitro and In Vivo.
18.1. siRNA delivery: A solution of each cationic lipid from Examples 1 to 6 and a solution of cholesterol are mixed together in different ratio with siRNA to obtain a novel siRNA lipid association complex for delivery of siRNA. Titration of the cationic lipid, cholesterol and siRNA at physiological pH are performed to obtain the optimum ratio between each cationic lipid from Example 1-6, cholesterol and siRNA for delivery. Methods of in vivo administration are systemic, local and pulmonary via nasal administration.
18.2. microRNA: The microRNA is formulated with each cationic lipid from the Examples 1-6 and cholesterol as described in Method 18.1 to obtain the corresponding formulation for in vitro and in vivo delivery.
18.3. antisense oligonucleotides: The antisense oligonucleotide is formulated with each cationic lipid from the Examples 1-6 and cholesterol as described in Method 18.1 to obtain the corresponding formulation for in vitro and in vivo delivery.
18.4. Aptamer: Aptamer is formulated with each cationic lipid from the Examples 1-6 and cholesterol as described in Method 18.1 to obtain the corresponding formulation for in vitro and in vivo delivery.
18.5. Decoy nucleic acid: A decoy RNA is formulated with each cationic lipid from the Examples 1-6 and cholesterol as described in Method 18.1 to obtain the corresponding formulation for in vitro and in vivo delivery.
Method 19: Association Complex with Cationic Lipid and Helper and/or Fusogenic Lipid for Delivery of Nucleic Acids In Vitro and In Vivo.
19.1. siRNA delivery: A solution of each cationic lipid from Examples 1 to 6, a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, cholesterol, PEG-DMG and siRNA at physiological pH are performed to obtain the optimum ratio between each cationic lipid from Example 1-6, cholesterol, PEG-DMG and siRNA for delivery. Methods of in vivo administration are systemic, local and pulmonary via nasal administration.
19.2. microRNA: The microRNA is formulated with each cationic lipid from the Examples 1-6, cholesterol and PEG-DMG as described in Method 19.1 to obtain the corresponding formulation for in vitro and in vivo delivery.
19.3. antisense oligonucleotides: The antisense oligonucleotides is formulated with each cationic lipid from the Examples 1-6, cholesterol and PEG-DMG as described in Method 5.1 to obtain the corresponding formulation for in vitro and in vivo delivery.
19.4. Aptamer: Aptamer is formulated with each cationic lipid from the Examples 1-6, cholesterol and PEG-DMG as described in Method 19.1 to obtain the corresponding formulation for in vitro and in vivo delivery.
19.5. Decoy nucleic acid: A decoy RNA formulated with each cationic lipid from the Examples 1-6, cholesterol and PEG-DMG as described in Method 19.1 to obtain the corresponding formulation for in vitro and in vivo delivery.
Method 20: Association Complex with Cationic Lipid and Helper and/or Fusogenic Lipid for Delivery of Nucleic Acids In Vitro and In Vivo.
20.1. siRNA delivery: A solution of each cationic lipid from Examples 1 to 6 and a solution of 1,2-palmitoyl-sn-glycero-3-phosphoethanolamine (DPPE) are mixed together in different ratio with siRNA to obtain a novel siRNA lipid association complex for delivery of siRNA. Titration of the cationic lipid, DPPE and siRNA at physiological pH are performed to obtain the optimum ratio between each cationic lipid from Example 1-6, DPPE and siRNA for delivery. Methods of in vivo administration are systemic, local and pulmonary via nasal administration.
20.2. microRNA: The microRNA is formulated with each cationic lipid from the Examples 1-6 and DPPE as described in Method 20.1 to obtain the corresponding formulation for in vitro and in vivo delivery.
20.3. antisense oligonucleotides: The antisense oligonucleotide is formulated with each cationic lipid from the Examples 1-6 and DPPE as described in Method 20.1 to obtain the corresponding formulation for in vitro and in vivo delivery.
20.4. Aptamer: Aptamer is formulated with each cationic lipid from the Examples 1-6 and DPPE as described in Method 20.1 to obtain the corresponding formulation for in vitro and in vivo delivery.
20.5. Decoy nucleic acid: A decoy RNA is formulated with each cationic lipid from the Examples 1-6 and DPPE as described in Method 20.1 to obtain the corresponding formulation for in vitro and in vivo delivery.
Method 21: Association Complex with Cationic Lipid and Helper and/or Fusogenic Lipid for Delivery of Nucleic Acids In Vitro and In Vivo.
21.1. siRNA delivery: A solution of each cationic lipid from Examples 1 to 6, a solution of 1,2-palmitoyl-sn-glycero-3-phosphoethanolamine (DPPE) and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DPPE, PEG-DMG and siRNA at physiological pH are performed to obtain the optimum ratio between each cationic lipid from Example 1-6, DPPE, PEG-DMG and siRNA for delivery. Methods of in vivo administration are systemic, local and pulmonary via nasal administration.
21.2. microRNA: The microRNA is formulated with each cationic lipid from the Examples 1-6, DPPE and PEG-DMG as described in Method 21.1 to obtain the corresponding formulation for in vitro and in vivo delivery.
21.3. antisense oligonucleotides: The antisense oligonucleotides is formulated with each cationic lipid from the Examples 1-6, DPPE and PEG-DMG as described in Method 21.1 to obtain the corresponding formulation for in vitro and in vivo delivery.
21.4. Aptamer: Aptamer is formulated with each cationic lipid from the Examples 1-6, DPPE and PEG-DMG as described in Method 21.1 to obtain the corresponding formulation for in vitro and in vivo delivery.
21.5. Decoy nucleic acid: A decoy RNA formulated with each cationic lipid from the Examples 1-6, DPPE and PEG-DMG as described in Method 21.1 to obtain the corresponding formulation for in vitro and in vivo delivery.
Method 22: Association Complex with Cationic Lipid and Helper and/or Fusogenic Lipid for Delivery of Nucleic Acids In Vitro and In Vivo.
22.1. siRNA delivery: A solution of each cationic lipid from Examples 1 to 6 and a solution of 1,2-oleoyl-sn-glycero-3-phosphoethanolamine (DOPE) are mixed together in different ratio with siRNA to obtain a novel siRNA lipid association complex for delivery of siRNA. Titration of the cationic lipid, DOPE and siRNA at physiological pH are performed to obtain the optimum ratio between each cationic lipid from Example 1-6, DOPE and siRNA for delivery. Methods of in vivo administration are systemic, local and pulmonary via nasal administration.
22.2. microRNA: The microRNA is formulated with each cationic lipid from the Examples 1-6 and DOPE as described in Method 22.1 to obtain the corresponding formulation for in vitro and in vivo delivery.
22.3. antisense oligonucleotides: The antisense oligonucleotide is formulated with each cationic lipid from the Examples 1-6 and DOPE as described in Method 22.1 to obtain the corresponding formulation for in vitro and in vivo delivery.
22.4. Aptamer: Aptamer is formulated with each cationic lipid from the Examples 1-6 and DOPE as described in Method 22.1 to obtain the corresponding formulation for in vitro and in vivo delivery.
22.5. Decoy nucleic acid: A decoy RNA is formulated with each cationic lipid from the Examples 1-6 and DOPE as described in Method 22.1 to obtain the corresponding formulation for in vitro and in vivo delivery.
Method 23: Association Complex with Cationic Lipid and Helper and/or Fusogenic Lipid for Delivery of Nucleic Acids In Vitro and In Vivo.
23.1. siRNA delivery: A solution of each cationic lipid from Examples 1 to 6, a solution of 1,2-oleoyl-sn-glycero-3-phosphoethanolamine (DOPE) and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DOPE, PEG-DMG and siRNA at physiological pH are performed to obtain the optimum ratio between each cationic lipid from Example 1-6, DOPE, PEG-DMG and siRNA for delivery. Methods of in vivo administration are systemic, local and pulmonary via nasal administration.
23.2. microRNA: The microRNA is formulated with each cationic lipid from the Examples 1-6, DOPE and PEG-DMG as described in Method 23.1 to obtain the corresponding formulation for in vitro and in vivo delivery.
23.3. antisense oligonucleotides: The antisense oligonucleotides is formulated with each cationic lipid from the Examples 1-6, DOPE and PEG-DMG as described in Method 5.1 to obtain the corresponding formulation for in vitro and in vivo delivery.
23.4. Aptamer: Aptamer is formulated with each cationic lipid from the Examples 1-6, DOPE and PEG-DMG as described in Method 23.1 to obtain the corresponding formulation for in vitro and in vivo delivery.
23.5. Decoy nucleic acid: A decoy RNA formulated with each cationic lipid from the Examples 1-6, DOPE and PEG-DMG as described in Method 23.1 to obtain the corresponding formulation for in vitro and in vivo delivery
Method 24: Association Complex with Cationic Lipid and Helper and/or Fusogenic Lipid for Delivery of Nucleic Acids In Vitro and In Vivo.
24.1. siRNA delivery: A solution of each cationic lipid from Examples 1 to 6, a solution of 1,2-disteraoyl-sn-glycero-3-phosphocholine (DSPC) and cholesterol are mixed together in different ratio with siRNA to obtain a novel siRNA lipid association complex for delivery of siRNA. Titration of the cationic lipid, DSPC and siRNA at physiological pH are performed to obtain the optimum ratio between each cationic lipid from Example 1-6, DSPC, cholesterol and siRNA for delivery. Methods of in vivo administration are systemic, local and pulmonary via nasal administration.
24.2. microRNA: The microRNA is formulated with each cationic lipid from the Examples 1-6, DSPC and cholesterol as described in Method 24.1 to obtain the corresponding formulation for in vitro and in vivo delivery.
24.3. antisense oligonucleotides: The antisense oligonucleotide is formulated with each cationic lipid from the Examples 1-6, DSPC and cholesterol as described in Method 24.1 to obtain the corresponding formulation for in vitro and in vivo delivery.
24.4. Aptamer: Aptamer is formulated with each cationic lipid from the Examples 1-6, DSPC and cholesterol as described in Method 24.1 to obtain the corresponding formulation for in vitro and in vivo delivery.
24.5. Decoy nucleic acid: A decoy RNA is formulated with each cationic lipid from the Examples 1-6, DSPC and cholesterol as described in Method 24.1 to obtain the corresponding formulation for in vitro and in vivo delivery.
Method 25: Association Complex with Cationic Lipid and Helper and/or Fusogenic Lipid for Delivery of Nucleic Acids In Vitro and In Vivo.
25.1. siRNA delivery: A solution of each cationic lipid from Examples 1 to 6, a solution of 1,2-disteraoyl-sn-glycero-3-phosphocholine (DSPC), a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DSPC, cholesterol, PEG-DMG and siRNA at physiological pH are performed to obtain the optimum ratio between each cationic lipid from Example 1-6, DSPC, cholesterol, PEG-DMG and siRNA for delivery. Methods of in vivo administration are systemic, local and pulmonary via nasal administration.
25.2. microRNA: The microRNA is formulated with each cationic lipid from the Examples 1-6, DSPC, cholesterol and PEG-DMG as described in Method 25.1 to obtain the corresponding formulation for in vitro and in vivo delivery.
25.3. antisense oligonucleotides: The antisense oligonucleotides is formulated with each cationic lipid from the Examples 1-6, DSPC, cholesterol and PEG-DMG as described in Method 25.1 to obtain the corresponding formulation for in vitro and in vivo delivery.
25.4. Aptamer: Aptamer is formulated with each cationic lipid from the Examples 1-6, DSPC, cholesterol and PEG-DMG as described in Method 25.1 to obtain the corresponding formulation for in vitro and in vivo delivery.
25.5. Decoy nucleic acid: A decoy RNA formulated with each cationic lipid from the Examples 1-6, DSPC, cholesterol and PEG-DMG as described in Method 25.1 to obtain the corresponding formulation for in vitro and in vivo delivery.
Method 26: Association Complex with Cationic Lipid and Helper and/or Fusogenic Lipid for Delivery of Nucleic Acids.
26.1. siRNA delivery: A solution of each cationic lipid from Examples 1 to 6, a solution of 1,2-palmitoyl-sn-glycero-3-phosphoethanolamine (DPPE) and a solution of cholesterol are mixed together in different ratio with siRNA to obtain a novel siRNA lipid association complex for delivery of siRNA. Titration of the cationic lipid, DPPE, cholesterol and siRNA at physiological pH are performed to obtain the optimum ratio between each cationic lipid from Example 1-6, DPPE, cholesterol and siRNA for delivery. Methods of in vivo administration are systemic, local and pulmonary via nasal administration.
26.2. microRNA: The microRNA is formulated with each cationic lipid from the Examples 1-6, DPPE and cholesterol as described in Method 26.1 to obtain the corresponding formulation for in vitro and in vivo delivery.
26.3. antisense oligonucleotides: The antisense oligonucleotide is formulated with each cationic lipid from the Examples 1-6, DPPE and cholesterol as described in Method 26.1 to obtain the corresponding formulation for in vitro and in vivo delivery.
26.4. Aptamer: Aptamer is formulated with each cationic lipid from the Examples 1-6, DPPE and cholesterol as described in Method 26.1 to obtain the corresponding formulation for in vitro and in vivo delivery.
26.5. Decoy nucleic acid: A decoy RNA is formulated with each cationic lipid from the Examples 1-6, DPPE and cholesterol as described in Method 26.1 to obtain the corresponding formulation for in vitro and in vivo delivery.
Method 27: Association Complex with Cationic Lipid and Helper and/or Fusogenic Lipid for Delivery of Nucleic Acids.
27.1. siRNA delivery: A solution of each cationic lipid from Examples 1 to 6, a solution of 1,2-palmitoyl-sn-glycero-3-phosphoethanolamine (DPPE), a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DPPE, cholesterol, PEG-DMG and siRNA at physiological pH are performed to obtain the optimum ratio between each cationic lipid from Example 1-6, DPPE, cholesterol, PEG-DMG and siRNA for delivery. Methods of in vivo administration are systemic, local and pulmonary via nasal administration.
27.2. microRNA: The microRNA is formulated with each cationic lipid from the Examples 1-6, DPPE, cholesterol and PEG-DMG as described in Method 27.1 to obtain the corresponding formulation for in vitro and in vivo delivery.
27.3. antisense oligonucleotides: The antisense oligonucleotides is formulated with each cationic lipid from the Examples 1-6, DPPE, cholesterol and PEG-DMG as described in Method 27.1 to obtain the corresponding formulation for in vitro and in vivo delivery.
27.4. Aptamer: Aptamer is formulated with each cationic lipid from the Examples 1-6, DPPE, cholesterol and PEG-DMG as described in Method 27.1 to obtain the corresponding formulation for in vitro and in vivo delivery.
27.5. Decoy nucleic acid: A decoy RNA formulated with each cationic lipid from the Examples 1-6, DPPE, cholesterol and PEG-DMG as described in Method 27.1 to obtain the corresponding formulation for in vitro and in vivo delivery.
Method 28: Association Complex with Cationic Lipid and Helper and/or Fusogenic Lipid for Delivery of Nucleic Acids In Vitro and In Vivo.
28.1. siRNA delivery: A solution of each cationic lipid from Examples 1 to 6, a solution of 1,2-oleoyl-sn-glycero-3-phosphoethanolamine (DOPE) and a solution of cholesterol are mixed together in different ratio with siRNA to obtain a novel siRNA lipid association complex for delivery of siRNA. Titration of the cationic lipid, DOPE, cholesterol and siRNA at physiological pH are performed to obtain the optimum ratio between each cationic lipid from Example 1-6, DOPE, cholesterol and siRNA for delivery. Methods of in vivo administration are systemic, local and pulmonary via nasal administration.
28.2. microRNA: The microRNA is formulated with each cationic lipid from the Examples 1-6, DOPE and cholesterol as described in Method 28.1 to obtain the corresponding formulation for in vitro and in vivo delivery.
28.3. antisense oligonucleotides: The antisense oligonucleotide is formulated with each cationic lipid from the Examples 1-6, DOPE and cholesterol as described in Method 28.1 to obtain the corresponding formulation for in vitro and in vivo delivery.
28.4. Aptamer: Aptamer is formulated with each cationic lipid from the Examples 1-6, DOPE and cholesterol as described in Method 28.1 to obtain the corresponding formulation for in vitro and in vivo delivery.
28.5. Decoy nucleic acid: A decoy RNA is formulated with each cationic lipid from the Examples 1-6, DOPE and cholesterol as described in Method 28.1 to obtain the corresponding formulation for in vitro and in vivo delivery.
Method 29: Association Complex with Cationic Lipid and Helper and/or Fusogenic Lipid for Delivery of Nucleic Acids.
29.1. siRNA delivery: A solution of each cationic lipid from Examples 1 to 6, a solution of 1,2-oleoyl-sn-glycero-3-phosphoethanolamine (DOPE), a solution of cholesterol and a solution of PEG-Lipid (for example, (R)-Methoxy-PEG2000-carbamoyl-di-O-myristyl-sn-glyceride, PEG-DMG) are mixed together in different ratio with siRNA to obtain a novel siRNA lipid association complex for delivery of siRNA. Titration of the cationic lipid, DOPE, cholesterol, PEG-DMG and siRNA at physiological pH are performed to obtain the optimum ratio between each cationic lipid from Example 1-6, DOPE, cholesterol, PEG-DMG and siRNA for delivery. Methods of in vivo administration are systemic, local and pulmonary via nasal administration.
29.2. microRNA: The microRNA is formulated with each cationic lipid from the Examples 1-6, DOPE, cholesterol and PEG-DMG as described in Method 29.1 to obtain the corresponding formulation for in vitro and in vivo delivery.
29.3. antisense oligonucleotides: The antisense oligonucleotides is formulated with each cationic lipid from the Examples 1-6, DOPE, cholesterol and PEG-DMG as described in Method 29.1 to obtain the corresponding formulation for in vitro and in vivo delivery.
29.4. Aptamer: Aptamer is formulated with each cationic lipid from the Examples 1-6, DOPE, cholesterol and PEG-DMG as described in Method 29.1 to obtain the corresponding formulation for in vitro and in vivo delivery.
29.5. Decoy nucleic acid: A decoy RNA formulated with each cationic lipid from the Examples 1-6, DOPE, cholesterol and PEG-DMG as described in Method 29.1 to obtain the corresponding formulation for in vitro and in vivo delivery.

What is claimed is:

1. A compound of formula (I) 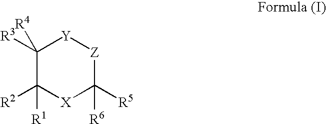
wherein:
X is NR⁷ or CH₂;
Y is NR⁸, O, S, CR⁹R¹⁰, or absent;
Z is CR¹¹R¹² or absent;
each of R², R³, R⁴, R⁵, R⁶, R⁹, R¹⁰, R¹¹, and R¹² is, independently, H, (CH₂)ₙOR¹³, (CH₂)ₙC(O)OR¹³, (CH₂)ₙOC(O)R¹⁶, (CH₂)ₙS(O)ₘR¹³, (CH₂)ₙS(O)ₘNR¹⁴R^{15′}; (CH₂)ₙS—SR¹³; (CH₂)ₙNR¹⁴R¹⁵, (CH₂)ₙC(O)NR¹⁴R¹⁵, (CH₂)ₙOC(O)NR¹⁴R¹⁵ (CH₂)ₙNR¹⁴C(O)NR¹⁴R¹⁵, (CH₂)ₙNR¹⁴C(O)OR¹³, (CH₂)ₙNR¹⁴C(O)R¹⁶, (CH₂)ₙ O—N═CR¹⁶, (CH₂)N—N═CR¹⁶, a single D or L amino acid, a D or L di, tri, tetra or penta peptide, a combination of a D and L di, tri, tetra and penta peptide; or an oligopeptide; a PEG moiety; (CH₂)ₙNR¹⁴SO₂R¹⁶; (CH₂)ₙCH═N—OR¹⁶; (CH₂)ₙCH═N—NR¹⁴R¹⁶; C₁-C₃₀ alkyl; C₂-C₃₀; alkenyl; C₂-C₃₀ alkynyl; heterocycle or heteroaryl (e.g. triazole);
each R⁷ and R⁸, for each occurrence, is independently H, C₁-C₃₀ alkyl, C₂-C₃₀ alkenyl, C₂-C₃₀ alkynyl, C(O)OR¹³, C(O)R¹⁶, R^{d}, SO₂R¹⁶, or a nitrogen protecting group such as BOC, Fmoc or benzyl;
R¹³, for each occurrence, is independently H, alkyl, alkenyl, alkynyl, or R^{d}, each of which is optionally substituted with 1-3 nitrogen containing moieties selected from the group consisting of NR¹⁸R¹⁹ or a nitrogen containing heterocycle or heteroaryl;
each R¹⁴ and R¹⁵, for each occurrence, is independently H, alkyl alkenyl, or alkynyl, or R^{d}, each of which is optionally substituted with 1-3 nitrogen containing moieties selected from the group consisting of NR¹⁸R¹⁹ or a nitrogen containing heterocycle or heteroaryl;
R¹⁶, for each occurrence, is alkyl alkenyl, alkynyl, R^{d}, or —C₁₋₁₀alkylNR¹⁴C(O)R^{d}, each of which is optionally substituted with 1-3 nitrogen containing moieties selected from the group consisting of NR¹⁸R¹⁹ or a nitrogen containing heterocycle or heteroaryl;
R^{d} is a cholesterol moiety, optionally substituted with C(O)OR^{L}, C(O)NR^{L}R^{L′}, R^{L}, S(O)ₘR^{L}, or S(O)ₘNR^{L}R^{L′};
each R^{L} and R^{L′} is independently H, alkyl alkenyl, alkynyl or R^{d}, each of which is optionally substituted with 1-3 nitrogen containing moieties selected from the group consisting of NR¹⁸R¹⁹ or a nitrogen containing heterocycle or heteroaryl;
each R¹⁸ and R¹⁹, for each occurrence, is independently, H, alkyl alkenyl, alkynyl, or a nitrogen protecting group (e.g. BOC, Fmoc or benzyl);
m is 0, 1, or 2
each n is independently 0 to 20; and
wherein formula (I) contains at least one lipophilic group and at least one cationic group.

2. A compound of claim 1, wherein Z is absent.

3. A compound of claim 2, wherein R¹, R², R⁴ and R⁶ are H.

4. A compound of claim 3, wherein R³ is NHC(O)R¹⁶ and R⁵ is C(O)NR¹⁴R¹⁵.

5. The compound of claim 4, wherein the compound is present in a diastereomeric mixture.

6. The compound of claim 4, wherein the compound has at least a 60% diastereomeric excess of the 2R,4R configuration.

7. The compound of claim 4, wherein the compound has at least a 60% diastereomeric excess of the 2S,4R configuration.

8. The compound of claim 4, wherein the compound has at least a 60% diastereomeric excess of the 2S,4S configuration.

9. The compound of claim 4, wherein the compound has at least a 60% diastereomeric excess of the 2R,4S configuration.

10. The compound of claim 1, wherein R⁷ is H.

11. The compound of claim 1, wherein R⁷ is a nitrogen protecting group.

12. The compound of claim 1, wherein R⁷ is C(O)R¹⁶.

13. The compound of claim 12, wherein R¹⁶ is alkyl substituted with 1-3 NR¹⁸R¹⁹.

14. The compound of claim 12, wherein R¹⁶ is substituted with a nitrogen containing heterocyclyl.

15. The compound of claim 14, wherein R¹⁶ is further substituted by NR¹⁸R¹⁹.

16. The compound of claim 14, wherein the heterocyclyl is an imidazolyl.

17. The compound of claim 13, wherein R¹⁶ is

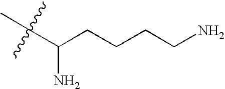

18. The compound of claim 12, wherein R¹⁶ is alkyl substituted with NH₂ and imidazolyl.

19. The compound of claim 18, wherein R¹⁶ is

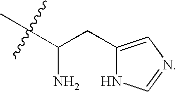

20. The compound of claim 1, wherein R¹⁶ is alkyl.

21. The compound of claim 1, wherein R¹⁶ is alkenyl.

22. The compound of claim 1, wherein R¹⁶ is alkynyl.

23. The compound of claim 1, wherein R¹⁶ is R^{d} or C₁-C₁₀ alkyl substituted with NHC(O)R^{d}.

24. The compound of claim 23, wherein R¹⁶ is R^{d}.

25. The compound of claim 24, wherein R^{d} is an unsubstituted cholesterol moiety.
